(19) United States
(12) Patent Application Publication
(10) Pub. No.: US 2020/0148937 A1
(43) Pub. Date: May 14, 2020

(54) INHIBITING HYDROCARBON HYDRATE AGGLOMERATION

(71) Applicant: COMMONWEALTH SCIENTIFIC AND INDUSTRIAL RESEARCH ORGANISATION, Australian Capital Territory (AU)

(72) Inventors: Colin WOOD, Kensington Western Australia (AU); Susan HOLMES, Kensington Western Australia (AU)

(73) Assignee: COMMONWEALTH SCIENTIFIC AND INDUSTRIAL RESEARCH ORGANISATION, Australian Capital Territory (AU)

(21) Appl. No.: 16/617,143

(22) PCT Filed: May 21, 2018
(86) PCT No.: PCT/AU2018/050482

§ 371 Date: Nov. 26, 2019

(30) Foreign Application Priority Data
May 31, 2017 (AU) 2017902061
Apr. 6, 2018 (AU) 2018901146

(51) Int. Cl. C09K 8/52; C07C 317/40; C07C 233/32; C07C 233/54; C07D 317/60

(52) U.S. Cl. CPC C09K 8/52; C07C 317/40; C09K 2208/22; C07C 233/54; C07D 317/60; C07C 233/32

(57) ABSTRACT

A process for inhibiting the formation of gas hydrates in a hydrocarbon fluid including adding to the hydrocarbon fluid, a gas hydrate anti-agglomerate which is a biodegradable anti-agglomerant derived from a naturally occurring substance.

### FIELD

The invention relates to a process and anti-agglomerant for inhibiting the agglomeration of hydrocarbon hydrates and in particular for inhibiting the agglomeration of hydrocarbon hydrates and blockages in wells, pipelines and conduits used in the oil and gas industry.

### BACKGROUND

Hydrocarbon hydrates are non-stoichiometric crystalline compounds, consisting of host water and guest hydrocarbon molecules. Hydrocarbon hydrates form at lower temperatures and high pressure and cause considerable problems in the oil and gas industry. The formation of hydrocarbon hydrates may result in a disruption in hydrocarbon transport through blockages in infrastructure such as oil and gas production wells, pipelines, and conduits used to transport hydrocarbons. The blockages occur due to the growth and agglomeration of hydrocarbon hydrate particles which ultimately turn into a solid hydrocarbon hydrate plug which can result in flow reduction, equipment damage, loss of production, shut down of mining facilities and an increased risk of explosion or release of hydrocarbons to the environment.

In order to reduce the risk of hydrocarbon hydrate plug formation, chemical inhibitors are added to the hydrocarbon flow particularly under conditions of low temperature and/or high pressure which are conducive to hydrocarbon hydrate formation. In particular, vast quantities of thermodynamic inhibitors, such as methanol, ethanol, propanol and monoethylene glycol (MEG) are provided to flow lines to shift the hydrocarbon hydrate equilibrium curve such that higher pressures and lower temperatures are required for hydrocarbon hydrate formation. Thus, operating conditions can be used which are outside of the hydrate formation conditions. Thermodynamic inhibitors are typically recycled, but the recovery process is complicated and exerts a number of technological challenges.

Alternative materials exist for hydrocarbon hydrate formation prevention and include kinetic hydrate inhibitors (KHIs) and anti-agglomerants (AAs). KHIs are water-soluble polymers that delay the nucleation of hydrate crystals. Anti-agglomerants (AAs) are surfactants, which suspend the water phase as small droplets, which ensures that the droplets are converted to small hydrate particles when the temperature decreases below the hydrate formation equilibrium condition. The hydrate particles formed are well dispersed in the liquid phase thus preventing particle agglomeration and subsequent hydrate blockage in flow lines. Alternatively, anti-agglomerants can be hydrate surface specific species, which assemble at the hydrate interface as the hydrate forms.

More recently there have been proposals to use more effective polymeric hydrate inhibitors such as those described by Kelland in the review ‘History of the Development of Low Dosage Hydrate Inhibitors’, Energy & Fuels, Vol 20, No 3 (2006) and U.S. Pat. No. 5,880,319, US2014/0256599, WO 2008/023989, U.S. Pat. No. 8,211,469 and US 2013/0098623. While polymeric hydrate inhibitors are generally active at much lower concentrations than conventional thermodynamic inhibitors they are generally more expensive.

It is an object of the present invention to overcome or ameliorate at least one of the disadvantages of the prior art, or to provide a useful alternative.

An object of a preferred embodiment of the invention is to improve the activity of hydrate anti-agglomerants. Another object of a preferred embodiment is to provide a more cost effective, more economical hydrate anti-agglomerants. Another object of a preferred embodiment is to provide an anti-agglomerant that could be used to complement the current thermodynamic inhibitors to reduce the amount of the conventional thermodynamic inhibitors and limit recycling costs. Another object of a preferred embodiment is to provide an anti-agglomerant that has minimal negative environmental impact and preferably be at least partially biodegradable, more preferably fully biodegradable most preferably with low bioaccumulation potential and low ecotoxicity.

Another object of a preferred embodiment of the invention is to provide an anti-agglomerant having corrosion inhibition properties.

Any discussion of the prior art throughout the specification should in no way be considered as an admission that such prior art is widely known or forms part of common general knowledge in the field.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### STATEMENTS OF THE INVENTION

In one aspect of the invention, there is provided a process for inhibiting gas hydrate agglomeration in a hydrocarbon phase, comprising the steps of:

(i) providing at least one oil soluble anti-agglomerant derived from a lipophilic, oil soluble naturally occurring product, a precursor to, a metabolite or a derivative thereof, and comprising at least one non-ionic hydrophilic head group;

(ii) generating repulsive forces between the gas hydrate in the hydrocarbon phase by contacting the gas hydrate with the at least one anti-agglomerant to form a plurality of dispersed gas hydrate-anti-agglomerant associated particles.

In one aspect of the invention, there is provided a process for inhibiting gas hydrate agglomeration in a hydrocarbon phase, comprising the steps of:

(i) providing at least one oil soluble anti-agglomerant derived from a lipophilic, oil soluble naturally occurring product, a precursor to, a metabolite or a derivative thereof, and comprising at least one non-ionic hydrophilic head group;

(ii) generating repulsive forces between the gas hydrates in the hydrocarbon phase by contacting gas hydrate with the at least one anti-agglomerant to form a plurality of dispersed gas hydrate-anti-agglomerant associated particles.

In a related embodiment, there is provided a process for inhibiting gas hydrate agglomeration in a slurry comprising a water-in-hydrocarbon dispersion having a plurality of water droplets dispersed in a continuous hydrocarbon phase, said method comprising the steps of:

(i) providing at least one oil soluble anti-agglomerant derived from a lipophilic, oil soluble naturally occurring product, a precursor to, a metabolite or a derivative thereof and comprising at least one non-ionic hydrophilic head group to the water-in-hydrocarbon dispersion in a transport line:

(ii) allowing anti-agglomerant to associate with gas hydrates formed at an interface of the water-hydrocarbon phase to form the slurry comprising a dispersion of gas hydrate-anti-agglomerant associated particles in the hydrocarbon continuous phase,

whereby repulsive forces between the gas hydrate-anti-agglomerant associated particles and/or attractive forces between the gas hydrate-anti-agglomerant associated particles and the hydrocarbon continuous phase of the slurry maintain the gas hydrate-anti-agglomerant associated particles in substantially dispersed form.

In another aspect, the invention provides for a process for inhibiting gas hydrate crystal agglomeration in a hydrocarbon phase, wherein under gas hydrate crystal formation conditions, one or more of the hydrocarbons are trapped within structures formed from water molecules present with the one or more hydrocarbons, said method comprising the steps of:

(i) providing at least one oil soluble anti-agglomerant derived from a lipophilic, oil soluble naturally occurring product, a precursor to, a metabolite or a derivative thereof, and comprising at least one non-ionic hydrophilic head group;

(ii) contacting the gas hydrate crystals during or after their formation with the at least one anti-agglomerant whereby the hydrophilic head groups of the at least one anti-agglomerant molecules associate with the water molecules of the gas hydrate crystals to form a plurality of associated gas crystal-anti-agglomerant particles whereby repulsive forces between the lipophilic tail of the associated gas crystal-anti-agglomerant particle formed prevent agglomeration of the gas hydrate crystals into gas hydrate clusters.

In a further aspect, there is provided a process of inhibiting the formation of gas hydrate blockages in a hydrocarbon well, pipeline and/or conduit carrying a hydrocarbon phase, the process comprising the steps of:

preventing a gas hydrate of blockage size forming in the hydrocarbon phase by maintaining a dispersion of gas hydrate crystals formed in the hydrocarbon phase by contacting the gas hydrate crystals with at least one oil soluble anti-agglomerant derived from a lipophilic, oil soluble naturally occurring product, a precursor to, a metabolite or a derivative thereof, and comprising at least one non-ionic hydrophilic head group thereby forming a plurality of associated gas crystal-anti-agglomerant particles,

whereby repulsive forces between the associated gas crystal-anti-agglomerant particles prevent agglomeration of the gas hydrate crystals into a solid blockage size plug.

In another aspect, the invention provides a use of an oil soluble anti-agglomerant to prevent gas hydrate agglomeration in a hydrocarbon phase, said at least one oil soluble anti-agglomerant derived from a lipophilic, oil soluble naturally occurring product, a precursor to, a metabolite or a derivative thereof and comprising at least one non-ionic hydrophilic head group.

In another aspect, there is provided a use of an anti-agglomerant of the invention in the prevention of gas hydrate cluster formation, said anti-agglomerant derived from a lipophilic, oil soluble naturally occurring product, a precursor to, a metabolite or a derivative thereof and comprising at least one non-ionic hydrophilic head group.

In a further aspect, the invention provides a process for inhibiting the agglomeration of hydrates in a hydrocarbon fluid comprising adding to the fluid an effective amount of an anti-agglomerant derived from a lipophilic, oil soluble naturally occurring product, a precursor to, a metabolite or a derivative thereof and comprising at least one non-ionic hydrophilic head group.

In another aspect, the invention provides for use of the anti-agglomerant as described to inhibit the corrosion of a material such as ferrous metal, for example, steel such as stainless steel, the various alloy steels and carbon steel. In such an embodiment, when the anti-agglomerant is added to a fluid, it decreases the rate of corrosion of a ferrous metal material in contact with the fluid.

Accordingly in another aspect, there is provided a process as described herein wherein the hydrocarbon phase is in contact with a metal, particularly ferrous metal, and the anti-agglomerant of the invention is provided in an amount sufficient to inhibit corrosion of the metal.

It will be understood that the gas hydrates form under suitable conditions that promote encapsulation or trapping of the one or more of the hydrocarbons within cage-like structures formed from water molecules present with the one or more hydrocarbons. Such conditions are well known to the persons skilled in the art and require that water is present in the hydrocarbon phase. The water is entrained in the hydrocarbon phase in the form of water droplets which are dispersed in the continuous oil phase as a water-in-hydrocarbon emulsion. During gas hydrate formation which occurs under suitable conditions of pressure and temperature, gas hydrate crystals or shells form at the interface between the water droplets and the surrounding hydrocarbon oil phase thereby making a solid shell around the water droplets. This process leads to formation of a dispersion that changes progressively from a water-in-oil emulsion to a hydrate-in-oil suspension or slurry. The hydrate encrusted water droplets then agglomerate, increasing into larger hydrate masses or agglomerates over time until all of the water droplets are consumed. The agglomeration of the hydrate particles leads to an increase in slurry viscosity, which over time, results in a plug.

As the surfaces of the gas hydrate-anti-agglomerant associated particles formed during the process of the invention are lipophilic, the gas hydrate-anti-agglomerant associated particles are attracted to the hydrocarbon medium and repel additional gas hydrate-anti-agglomerant associated particles hydrate shells dispersed in the continuous phase thereby preventing agglomeration into larger hydrate masses. These characteristics result in maintenance of the gas hydrate-anti-agglomerant associated particles in a dispersed form which maintains the water present as very small droplets such that gas hydrates formed there from are small in nature and also remain dispersed in the oil phase. In other words, a stable dispersion is formed.

The slurry of resultant gas hydrate-anti-agglomerant associated particles hydrate-in-hydrocarbon dispersion/suspension has a relative viscosity lower than that of slurry comprising equivalent particles formed under the equivalent conditions in absence of the oil soluble anti-agglomerant described herein. Equivalent conditions include temperature, pressure, section of line, flow rate, hydrocarbon phase composition, water cut, etc.

It will be appreciated that the repulsive forces generated between the gas hydrate-anti-agglomerant associated particles prevents agglomeration of the particles into larger gas hydrate clusters of a size that forms a solid hydrate well, pipeline or conduit blockage. Furthermore, it will be understood that a hydrate cluster is a mass of agglomerated hydrate which is of sufficient dimensions to substantially plug, block or impede flow through a well, pipeline and/or conduit through which the hydrocarbon phase flows. It will be understood that gas hydrate clusters within the meaning of the present invention are those of size that form a solid hydrate well, pipeline or conduit blockage. In such cases, it is usual that the solid hydrate cluster is of a size that has at least one dimension that is greater than the diameter of the line. Thus, it will be understood that repulsive forces between the gas hydrate-anti-agglomerant associated particles prevents agglomeration of the particles into larger gas hydrate clusters of a size that forms a solid hydrate well, pipeline or conduit blockage.

Suitably, after the contacting step, the gas hydrate-anti-agglomerant associated particles remain dispersed in the hydrocarbon phase. This is a particularly desirable benefit of the process of the present invention, as the repulsive forces that maintain well dispersed gas hydrate-anti-agglomerant associated particles mean the particles cannot agglomerate in any significant way to form clusters of hydrates of sufficient size to block conduits and/or to impede hydrocarbon transport or flow through the conduit.

It is understood that gas hydrates form directly in the hydrocarbon phase or form deposit on walls of a line through which the hydrocarbon phase is being transported.

It will be appreciated that when the appropriately selected lipophilic, oil soluble naturally occurring product, precursor to, metabolite or derivative thereof, and the appropriately selected at least one non-ionic hydrophilic tail group are combined to form the anti-agglomerant, the degree of lipophilicity and hydrophilicity of the respective lipophilic and hydrophilicity must be sufficiently balanced so as to provide an oil soluble anti-agglomerant that is capable of associating with water droplets and/or hydrates and that the resultant repulsive forces generated between the gas hydrate-anti-agglomerant associated particles are sufficient to maintain the gas hydrate-anti-agglomerant associated particles in substantially dispersed form in the hydrocarbon phase. In other words, the anti-agglomerant forms a stable water-in-hydrocarbon dispersion. It will be appreciated that where gas hydrates formed in the hydrocarbon phase are contacted with the at least one anti-agglomerant to form a plurality of gas hydrate-anti-agglomerant associated particles the contacting steps results in an association that generate repulsive forces between the associated particles. Sufficiently strong repulsive forces between the gas hydrate-anti-agglomerant associated particles are necessary to maintain the gas hydrate-anti-agglomerant associated particles in substantially dispersed form, for example, in a stable dispersion. Thus suitably, the dispersion formed is a stable dispersion. A stable dispersion is one that is kinetically stable over a desired period. By kinetically stable, it is meant that the dispersed particles do not: float (cream) to the surface of, or sediment to the bottom of, the continuous phase, flocculate, coalesce, coagulate or agglomerate to form particles of a greater size than the initial average particle size over a desired time period of stability. Suitably, the time period ranges from 30 mins to 20 days, more preferably from 1 hour to 10 days, more preferably from 8 hours to 8 days, more preferably from 1 day to 10 days, most preferably from 1 to 2 days. The stability of the dispersion can be assessed by methods known to the skilled person, for example, monitoring torque in the system, for example, using a high-pressure autoclave, or by considering rocking cell data, or by micromechanical force measurement, light scattering experiments or by considering the zeta potential of the dispersion. It will be appreciated that in a stable dispersion, the average particle size does not deviate by more than 5-50% over the desired period of stability.

Suitably, the hydrocarbon phase and/or slurry comprising hydrate and hydrocarbon is transported through a line, for example, through a well, a flow line, a pipe or other type of conduit.

It is appreciated that gas hydrate formation is rapid and typically occurs after the system has been shut-in. In typical cases, where a blockage develops, the pressure difference across the plug/blockage becomes substantial even to the degree that the plug is caused to propel along the pipeline.

Suitably, the anti-agglomerant used herein is a surface active substance.

In one embodiment, the hydrocarbon phase includes a gas dominant hydrocarbon mixture which can further comprise water together with a liquid hydrocarbon phase. In another embodiment, the hydrocarbon phase includes a liquid dominant hydrocarbon mixture which can further comprise water and gas hydrocarbon phase. Suitably, the hydrocarbon phase is a water-in-hydrocarbon dispersion, whereby water droplets are the discontinuous phase dispersed in the hydrocarbon continuous phase. Suitably, the hydrocarbon phase comprises one or more hydrocarbons. It will be understood that the hydrocarbon phase may include hydrocarbons that are in a fluid form, for example, a gas or a liquid form, or mixtures thereof. Furthermore, it will be understood that substantially all of the hydrocarbon phase may be in a gas or a liquid phase, or mixtures thereof, depending on the local environmental conditions of pressure and temperature in particular. For example, a hydrocarbon that is a gas at ambient temperatures and/or ambient pressures may be in a liquid phase at suitably low temperatures and/or high pressures.

Maintenance of a stable dispersion ensures that the viscosity of the hydrocarbon phase and/slurry of particles does not block or impede pumping and/or transport through the line.

The anti-agglomerant can be added before, during or after hydrate formation.

It will be appreciated that water must be present in the hydrocarbon phase for hydrate formation. The amount of water present is determined by the water cut which is the ratio of water volume to the volume of total fluid present, for example, as it flows through the line. However, with water cuts of >50%, more particularly >60%, more particularly >70%, more particularly >80%, more particularly >90% watercut, there may be a risk of the formation of a hydrocarbon-in-water emulsion in which case the anti-agglomerant described may be less effective or may require addition of a surfactant to prevent hydrocarbon-in-water emulsion formation and to ensure a stable dispersion can be maintained. The skilled person can readily determine the water cut in a hydrocarbon transport line using a water cut meter. Accordingly, in a preferred embodiment of the invention, the hydrocarbon phase has an associated water cut off from >0 to 50%, more preferably from >0 to 45%, more preferably from >0 to 40%, more preferably from >0 to 35%, more preferably from >0 to 30%, more preferably from >0 to 25%, more preferably from >0 to 20%, more preferably from >0 to 15%, more preferably from >0 to 10%, more preferably from >0 to 5%. Most preferred water cuts are of about 0.5%, 1%, 1.5%, 2%, 2.5%, 3%, 3.5%, 4%, 4.5%, or 5% water-in-hydrocarbon phase.

Hydrate agglomeration and the risk of plugging or blockage can be monitored by the observation of one or more of: a pressure drop along a flow line, an increase in the hydrate volume fraction in the line, an increase in the hydrate/hydrocarbon slurry relative viscosity, and a decrease in flow rate. Suitably, the decrease in flow rate is a decrease of ≥5%, ≥10%, ≥15%, ≥20%, ≥25%, ≥30%, ≥35%, ≥40%, ≥45%, ≥50%, ≥55%, ≥80%, ≥85%, ≥70%, ≥75%, ≥80%, ≥85%, ≥90%, a 95%, or 100% of a starting flow rate. The skilled person will appreciate that the decrease in flow rate indicative of a blockage or increased viscosity associated with significant hydrate agglomeration will depend on the pressure in the reservoir and hydrocarbon phase at a given timepoint.

Preferably, using the described anti-agglomerant in the process of the invention, the ΔP_{flowline} is maintained at a value <300 psi, whereby ΔP_{flowline} is the pressure drop along the line (i.e., well, flow line, pipe or conduit) through which the hydrocarbon phase is transported. Preferably, the Φ_{hyd} is maintained at a value <0.10, whereby ϕ_{hyd} is the hydrate volume fraction in the line. Further preferably, the μᵣ is maintained at a value <10, whereby μᵣ is the hydrate slurry relative viscosity. Maintenance of one or more of these conditions may result in an acceptably low risk of hydrate plugging in the line in question. Above these conditions there may be an increased likelihood of hydrate agglomeration to the degree that causes a plug or blockage in an associated line.

As discussed above, maintaining the gas hydrate-anti-agglomerant associated particles in a substantially stable dispersed form prevents the particles from agglomerating and/or coalescing, or otherwise becoming associated with each other to form larger clusters of hydrates of line blocking size. For example, the average particle size/volume and/or particle diameter of the dispersed gas hydrate-anti-agglomerant associated in the hydrocarbon phase/slurry resulting from the present process are smaller than equivalent particles formed under the same conditions in absence of the oil soluble anti-agglomerant described herein. Preferably, the gas hydrate-anti-agglomerant associated particles formed are several orders of magnitude smaller than gas hydrates formed under the same conditions in the absence of anti-agglomerant associated particles. The particle sizes can be determined for by light scattering experiments.

It will be appreciated that the at least one non-ionic hydrophilic head group of the anti-agglomerant is attracted to and associates with water droplets and/or hydrate molecules on the surface/outside of a hydrate shell, in a proximal position to the hydrate surface, while the lipophilic tails remains in a distal position to the hydrate surface and thus are free to repel the distally positioned lipophilic tails of neighbouring gas hydrate-anti-agglomerant associated particles. Thus, the anti-agglomerant molecule of the invention serves to disrupt the normal agglomeration tendency of the gas hydrates thereby preventing the formation of progressively bigger solid dusters of hydrate particles.

Suitably, the anti-agglomerant is inert, that is, it does not react with water, a hydrocarbon or other components of the hydrocarbon phase or otherwise to break down under hydrate forming conditions.

Advantageously, such use is preferably in natural gas recovery, particularly where the gas is transported in cold conditions, both onshore and offshore.

Indeed, preferably, the use is as an alternative or supplement to thermodynamic inhibitors (e.g. MEG) in applications where thermodynamic inhibitors are used.

It will be further understood that an effective amount of anti-agglomerant to ensure a dispersion of particles is formed and maintained should be used. Suitably, the anti-agglomerant may be added to the hydrocarbon phase and/or slurry in an amount in the range of from 0.01 to 5% by weight based on the weight of water in the hydrocarbon phase and/or slurry.

In a preferred embodiment, the anti-agglomerant is added to the hydrocarbon phase and/or slurry with one or more thermodynamic hydrate inhibitors selected from the group consisting of methanol, ethanol, isopropanol, monoethyleneglycol and mixtures thereof. Suitably, the weight ratio of the anti-agglomerant to thermodynamic hydrate inhibitor is in the range of from 0.001 to 2%.

In a particularly preferred embodiment, the process described above additionally provides corrosion inhibition wherein the hydrocarbon phase and/or slurry is in contact with a metal, particularly ferrous metal, and the anti-agglomerant is present in an amount sufficient to inhibit corrosion of the metal.

Preferably, the process described herein is useful in oil and/or natural gas recovery, particularly where the hydrocarbon phase is transported in cold conditions, both onshore and offshore.

Suitably, the anti-agglomerant as described herein may be used as an alternative or supplement to thermodynamic inhibitors (MEG) in applications where thermodynamic inhibitors are used.

Suitably, the hydrocarbon phase comprises one or more hydrocarbons, preferably selected from C₁-C₁₀ hydrocarbons, more preferably, C₁-C₅ hydrocarbons, more preferably still, natural gas. Natural gas is composed primarily of methane, but may also contain ethane, propane and/or other hydrocarbons.

Suitably, the contacting/associating step occurs during or after gas hydrate formation. The anti-agglomerant may be added to the hydrocarbon phase before it enters the conduit or while it is flowing through the conduit.

Preferably, the anti-agglomerant is environmentally benign, biodegradable, and/or non-toxic to plants, animals, marine life and/or insects. Suitably, the at least one naturally occurring product is a biologically active compound or precursor thereto or metabolite thereof, or is found in a living organism, such as a microorganism, an insect, a plant, and/or animal. Several advantages arise from use of the anti-agglomerant described herein as they are environmentally benign materials that are biodegradable. Disposal of these anti-agglomerants into the environment is less of an issue because they are naturally derived.

Advantageously, as the anti-agglomerant is oil soluble, this results in reduced partitioning of the anti-agglomerant into the aqueous phase. This is favourable for environmental reasons as water treatment is more convenient as the anti-agglomerant remains in the hydrocarbon phase.

Furthermore still, the preferred anti-agglomerant molecules of the invention do not result in oil in water emulsion formation.

Preferably, the anti-agglomerant does not form a water-in-hydrocarbon emulsion, during the process of the invention. Water-in-oil emulsions formed under the turbulent conditions found in hydrocarbon wells, pipelines and/or conduits are frequently difficult to treat and/or phase disperse/separate.

Furthermore, as demonstrated herein, for the anti-agglomerants tested, the performance for preventing hydrate agglomeration is at least comparable to existing more toxic anti-agglomerants.

In a preferred embodiment, the anti-agglomerant used in the process of the invention has a hydrophilic-lipophilic balance (HLB) of between 3 and 6. By the “hydrophilic-lipophilic balance” (HLB) of an anti-agglomerant molecule, it is mean the degree to which the anti-agglomerant is hydrophilic or lipophilic. The HLB is typically determined by calculating values for the different regions of the molecular. HLB values are typically calculating using Davies' approach (see Gas/Liquid and Liquid/Liquid Interfaces, Proceedings of 2nd International Congress Surface Activity, Butterworths, London 1957, 426, A QUANTITATIVE KINETIC THEORY OF EMULSION TYPE. I. PHYSICAL CHEMISTRY OF THE EMULSIFYING AGENT, J. T. DAVIES, the content of which is hereby incorporated by reference) which considers the weighted contribution of hydrophilic or lipophilic units toward overall HLB. For example, a HLB value of 0 corresponds to a completely lipophilic/lipophilic molecule, while a HLB value of 20 corresponds to a completely hydrophilic/lipophobic molecule. Typically, a compound having a HLB of from about 3 to 8 would be expected to form a water-in-oil (W/O) emulsion. Using the anti-agglomerant having a HLB balance of between 3 and 6, more preferably 4 and 5, most preferably 4 or 5, reduces the likelihood of the anti-agglomerant forming an oil in water emulsion under the conditions encountered during hydrocarbon processing/transport.

Preferred anti-agglomerants are oil soluble compounds which comprise at least one lipophilic tail component and at least one non-ionic hydrophilic head group component as described herein.

Suitably, the at least one lipophilic tail component is derived from an oil soluble naturally occurring product, a precursor to, a metabolite or a derivative thereof, as described herein.

Preferably, the at least one non-ionic hydrophilic head group component of the oil soluble anti-agglomerant is derived from a naturally occurring product, a precursor to, a metabolite or a derivative thereof, as described herein.

Preferably, the at least one non-ionic hydrophilic head group component of the anti-agglomerant is derived from a precursor compound which is a water soluble naturally occurring product, a precursor to, a metabolite or a derivative thereof, as described herein.

Most preferably, both of the at least one lipophilic tail component and the at least one non-ionic hydrophilic head group component are derived from a naturally occurring product, a precursor to, a metabolite or a derivative thereof, as described herein.

An oil/fat soluble compound is one that is soluble in a hydrocarbon phase to at least 0.1 to 5 wt %, preferably at least 0.25 to 3%, more preferably 0.5 to 2%, and most preferably from 0.85 to 1 wt % based on the amount of the water phase present.

By “derived from a naturally occurring product, a precursor to, a metabolite or a derivative thereof”, it is meant that the lipophilic tail component and/or the at least one non-ionic hydrophilic head group component is chemically no more than 1 or 2 chemical transformation steps away from the naturally occurring product.

A “naturally occurring product” as described herein is a naturally found substance, and includes a biological precursor to, or a biological metabolite of, or a synthetically derived version of such a naturally found substance, wherein the substance pertains to the biochemistry and/or physiology of living organisms, including plants, fish, animals including marine animals and humans, insects and/or microorganisms. Such substances will have a pharmacological or biological presence, activity and/or role. The naturally occurring product as described herein may be an organic, inorganic or an organometallic substance or molecule, which may be extracted from any naturally or non-naturally occurring source; prepared by a genetic engineering method; or may be derived from a total or partial chemical synthesis of the substance. The naturally occurring product may be found in nature as a primary or a secondary metabolite. Preferably the naturally occurring product is readily isolated from natural or non-natural sources. Naturally occurring products which may be isolated by extraction, for example, solvent extraction, from suitable sources are preferred. This is particularly true in the cases of substances that can be readily extracted in large quantities using economical viable conditions. The naturally occurring product may be derived from a fermentation process or by chemical synthesis, via total or semi synthetic methods. It will be understood that where such a product can be prepared synthetically, the naturally occurring product as intended herein includes synthetic analogues of the naturally occurring substance. The naturally occurring product may be produced in nature via biosynthetic pathways which may include: (i) photosynthesis involving the gluconeogenesis pathway which converts monosaccharides to polysaccharides, (ii) the acetate pathway which results in fatty acids and polyketides, (iii) the Shikimate pathway which produces aromatic amino acids and phenylpropanoids including phenylalanine, tyrosine and their derivatives, (iv) mevalonate and methyletrythritol phosphate pathways which produce terpenoids and steroids and (v) amino acid pathways that result in alkaloids.

Exemplary naturally occurring products belong to the following classes of naturally occurring substances: nucleosides, nucleotides, nucleic acids, amino acids, proteins, hormones, carbohydrates, cofactors, lipids, fats, proteins, enzymes, pigments, alkaloids, phenylpropanoids, polyketides, terpenoids, vitamins, provitamins, tetrapyrroles.

It will be understood that different naturally occurring products have different overall lipophilicities and hydrophilicities which are determined by the number and type of chemical functional groups present in the product, for example, polar or non-polar groups or functionalities. The skilled person can readily determine the lipophilicities and hydrophilicities of any given naturally occurring product as described herein using conventional methods in the art, for example, solubility or partitioning studies.

Suitably, the one or more non-ionic hydrophilic head group components and the one or more lipophilic tail components of the anti-agglomerants correspond to separate or discreet parts or portions of a molecule whereby each of the components are found within the structure of the molecule. For example, the molecule may take the following form:
non-ionic hydrophilic head group-lipophilic tail

Alternatively, the non-ionic hydrophilic head group component may be coupled to the lipophilic tail group component via a linker functionality, L:
non-ionic hydrophilic head group-L-lipophilic tail

The linker L may be derived from suitable couplable functional groups which may be found on the respective head group and tail components or may be delivered from a further compound required to enable the coupling reaction. For example, when group R* directly reacts with group R**, a couple/linker L is formed between the pair of molecule fragments shown below in (a) or when a coupling reagent is use, the reagent may form part of the linker functionality as shown in (b):

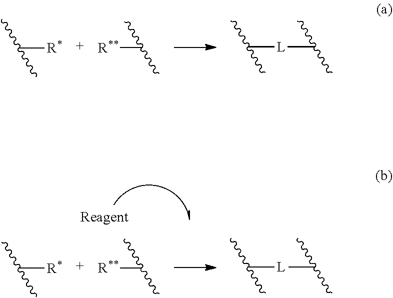

Suitable functional groups capable of forming a couple must be available on the fragments to be coupled. In certain cases, one or more of the functional groups may be activated to initiate/enable coupling. Such activation methods are known in the art. For example, where a carboxylic acid group is involved in the coupling reaction, an activated carboxylic acid functional group readily couples with other functional groups to form a linkage, for example, activated carboxylate reacts with amine to form an amide linkage. Such coupling reactions typically involve use of coupling agent reagents such as carbodiimides, diimidazoles, or succinimides. Examples of suitable coupling agents for carboxylic acid based coupling include carbonyl diimidazole, dicyclohexylcarbodiimide, N-hydroxylsuccinimide. These agents may or may not be incorporated into the final molecule after the coupling reaction as discussed above.

As explained above, preferred non-ionic head hydrophilic groups may be derived from precursor compounds comprising functional groups suitable for coupling to fatty chains, for example, non-ionic carboxylic acid based head groups, oxygen based head groups, nitrogen head groups, sulfur head groups and methylbenzoate head groups. Suitable functional groups include:
carbonyl based head groups comprising for example an aldehyde, ketone, ester, amide, imide, carboxylic acid, or carboxylic acid ester functionality;
oxygen based head groups comprising an alcohol or ether functionality;
nitrogen based head groups comprising an amine, imine or guanidine functionality; and
sulfur head groups comprising a sulfhydryl, sulfonamide, thionoester, thioloester, thiozole, and thiophene functionality.

In the example, above, suitable functional groups include a carboxylate, an amine, an alcohol, and an aldehyde group. Where an amine functional group is coupled to a carboxylic acid functional group, the couple or linkage formed is an amide linkage. Where an alcohol is coupled to a carboxylic acid, the linkage is an ester linkage. Where an aldehyde group is coupled to an amine, the linkage is an imine linkage. In one embodiment, the non-ionic hydrophilic head group can be derived from an amino acid. Suitably, the amino acid may be a straight or branched chain amino acid. Preferably, the amino acid is glycine, alanine, serine, threonine, cysteine, valine, leucine, isoleucine, methionine, aspartic acid, glutamic acid, asparagine, glutamine, lysine or arginine. Preferably, the amino acid is a ring bearing amino acids, for example, proline, phenylalanine, tyrosine, tryptophan or histidine. A preferred amino acid is phenylalanine.

As explained above, preferably the lipophilic tail component of the anti-agglomerant as described herein is derived from a lipophilic, oil soluble naturally occurring product, precursor, metabolite, or derivative thereof. Furthermore, preferred anti-agglomerants are oil soluble. Examples of oil soluble naturally occurring products include non-saponifiable lipids which include for example fat soluble fat soluble steroids, terpene carotenoids, fat soluble vitamins or coenzymes such as coenzyme Q or ubiquinones, and fat soluble derivatives, precursors, or metabolite thereof. In a preferred embodiment, the lipophilic tail component of preferred anti-agglomerants is derived from a fat soluble vitamin selected from the group consisting of: vitamin A, D, E and K, and derivatives, metabolites thereof and precursors thereto. Vitamin E is particularly preferred basis for the lipophilic tail component of the anti-agglomerant molecules used herein. In some embodiments, the lipophilic tail components of the anti-agglomerant are derived from biocompatible substances including fatty acids, fatty amines, fatty esters, fatty aldehydes, fatty ethers, fatty nitriles, or fatty alcohols. Fatty acids, fatty amines, fatty aldehydes, or fatty alcohols are most preferred as these compounds comprise readily couplable functional groups which can be used to link the lipophilic tail component and the non-ionic hydrophilic head group component as described herein to form the anti-agglomerant of the invention. Preferred lipophilic tails are derived from molecules comprising a fatty chain, for example, naturally occurring oil soluble fatty acids, fatty amines, fatty aldehydes, or fatty alcohols. Suitably, the fatty chain a C₃-C₂₄ carbon frame, which can be saturated or unsaturated. Saturated fatty chain molecules do not contain any conjugated double bonds, while unsaturated fatty chain molecules can be mono-unsaturated (one double bond) or poly-unsaturated (more than one double bond). Saturated fatty chain molecules, particularly fatty acids are typically found in animal fats, whereas unsaturated fatty acids are found in plants. Cis and trans unsaturated fatty chain molecules are suitable, however, cis unsaturated fatty chain molecules are more commonly found in nature and are thus preferred. Cis and trans unsaturated fatty acids are also suitable, however, cis unsaturated fatty acids are more commonly found in nature and are thus preferred. Straight chain fatty acids such as Tridacanoic acid, Dodecanoic acid, Undecanoic acid, Decanoic acid, Nonanoic Acid, Octanoic Acid, Heptanoic acid. However, n-Octanoic acid, Decanoic acid and Tridecanoic acid are particularly preferred. Phytanic acid (3,7,11,15-Tetramethylhexadecanoic acid) is one example of a suitable branched fatty acid.

In other embodiments, the lipophilic tail group component of the anti-agglomerant is derived from a ketoacyl or isoprene precursor or building block. For example, the oil soluble naturally occurring substance can be a fatty acyl lipid, the synthesis of which may involve chain elongation of an acetyl-CoA with malonyl-CoA (or methylmalonyl-CoA) groups. Fatty acyls are typically exemplified by fatty acids and conjugates, eicosanoids, fatty alcohols, fatty esters and rhamnolipids.

Preferably, the biodegradable naturally occurring product forming the lipophilic tail group of the anti-agglomerant is an oil soluble substance selected from the group of naturally occurring lipids consisting of: Fatty Acyls [FA]. Glycerolipids [GL], Sphingolipids [SP]. Sterol Lipids [ST], Prenol Lipids [PR], Saccharolipids [SL], and Polyketides [PK]. Precursors to, metabolites of or derivative thereof are also included within the scope of the present invention. The designation given in square brackets [ . . . ] corresponds to the classifications of lipid provided by the LIPID MAPS® Lipidomics Gateway, a free resource sponsored by the Wellcome Trust and available at http://www.lipidmaps.org/. Specific examples of lipids can be conveniently found under the referenced classifications at this resource, the specific examples of which are incorporated herein by reference.

Other entitles having ionic head groups, for example, Glycerophospholipids [GP] must be modified such that the head group is not ionic if they are to be used as the non-ionic hydrophilic head group of the anti-agglomerant as described herein.

In a particularly preferred embodiment, the lipophilic tail group of the anti-agglomerant is an oil soluble substance selected from the group consisting of: Sterol Lipids [ST], Prenol Lipids [PR], and Polyketides [PK].

Suitably the Fatty Acyls [FA] include Fatty Acids and Conjugates [FA01], Octadecanoids [F62] Eicosanoids [FA03], Docosanoids [FA04] Fatty alcohols [FA05] Fatty aldehydes [FA06], Fatty esters [FA07], Fatty amides [FA08], Fatty nitriles [FA09], Fatty ethers [FA10] Hydrocarbons [FA11]. Oxygenated hydrocarbons [FA12], Fatty acyl glycosides [FA13], and Other Fatty Acyls [FA00]. For example, fatty esters include fatty alcohol acetate esters, branched fatty acid esters, epoxy acid esters, fatty acyl carnitines, fatty acyl CoAs, hydroxy fatty acids, methyl esters, propyl esters.

Suitably the Glycerolipids, include Monoradylglycerols [GL01], Diradylglycerols [GL02], Triradylglycerols [GL03], Glycosylmonoradylglycerols [GL04], Glycosyldiradylglycerols [GL05], and Other Glycerolipids [GL00]. For example. Glycerolipids include monoacylglycerols, diacylglycerols, triacylglycerols.

Suitably the Glycerophospholipids [GP] include Glycerophosphocholines [GP01], Glycerophosphoethanolamines [GP02], Glycerophosphoserines [GP03], Glycerophosphoglycerols [GP04], Glycerophosphoglycerophosphates [GP05], Glycerophosphoinositols [GP06], Glycerophosphoinositol monophosphates [GP07], Glycerophosphoinositol bisphosphates [GP08], Glycerophosphoinositol trisphosphates [GP09], Glycerophosphates [GP10], Glyceropyrophosphates [GP11], Glycerophosphoglycerophosphoglycerols [GP12], CDP-Glycerols [GP13], Glycosylglycerophospholipids [GP14], Glycerophosphoinositolglycans [GP15], Glycerophosphonocholines [GP16], Glycerophosphonoethanolamines [GP17], Di-glycerol tetraether phospholipids (caldarchaeols) [GP18], Glycerol-nonitol tetraether phospholipids [GP19], Oxidized glycerophospholipids [GP20], and Other Glycerophospholipids [GP00].

Suitably the Sphingolipids [SP] include Sphingoid bases [SP01], Ceramides [SP02], Phosphosphingolipids [SP03], Phosphonosphingolipids [SP04], Neutral glycosphingolipids [SP05], Acidic glycosphingolipids [SP06], Basic glycosphingolipids [SP07], Amphoteric glycosphingolipids [SP08], Arsenosphingolipids [SP09], and Other Sphingolipids [SP00].

Suitably the Sterol Lipids [ST] include Sterols [ST01], Steroids [ST02], Secosteroids [ST03], Bile acids and derivatives [ST04], Steroid conjugates [ST05], and Other Sterol lipids [ST00].

Suitably the Prenol Lipids (PR) include Isoprenoids [PR01], Quinones and hydroquinones [PR02], Polyprenols [PR03], Hopanoids [PR04], and Other Prenol lipids [PR00].

Suitably the Saccharolipids [SL] include Acylaminosugars [SL01], Acylaminosugar glycans [SL02], Acyltrehaloses [SL03], Acyltrehalose glycans [SL04], Other acyl sugars [SL05], Other Saccharolipids [SL00].

Suitably the Polyketides [PK] including Linear polyketides [PK01], Halogenated acetogenins [PK02], Annonaceae acetogenins [PK03], Macrolides and lactone polyketides [PK04], Ansamycins and related polyketides [PK05], Polyenes [PK06], Linear tetracyclines [PK07], Angucyclines [PK08], Polyether antibiotics [PK09], Aflatoxins and related substances [PK10], Cytochalasins [PK11], Flavonoids [PK12], Aromatic polyketides [PK13], Non-ribosomal peptide/polyketide hybrids [PK14], Phenolic lipids [PK15] and Other Polyketides [PK00].

### Fatty Acyls [FA]

Preferably, the Fatty Acids and Conjugates [FA01] are selected from the group consisting of: Straight chain fatty acids, Branched fatty acids, Unsaturated fatty acids, Hydroperoxy fatty acids [FA0104], Hydroxy fatty acids [FA0105], Oxo fatty acids [FA0106], Epoxy fatty acids [FA0107], Methoxy fatty acids [FA0108], Halogenated fatty acids [FA0109], Amino fatty acids [FA0110], Cyano fatty acids [FA0111], Nitro fatty acids [FA0112], Thia fatty acids [FA0113], Carbocyclic fatty acids [FA0114], Heterocyclic fatty acids [FA0115], Mycolic acids [FA0116], and Dicarboxylic acids [FA0117].

Preferably, the Octadecanoids [FA02] are selected from the group consisting of: 12-oxophytodienoic acid metabolites [FA0201], Jasmonic acids [FA0202], Phytoprostanes [FA0203], Phytofurans [FA0204], Octadeca-1,2-dioxolanes [FA0205], and Other Octadecanoids [FA0200].

Preferably, the Eicosanoids [FA03] are selected from the group consisting of: Prostaglandins [FA0301], Leukotrienes [FA0302], Thromboxanes [FA0303], Lipoxins [FA0304], Hydroxy/hydroperoxyeicosatrienoic acids [FA0305], Hydroxy/hydroperoxyeicosatetraenoic acids [FA0306], Hydroxy/hydroperoxyeicosapentaenoic acids [FA0307], Epoxyeicosatrienoic acids [FA0308], Hepoxilins [FA0309], Levuglandins [FA0310], Isoprostanes [FA0311], Isofurans [FA0313], Eicosa-1,2-dioxolanes [FA0315], Resolvin Es [FA0314], Clavulones [FA0312], and Other Eicosanoids [FA0300];

Preferably, the Docosanoids [FA04] are selected from the group consisting of: Neuroprostanes [FA0401], Neurofurans [FA0402, Docosa-1,2-dioxolanes [FA0406], Resolvin Ds [FA0403], Protectins [FA0404], Maresins FA0405], and Other Docosanoids [FA0400].

Fatty alcohols [FA05], which are long chain, usually high-molecular-weight, straight-chain primary alcohols, but can also range from as few as 4-6 carbons to as many as 22-26, derived from natural fats and oils, and are typified by fatty alcohols such as lauryl, stearyl, and oleyl alcohols.

Fatty aldehydes [FA06], which are aldehydes with a “fatty” aliphatic carbon chain attached that is typically eight carbon or more in length. Straight chain, saturated aldehydes include octanal (C₈), nonanal (C₉), decanal (C₁₀), undecanal (C₁₁), dodecanal (C₁₂), tridecanal (C₁₃), tetradecanal (C₁₄), pentadecanal (C₁₅), hexadecanal (C₁₆), octadecanal (C₁₈), icosanal (C₂₀), docosanal (C₂₂), etc. Chains may also be unsaturated, or branched. Fatty acid esters are esters that result from the combination of a fatty acid with an alcohol, for example, when the alcohol is glycerol, the fatty acid esters produced can be monoglycerides, diglycerides, or triglycerides. Preferably, the Fatty esters [FA07] are selected from the group consisting of: Wax monoesters [FA0701], Wax diesters [FA0702], Cyano esters [FA0703], Lactones [FA0704], Fatty acyl CoAs [FA0705], Fatty acyl ACPs [FA0706], Fatty acyl carnitines [FA0707], and Fatty acyl adenylates [FA0708], Fatty acid amides are amides formed from a fatty acid and an amine, for example, an ethanolamine. They typically have the formula RC(O)N(H)CH₂CH₂OH. Fatty acid amides include fatty acid primary amides which contain the functionality RC(O)NH₂, for example, oleamide. Preferably, the Fatty amides [FA08] are selected from the group consisting of: Primary amides [FA0801], N-acyl amines [FA0802], Fatty acyl homoserine lactones [FA0803], and N-acyl ethanolamines (endocannabinoids) [FA0804], Fatty nitriles are esters of hydrogen cyanide derived from fatty acids and have general formula RCN. Further examples of fatty nitriles are found under the classification Fatty nitriles [FA09].

Fatty ethers are lipids with ether bonds to long-chain alkyl moieties in addition to having ester bonds to fatty acids. Examples of Fatty ether may be found under the LIPID MAPS® Lipidomics Gateway classification: Fatty ethers [FA10].

Examples of hydrocarbons suitable for fatty acyls may be found under the LIPID MAPS® Lipidomics Gateway classification: Hydrocarbons [FA11].

Examples of oxygenated hydrocarbons suitable for fatty acyls may be found under the LIPID MAPS® Lipidomics Gateway classification: Oxygenated hydrocarbons [FA12].

Fatty acyl glycosides are a combination of a sugar such as glucose with a fatty alcohol. Preferably, the Fatty acyl glycosides [FA13] are selected from the group consisting of: Fatty acyl glycosides of mono- and disaccharides [FA1301], Sophorolipids [FA1302], Rhamnolipids [FA1303], and Other Fatty acyl glycosides [FA1300].

Examples of other types of fatty acyls may be found under the LIPID MAPS® Lipidomics Gateway classification: Other Fatty Acyls [FA00].

### Glycerolipids (GL

Preferably, the Monoradylglycerols [GL01] are selected from the group consisting of: Monoacylglycerols [GL0101], Monoalkylglycerols [GL0102], and Mono-(1Z-alkenyl)-glycerols [GL0103].

Preferably, the Diradylglycerols [GL02], are selected from the group consisting of: Diacylglycerols [GL201], 1-alkyl,2-acylglycerols [GL202], 1-acyl,2-alkylglycerols [GL0207], Dialkylglycerols [GL0203], 1Z-alkenylacylglycerols [GL0204], Di-glycerol tetraethers [GL0205], and Di-glycerol tetraether glycans [GL0206].

Preferably, the Triradylglycerols [GL03] are selected from the group consisting of: Triacylglycerols [GL0301], Alkyldlacylglycerols [GL0302], Dialkylmonoacyiglycerols [GL303], 1Z-alkenyldiacylglycerols [GL304], and Estolides [GL0305].

Preferably, the Glycosylmonoradylglycerols [GL04] are selected from the group consisting of: Glycosylmonoacylglycerols [GL040], and Glycosylmonoalkylglycerols [GL402].

Preferably, the Glycosyldiradylglycerols [GL05] are selected from the group consisting of: Glycosyldiacylglycerols [GL501], Glycosylalkylacylglycerols, [GL0502], and Glycosyldlalkylglycerols [GL0503].

Examples of other types of glycerolipids may be found under the LIPID MAPS® Lipidomics Gateway classification: Other Glycerolipids [GL00].

### Glycerophosphopids [GP]

Preferably, the Glycerophosphocholines [GP01] are selected from the group consisting of: Diacylglycerophosphocholines [GP0101], 1-alkyl,2-acylglycerophosphocholines [GP0102], 1-acyl,2-alkylglycerophosphocholines [GP0108], 1-(1Z-alkenyl),2-acylglycerophosphocholines [GP0103], Dialkylglycerophosphocholines [GP0104], Monoacylglycerophosphocholines [GP0105], Monoalkylglycerophosphocholines [GP0106], and 1Z-alkenylglycerophosphocholines [GP0107].

Preferably, the Glycerophosphoethanolamines [GP02] are selected from the group consisting of: Diacylglycerophosphoethanolamines [GP0201], 1-alkyl,2-acylglycerophosphoethanolamines [GP0202], 1-acyl,2-alkylglycerophosphoethanolamines [GP0208], 1-(1Z-alkenyl),2-acylglycerophosphoethanolamines [GP0203], Dialkylglycerophosphoethanolamines [GP0204], Monoacylglycerophosphoethanolamines [GP0205], Monoalkylglycerophosphoethanolamines [GP0206], and 1Z-alkenylglycerophosphoethanolamines [GP0207].

Preferably, the Glycerophosphoserines [GP03] are selected from the group consisting of: Diacylglycerophosphoserines [GP0301], 1-alkyl,2-acylglycerophosphoserines [GP0302], 1-(1Z-alkenyl),2-acylglycerophosphoserines [GP0303], Dialkylglycerophosphoserines [GP0304], Monoacylglycerophosphosennes [GP0305], Monoalkylglycerophosphoserines [GP0306] and 1Z-alkenylglycerophosphoserines [GP0307].

Preferably, the Glycerophosphoglycerols [GP04] are selected from the group consisting of: s Diacylglycerophosphoglycerols [GP401], 1-alkyl,2-acylglycerophosphoglycerols [GP402], 1-acyl,2-alkylglycerophosphoglycerols [GP0411], 1-(1Z-alkenyl),2-acylglycerophosphoglycerols [GP0403], Dialkylglycerophosphoglycerols [GP0404, Monoacylglycerophosphoglycerols [GP0405], Monoalkylglycerophosphoglycerols [GP406], 1Z-alkenylglycerophosphoglycerols [GP0407], Diacylglycerophosphodiradylglycerols [GP0408], Diacylglycerophosphomonoradylglycerols GP0409], and Monoacylglycerophosphomonoradylglycerols [GP0410].

Preferably, the Glycerophosphoglycerophosphates [GP05] are selected from the group consisting of: Diacylglycerophosphoglycerophosphates [GP0501], 1-alkyl,2-acylglycerophosphoglycerophosphates [GP0502], 1-(1Z-alkenyl),2-acylglycerophosphoglycerophosphates [GP0503], Dialkylglycerophosphoglycerophosphates [GP0504], Monoacylglycerophosphoglycerophosphates [GP0505], Monoalkyiglycerophosphoglycerophosphates [GP0506], and 1Z-alkenylglycerophosphoglycerophosphates [GP0507].

Preferably, the Glycerophosphoinositols [GP06] are selected from the group consisting of: Diacylglycerophosphoinositols [GP0601], 1-alkyl,2-acylglycerophosphoinositols [GP0602], 1-(1Z-alkenyl),2-acylglycerophosphoinositols [GP0603], Dialkylglycerophosphoinositols [GP0604], Monoacylglycerophosphoinositols [GP0605], Monoalkylglycerophosphoinositols [GP0606], and 1Z-alkenylglycerophosphoinositols [GP0607].

Preferably, the Glycerophosphoinositol monophosphates [GP07] are selected from the group consisting of: Diacylglycerophosphoinositol monophosphates [GP0701], 1-alkyl,2-acylglycerophosphoinositol monophosphates [GP0702], 1-(1Z-alkenyl),2-acylglycerophosphonositol monophosphates [GP0703], Dialkylglycerophosphoinositol monophosphates [GP0704], Monoacylglycerophosphoinositol monophosphates [GP0705], Monoalkylglycerophosphoinositol monophosphates [GP0706], and 1Z-alkenylglycerophosphoinositol monophosphates [GP0707].

Preferably, the Glycerophosphoinositol bisphosphates [GP08] are selected from the group consisting of: Diacylglycerophosphoinositol bisphosphates [GP0801], 1-alkyl,2-acylglycerophosphoinositol bisphosphates [GP0802], 1-(1Z-alkenyl),2-acylglycerophosphoinositol bisphosphates [GP0803], Monoacylglycerophosphoinositol bisphosphates [GP0804], Monoalkylglycerophosphoinositol bisphosphates [GP0805], and 1Z-alkenylglycerophosphoinositol bisphosphates [GP0806].

Preferably, the Glycerophosphoinositol trisphosphates [GP09] are selected from the group consisting of: Diacytglycerophosphoinositol trisphosphates [GP0901], 1-alkyl,2-acylglycerophosphoinositol trisphosphates [GP0902], 1-(1Z-alkenyl),2-acylglycerophosphoinositol trisphosphates [GP0903], Monoacylglycerophosphoinositol trisphosphates [GP0904], Monoalkylglycerophosphinositol trisphosphates [GP0905] and 1Z-alkenylglycerophosphoinositol trisphosphates [GP0906].

Preferably, the Glycerophosphates [GP10] are selected from the group consisting of: Diacylglycerophosphates [GP1001], 1-alkyl,2-acylglycerophosphates [GP1002], 1-(1Z-alkenyl),2-acylglycerophosphates [GP1003], Dialkyglycerophosphates [GP1004], Monoacylglycerophosphates [GP1005], Monoalkylglycerophosphates [GP1006], and 1Z-alkenylglycerophosphates [GP1007].

Preferably, the Glyceropyrophosphates [GP11] are selected from the group consisting of: Diacylglyceropyrophosphates [GP1101], and Monoacylglyceropyrophosphates [GP1102].

Preferably, the Glycerophosphoglycerophosphoglycerols [GP12] are selected from the group consisting of: Diacylglycerophosphoglycerophosphodiradtgtycerols [GP1201], Diacylglycerophosphoglycerophosphomonoradylglycerols [GP1202], 1-alkyl,2-acylglycerophosphoglycerophosphodiradyglycerols [GP1203], 1-alkyl,2-acylglycerophosphoglycerophosphomonoradylglycerolS [GP1204], 1-(1Z-alkenyl),2-acylglycerophosphoglycerophosphodiradylglycerols [GP1205], 1-(1Z-alkenyl),2-acylglycerophosphoglycerophosphomonoradylglycerols [GP1206], Dialkylglycerophosphoglycerophosphodiradylglycerols [GP1212], Dialkylglycerophosphoglycerophosphomonoradylglycerols [GP1213], Monoacylglyerophosphoglycerophosphomonoradyglycerols [GP1207], Monoalkylglycerophosphoglycerophosphodiradylglycerols [GP1208], Monoalkyglycerophosphoglycerophosphomonoradylglycerols [GP1209], 1Z-alkenylglycerophosphoglycerophosphodradylglycerols [GP1210], and 1Z-alkenylglycerophosphoglycerophosphomonoradylglycerols [GP1211].

Preferably, the CDP-Glycerols [GP13] are selected from the group consisting of: CDP-diacylglycerols [GP1301], CDP-1-alkyl,2-acylglycerols [GP1302], CDP-1-(1Z-alkenyl),2-acylglycerols [GP1303], CDP-Dialkylglycerols [GP1304], CDP-Monoacylglycerols [GP1305], COP-Monoalkyiglycerols [GP1306], and CDP-1Z-alkenylglycerols [GP1307].

Preferably, the Glycosylglycerophospholipids [GP14] are selected from the group consisting of: Diacylglycosylglycerophospholipids [GP1401], 1-alkyl,2-acyglycosyglycerophospholipids [GP1402], 1-(1Z-alkenyl),2-acylglycosylglycerophospholipids [GP1403], Dialkylglycosylglycerophospholipids [GP1407], Monoacylglycosylglycerophospholipids [GP1404], Monoalkylglycosylglycerophospholipids [GP1405], and 1Z-alkenylglycosylglycerophospholipids [GP1406].

Preferably, the Glycerophosphoinositolglycans [GP15] are selected from the group consisting of, Diacylglycerophosphoinositolglycans [GP1501], 1-alkyl,2-acylglycerophosphoiniositolglycans [GP1502], 1-(1Z-alkenyl),2-acylglycerophosphoinositolglycans [GP1503], Dialkylglycerophospholnositolglycans [GP1507], Monoacylglycerophosphoinositolglycans [GP1504], Monoalkylglycerophosphomnositolglycans [GP1505], and 1Z-alkenylglycerophosphoinositolglycans [GP1506].

Preferably, the Glycerophosphonocholines [GP16] are selected from the group consisting of: Diacylglycerophosphonocholines [GP1601], 1-alkyl,2-acylglycerophosphonocholines [GP1602], 1-(1Z-alkenyl),2-acylglycerophosphonocholines [GP1603], Dialkylglycerophosphonocholines [GP1604], Monoacylglycerophosphonocholines [GP1605], Monoalkylglycerophosphonocholines [GP1606], and 1Z-alkenylglycerophosphonocholines [GP1607].

Preferably, the Glycerophosphonoethanolamines [GP17] are selected from the group consisting of: Diacylglycerophosphonoethanolamines [GP1701], 1-alkyl,2-acylglycerophosphonoethanolamines [GP1702], 1-(1Z-alkenyl),2-acylglycerophosphonoethanolamines [GP1703], Dialkylglycerophosphonoethanolamines [GP1704], Monoacylglycerophosphonoethanolamines [GP1705], Monoalkylglycerophosphonoethanolamines [GP1706], and 1Z-alkcenylglycerophosphonoethanolamines [GP1707].

Examples of Di-glycerol tetraether phospholipids may be found under the LIPID MAPS® Lipidomics Gateway classification: Di-glycerol tetraether phospholipids (caldarchaeols) [GP18];

Examples of Glycerol-nonitol tetraether phospholipids may be found under the LIPID MAPS® Lipidomics Gateway classification: Glycerol-nonitol tetraether phospholipids [GP19];

Preferably, the Oxidized glycerophospholipids [GP20] are selected from the group consisting of: Oxidized glycerophosphocholines [GP2001], Oxidized glycerophosphoethanolamines [GP2002], and Oxidized Cardiolipins [GP2003].

Examples of Glycerol-nonitol tetraether phospholipids may be found under the LIPID MAPS® Lipidomics Gateway classification: Other Glycerophospholipids [GP00];

### Sphingolipids [SP]

Preferably, the Sphingoid bases [SP01] are selected from the group consisting of: Sphing-4-enines (Sphingosines) [SP0101], Sphinganines [SP0102], 4-Hydroxysphinganines (Phytosphingosines) [SP0103], Sphingoid base homologs and variants [SP0104], Sphingoid base 1-phosphates [SP0105], Lysosphingomyelins and lysoglycosphingolipids [SP0106], N-methylated sphingoid bases [SP0107], and Sphingoid base analogs [SP0108].

Preferably, the Ceramides [SP21] are selected from the group consisting of: N-acylsphingosines (ceramides) [SP0201], N-acylsphinganines (dihydroceramides) [SP0202], N-acyl-4-hydroxysphinganines (phytoceramides) [SP203], Acylceramides [SP0204], and Ceramide 1-phosphates [SP0205].

Preferably, the Phosphosphingolipids [SP03] are selected from the group consisting of: Ceramide phosphocholines (sphingomyelins) [SP0301], Ceramide phosphoethanolamines [SP0302], and Ceramide phosphoinositols [SP0303].

### Phosphonosphingolipids [SP04]

Preferably, the Neutral glycosphingolipids [SP05] are selected from the group consisting of: Simple Glc series [SP0501], GalNAcβ1-3Galα1-4Galβ1-4Glc- (Globo series) [SP0502], GalNAcβ1-4Galβ1-4Glc- (Ganglio series) [SP0503], Galβ1-3GlcNAcβ1-3Galβ1-4Glc- (Lacto series) [SP0504], Galβ1-4GlcNAcβ1-3Galβ1-4Glc- (Neolacto series) [SP0505], GalNAcβ1-3Galα-3Galβ1-4Glc- (Isoglobo series) [SP0506], GlcNAcβ1-2Manα1-3Manβ1-4Glc- (Mollu series) [SP0507], GalNAcβ1-4GlcNAcβ1-3Manβ1-4Glc- (Arthro series) [SP0508], Gal- (Gala series) [SP0509], and Other Neutral glycosphingolipids [SP0500].

Preferably, the Acidic glycosphingolipids [SP06] are selected from the group consisting of: Gangliosides [SP0601], Sulfoglycosphingolipids (sulfatides) [SP0602], Glucuronosphingolipids [SP0603], Phosphoglycosphingolipids [SP0604], and Other Acidic glycosphingolipids [SP0600].

Examples of Basic glycosphingolipids may be found under the LIPID MAPS® Lipidomics Gateway classification: Basic glycosphingolipids [SP07].

Examples of Amphoteric glycosphingolipids may be found under the LIPID MAPS® Lipidomics Gateway classification: Amphoteric glycosphingolipids [SP08].

Examples of Arsenosphingolipids may be found under the LIPID MAPS® Lipidomics Gateway classification: Arsenosphingolipids [SP09].

Examples of Other Sphingolipids may be found under the LIPID MAPS® Lipidomics Gateway classification: Other Sphingolipids [SP00].

### Sterol Lipids [ST]

Preferably, the Sterols [ST01] are selected from the group consisting of: Cholesterol and derivatives [ST0101], Steryl esters [ST0102], Ergosterols and C24-methyl derivatives [ST0103], Stigmasterols and C24-ethyl derivatives [ST0104], C24-propyl sterols and derivatives [ST0105], Gorgosterols and derivatives [ST0106], Furostanols and derivatives [ST0107], Spirostanols and derivatives [ST0108], Furospirostanols and derivatives [ST0109], Cycloartanols and derivatives [ST0110], Calysterols and cyclopropyl sidechain derivatives [ST0111], Cardanolides and derivatives [ST0112], Bufanolides and derivatives [ST0113], Brassinolides and derivatives [ST0114], Solanidines and alkaloid derivatives [ST0115], and Withanolides and derivatives [ST0116]. For example, Sterol lipids include cholesterol, and phytosterol.

Preferably, the Steroids [ST02] are selected from the group consisting of: C18 steroids (estrogens) and derivatives [ST0201], C19 steroids (androgens) and derivatives [ST0202], and C21 steroids (gluco/mineralocorticoids, progestogins) and derivatives [ST0203].

Preferably, the Secosteroids [ST03] are selected from the group consisting of: Vitamin D2 and derivatives [ST0301], Vitamin D3 and derivatives [ST0302], Vitamin D4 and derivatives [ST0303], Vitamin D5 and derivatives [ST0304], Vitamin D6 and derivatives [ST0305], and Vitamin D7 and derivatives [ST0306].

Preferably, the Bile acids and derivatives [ST04] are selected from the group consisting of: C24 bile acids, alcohols, and derivatives [ST0401], C26 bile acids, alcohols, and derivatives [ST0402], C27 bile acids, alcohols, and derivatives [ST0403], C28 bile acids, alcohols, and derivatives [ST0404], C22 bile acids, alcohols, and derivatives [ST0405], C23 bile acids, alcohols, and derivatives [ST0406], C25 bile acids, alcohols, and derivatives [ST0407], and C29 bile acids, alcohols, and derivatives [ST0408].

Preferably, the Steroid conjugates [ST05] are selected from the group consisting of: Glucuronides [ST0501], Sulfates [ST0502], Glycine conjugates [ST0503], Taurine conjugates [ST0504], and Other Steroid conjugates [ST0505].

More suitably still, the lipophilic tail component of the anti-agglomerant as described herein may be derived from the group of oil soluble lipid based products consisting of: alkaloids, terpenes, terpenoids, flavonoids, glycosides, natural phenols, phenazines, biphenyls, dibenzofurans, polyketides, fatty acid synthase products, nonribosomal peptides, ribosomally synthesised and post-translationally modified peptides, natural polyphenols, vitamins, and steroids.

Steroid based products are particularly preferred and include, for example, C17 steroids such a gonane, C18 steroids such estrane, dehydrogenated C18 steroids such estrene, estradiene, estratriene, substituted C18 steroids including estratrienolone, estratrienolone, estratrienediol, estratrienetriol, C19 steroids, such as 19-norpregnane, Androstane: Dehydrogenated C19 steroids such as Androstene, Androstadiene, Androstatriene; Substituted C19 steroids such as Androstanol, Androstenol, Androstanone, Androstenone, Androstanediol, Androstenediol, Androstanedione, Androstenedione, Androstenetrione, Androstanolone. Androstenolone, Androstadienol, Androstadienone; Etiocholane, C21 steroids such as Pregnane: Dehydrogenated: Pregnene, Pregnadiene, Pregnatriene: Substituted: Prognanediol, Pregnanetriol, Pregnenediol, Pregnanedione, Pregnenedione, C23 steroids such as cardanolide, C24 steroids such as chloane, bile acid, C27 steroids such as cholestane. Examples of 17-ketosteroids include: Androstenedione, Androstanedione, Androsterone, Dehydroepiandrosterone. Epiandrosterone, Epietiocholanolone, Estrone, Etiocholanolone, Hydroxysterold, halogenated steroids. Norsteroids, Secosteroids such as cholecalciferol (vit D3), Azasteroids such as finasteride and dutasteride. Steranes derived from sterol and constitutes the core of all sterols, including Cholesterol and its derivatives (such as progesterone, aldosterone, cortisol, and testosterone.

### Prenol Lipids [PR]

Prenol lipids include terpenoids derived from monocyclic terpenes include menthol, thymol, carvacrol; monocyclic sesqueterpene such as humulene, beta-caryophyllene, Bicyclic monoterpenes such as carene, sabinene, camphene, thujene; bicyclic monoterpenoids camphor, bomeol, eucalyptol. Monocyclic sesqueterpene such as zingiberene. Bicyclic sesqueterpene include caryophyllene, vetivazulene, guaiazulene. Tricyclic sesqueterpenes such as longitolene, copaene and the alcohol patchoulol. Hemiterpenes (isoprene) and hemiterpenoid (prenol and isovaleric acid); monoterpene and monoterpenoids (2 isoprene units, geraniol, terpineol, limonene, myrcene, linalool, pinene, iridoids); sesquiterpenes and sesquiterpenoids (3 isoprene units, shumulene, farnesenes, farnesol), diterpenes and diterpenoids (4 isoprene units, cafestol, kahweol, cembrene, taxadiene, and form basis for retinol, retinal and phytol (precursor to Vitamin E and Vitamin K1, chlorophyll)), sesterterpenes and sesqueterpenoids (7 isoprene units) including ferrugucadiol and tetraprenylcurcumene), triterpenes and triterpenoids (6 isoprene units, squalene precursors to lansterol or cycleartenol), and tetraterpenes and tetraterpenoids (8 isoprene units, and include lycopene, gamma-carotene, and alpha and beta carotene) and norisoprenoids. Terpene hydrocarbon based compounds including: isoprene include phytol, retinol (vit A), tocopherol, dolichol, squalene, lanosterol, carotenoids, and quinines. Terepenoids which contain additional functional groups to the corresponding terpene or have rearranged skeletons making them isoterpenes, for example vitamin A. Diterpenoid include gibberellin plant hormones, including gibberellic acid, triterpenes, particularly squalene derivatives which form the basis for steroids having a curcurbitane core and include sterols and curcurbitacins or steroid precursors such as lanosterol or cycloartenol, as well as triterpenoid derivatives such as triterpenoid glycosides such as triterpenoid saponins which include ginsenosides, astragaloside, and eleutheroides. Tetraterpenes for example, phytoene, and tetraterpenoids such as carotenes and carotenoids and xanthophylls. Carotenoids such as xanthophylls which include lutein, zeaxanthin, neoxanthin, violaxanthin, anterazanthin, diadinoxanthin, diatoxantin, zeaxanthin, dinoxanthin, flavoxanthin, alpha and beta-cryptoxanin, Antheraxanthin, Astaxanthin, Canthaxanthin, Citranaxanthin, Cryptoxanthin, Diadinoxanthin, Diatoxanthin, Dinoxanthin, Echinenone, Flavoxanthin, Fucoxanthin, Lutein, Neoxanthin, Rhodoxanthin, Rubixanthin, Violaxanthin, Zeaxanthin, carotenoid derivatives apocarotenoids such as Abscisic acid, Apocarotenal, Bixin, Crocetin, Food orange 7, Ionones, Peridinin. Carotenes such as α-Carotene, β-Carotene, γ-Carotene, δ-Carotene, ε-Carotene, ζ-Carotene, Lycopene, Neurosporene, Phytoene, Phytofluene, Torulene, Lycopersene. Vitamin A retinoids such as retinal, retinoic acid, retinol. Well-known terpenoids include citral, geranial, neral, menthol, camphor, salvinorin A, cannabinoids, ginkgolide and bilobalide.

Preferably, the Isoprenoids [PR01] are selected from the group consisting of: C5 isoprenoids (hemiterpenes) [PR0101]; C10 isoprenoids (monoterpenes) [PR0102] including Acyclic monoterpenoids [PR010201], Irregular acyclic monoterpenoids [PR010202], Halogenated dimethyloctane monoterpenoids [PR010203], Ochtodane monoterpenoids [PR010204], Ethyl, dimethylcyclohexane monoterpenoids [PR010205], Cyclopropane and cyclobutane monoterpenoids [PR010206], Iridoid, 10-alkyliridoid and secoiridoid monoterpenoids [PR010207], Other cyclopentane monoterpenoids [PR010208], Menthane monoterpenoids [PR010209], Other cyclohexane monoterpenoids [PR010210], Cycloheptane monoterpenoids [PR010211], Bicyclic monoterpenoids [PR010212], Tricyclic monoterpenoids [PR010213], Other monoterpenoids [PR010214]; C15 isoprenoids (sesquiterpenes) [PR0103] including Acyclic farnesane sesquiterpenoids [PR010301] Furanoid farnesane sesquiterpenoids [PR010302], Irregular acyclic sesquiterpenoids [PR010303], Cyclobutane and cyclopentane sesquiterpenoids [PR010304], Cyclofarnesane sesquiterpenoids [PR010305], Bisabolane sesquiterpenoids [PR010306], Cyclobisabolane sesquiterpenoids [PR010307], Elemane sesquiterpenoids [PR010308], Germacrane sesquiterpenoids [PR010309], Lepidozanes and bicyclogermacrane sesquiterpenoids [PR010310], Humulane sesquiterpenoids [PR010311], Caryophyllane sesquiterpenoids [PR010312], Bicyclohumulane sesquiterpenoids [PR010313], Cuparane sesquiterpenoids [PR010314], Cyclolaurane sesquiterpenoids [PR010315], Herbertane sesquiterpenoids [PR010316], Laurane sesquiterpenoids [PR010317], Trichothecane sesquiterpenoids [PR010318], Eudesmane sesquiterpenoids [PR010319], Emmotin sesquiterpenoids [PR010320], Oppositane sesquiterpenoids [PR010321], Farfugin sesquiterpenoids [PR010322], Cycloeudesmane sesquiterpenoids [PR010323], Gorgonane sesquiterpenoids [PR010324], Eremophilane sesquiterpenoids [PR010325], Chiloscyphane sesquiterpenoids [PR010326], Aristolane sesquiterpenoids [PR010327], Nardosinane sesquiterpenoids [PR010328], Brasilane sesquiterpenoids [PR010329], Cacalol sesquiterpenoids [PR010330], Valerane sesquiterpenoids [PR010331], Rearranged eudesmane sesquiterpenoids [PR010332], Cadinane sesquiterpenoids [PR010333], Alliacane sesquiterpenoids [PR010334], Oplopane sesquiterpenoids [PR010335], Mutisianthol sesquiterpenoids [PR010336], Drimane sesquiterpenoids [PR010337], Coloratane sesquiterpenoids [PR010338], Xanthane sesquiterpenoids [PR010339], Carabrane sesquiterpenoids [PR010340], Guaiane sesquiterpenoids [PR010341], Pseudoguaiane sesquiterpenoids [PR010342], Aromadendrane sesquiterpenoids [PR010343], Cubebane and ivaxillarane sesquiterpenoids [PR010344], Patchoulane sesquiterpenoids [PR010345], Valerenane sesquiterpenoids [PR010346], Africanane sesquiterpenoids [PR010347], Lippifoliane and himachalane sesquiterpenoids [PR010348], Longipinane sesquiterpenoids [PR010349], Longifolane sesquiterpenoids [PR010350], Longibomane sesquiterpenoids [PR010351], Pinguisane sesquiterpenoids [PR010352], Thapsane and fukinane sesquiterpenoids [PR010353], Picrotoxane sesquiterpenoids [PR010354], Daucane sesquiterpenoids [PR010355], Isodaucane sesquiterpenoids [PR010356], Perforane and pacifigorgiane sesquiterpenoids [PR010357], Asteriscane sesquiterpenoids [PR010358], Illudane and protoilludane sesquiterpenoids [PR010359], Sterpurane sesquiterpenoids [PR010360], Illudalane sesquiterpenoids [PR010361], Isolactarane, merulane, lactarane and marasmane sesquiterpenoids [PR010362], Furodysin and furodysinin sesquiterpenoids [PR010363], Botrydial sesquiterpenoids [PR010364], Spirovetivane sesquiterpenoids [PR010365], Acorane sesquiterpenoids [PR010366], Chamigrane sesquiterpenoids [PR010367], Spirosesquiterpenoids [PR010368], Cedrane and isocedrane sesquiterpenoids [PR010369], Zizaane and prezizaane sesquiterpenoids [PR010370], Clovane sesquiterpenoids [PR010371], Precapnellane and capnellane sesquiterpenoids [PR010372], Hirsutane and rearranged hisutane sesquiterpenoids [PR010373], Pentalenane sesquiterpenoids [PR010374], Silphinane, sllphiperfoliane and presilphiperfoliane sesquiterpenoids [PR010375], Isocomane sesquiterpenoids [PR010376], Panasinsane sesquiterpenoids [PR010377], Modhephane sesquiterpenoids [PR010378], Quadrane sesquiterpenoids [PR010379], Campherenane and santalane sesquiterpenoids [PR010380], Sativane sesquiterpenoids [PR010381], Copacamphane and sinularane sesquiterpenoids [PR010382], Copaane sesquiterpenoids [PR010383], Ishwarane sesquiterpenoids [PR010384], Rotundane sesquiterpenoids [PR010385], Thujopsane sesquiterpenoids [PR010386], Bourbonane sesquiterpenoids [PR010387], Gymnomitrane sesquiterpenoids [PR010388], Other sesquiterpenoids [PR010389];

C20 isoprenoids (diterpenes) [PR0104] including Acyclic diterpenoids [PR010401], Prenylbisabolane and cyclophytane diterpenoids [PR010402], Labdane and halimane diterpenoids [PR010403], Colensane and clerodane diterpenoids [PR010404], Abietane diterpenoids [PR010405], Cycloaibetiane and Abeoabietaine diterpenoids [PR010406], Totarane and nagilactone diterpenoids [PR010407], Pimarane diterpenoids [PR010408], Cassane and vouacapane diterpenoids [PR010409], Cleistanthane and isocleistanthane diterpenoids [PR010410], Isocopalane and spongiane diterpenoids [PR010411], Podocarpane diterpenoids [PR010412], Kaurane and phyllocladane diterpenoids [PR010413], Beyerane diterpenoids [PR010414], Villanovane, atisane, trachylobane and helvifulvane diterpenoids [PR010415], Aphidicolane diterpenoids [PR010416], Gibberellins [PR010417], Leucothol and grayanotoxane diterpenoids [PR010418], Cembrane diterpenoids [PR010419], Rearranged cembrane diterpenoids [PR010420], Eunicellane and asbestinane diterpenoids [PR010421], Sphaerane diterpenoids [PR010422], Briarane diterpenoids [PR010423], Dolabellane and modified dolabellane diterpenoids [PR010424], Dolastane and modified dolastane diterpenoids [PR010425], Cyathane diterpenoids [PR010426], Sphaeroane diterpenoids [PR010427], Verrucosane and modified verrucosane diterpenoids [PR010428], Casbane diterpenoids [PR010429], Jatrophane and cyclojatrophane diterpenoids [PR010430], Lathyrane diterpenoids [PR010431], Rhamnofolane and daphnane diterpenoids [PR010432], Tigliane and ingenane diterpenoids [PR010433], Jatropholane and secojatropholane diterpenoids [PR010434], Fusicoccane diterpenoids [PR010435], Valparane and mulinane diterpenoids [PR010436], Spatane diterpenoids [PR010437], Verticillane diterpenoids [PR010438], Taxane and Abeotaxane diterpenoids [PR010439], Trinervitane and kempane diterpenoids [PR010440], Amphilectane, cycloamphilectane, adoclane and neoamphilectane diterpenoids [PR010441], Xenicane and xeniaphyllane diterpenoids [PR010442], Viscidane diterpenoids [PR010443], Eremane diterpenoids [PR010444], Prenyeudesmane, prenylgermacrane and prenylbicyclogermacrane diterpenoids [PR010445], Lobane diterpenoids [PR010446], Pachydictyane and cneorubin diterpenoids [PR010447], Serrulatane and biflorane diterpenoids [PR010448], Decipiane diterpenoids [PR010449], Sacculatane diterpenoids [PR010450], Obtusane diterpenoids [PR010451], Irieol diterpenoids [PR010452], Sphenolobane diterpenoids [PR010453], Ginkgolides and Bilobalides [PR010454]. Other diterpenoids [PR010455];
C25 Isoprenoids (sesterterpenes) [PR0105] including Acyclic sesterterpenoids [PR010501], Cyclohexane sesterterpenoids [PR010502], Cericerane sesterterpenoids [PR010503], Bicyclic sesterterpenoids [PR010504], Cheilanthane and ophiobolane sesterterpenoids [PR010505], Scalarane sesterterpenoids [PR010506], Other sesterterpenoids [PR010507];
C30 isoprenoids (triterpenes) [PR0106] including Acyclic triterpenoids [PR010601], Cyclohexane triterpenoids [PR010602], Botryococcene triterpenoids [PR010603], Prostostane and fusidane triterpenoids [PR010604], Lanostane triterpenoids [PR010605], Cycloartane triterpenoids [PR010606], Cucurbitane triterpenoids [PR010607], Dammarane triterpenoids [PR010608], Tirucallaneleuphane triterpenoids [PR010609], Apotirucallane triterpenoids [PR010610], Nortriterpenoids [PR010611], Quassinoid nortriterpenoids [PR010612], Baocharane triterpenoids [PR010613], Lupane triterpenoids [PR010614], Oleanane triterpenoids [PR010615], Taraxerane, multiflorane, glutinane and friedelane triterpenoids [PR010616], Pachysanane triterpenoids [PR010617], Taraxastane, ursane and bauerane triterpenoids [PR010618], Hopane triterpenoids [PR010619], Neohopane, fernane, adianane and filicane triterpenoids [PR010620], Arborinane and stictane triterpenoids [PR010621], Gammacerane triterpenoids [PR010622], Serratane and onocerane triterpenoids [PR010623], Polypodane, malabaricane and podiodane triterpenoids [PR010624], and Other triterpenoids [PR010625].

Examples of C40 isoprenoids may be found under the LIPID MAPS® Lipidomics Gateway classification: C40 isoprenoids (tetraterpenes) [PR0107]; Examples of polyterpene may be found under the LIPID MAPS® Lipidomics Gateway classification: Polyterpenes [PR0108]; Examples of retinoids may be found under the LIPID MAPS® Lipidomics Gateway classification: Retinoids [PR0109]; Preferably, the Quinones and hydroquinones [PR02] are selected from the group consisting of: Ubiquinones [PR0201], Vitamin E [PR0202], and Vitamin K [PR0203].

Preferably, the Polyprenols [PR03] are selected from the group consisting of: Bactoprenols [PR0301], Bactoprenol monophosphates [PR0302], Bactoprenol diphosphates [PR0303], Phytoprenols [PR0304], Phytoprenol monophosphates [PR0305], Phytoprenol diphosphates [PR0306], Dolichols [PR0307], Dolichol monophosphates [PR0308], and Dolichol diphosphates [PR0309].

Examples of hopanoids may be found under the LIPID MAPS® Lipidomics Gateway classification: Hopanoids [PR04].

### Saccharolipids [SL]

Preferably, the Acylaminosugars [SL01] are selected from the group consisting of: Monoacylaminosugars [SL0101], Diacylaminosugars [SL0102], Triacylaminosugars [SL0103], Tetraacylaminosugars [SL0104], Pentaacylaminosugars [SL0105], Hexaacylaminosugars [SL0106], and Heptaacylaminosugars [SL0107].

Examples of Acylaminosugar glycans may be found under the LIPID MAPS® Lipidomics Gateway classification: Acylaminosugar glycans [SL02].

Examples of Acyltrehaloses may be found under the LIPID MAPS® Lipidomics Gateway classification: Acyltrehaloses [SL03].

Examples of Acyltrehalose glycans may be found under the LIPID MAPS® Lipidomics Gateway classification: Acyltrehalose glycans [SL04].

Examples of Other acyl sugars may be found under the LIPID MAPS® Lipidomics Gateway classification: Other acyl sugars [SL05].

Examples of Other Saccharolipids may be found under the LIPID MAPS® Lipidomics Gateway classification: Other Saccharolipids [SL00].

### Polyketides [PK]

The polyketides span type I polyketides, type II polyketides, type II polyketides and include macrolides, ansamycins, polyenes, polyethers, tetracyclines, and acetogenins.

Examples of Linear polyketides may be found under the LIPID MAPS® Lipidomics Gateway classification: Linear polyketides [PK01].

Examples of Halogenated acetogenins may be found under the LIPID MAPS® Lipidomics Gateway classification: Halogenated acetogenins [PK02].

Examples of Annonaceae acetogenins may be found under the LIPID MAPS® Lipidomics Gateway classification: Annonaceae acetogenins [PK03].

Examples of Macrolides and lactone polyketides may be found under the LIPID MAPS® Lipidomics Gateway classification: Macrolides and lactone polyketides [PK04].

Examples of Ansamycins and related polyketides may be found under the LIPID MAPS® Lipidomics Gateway classification: Ansamycins and related polyketides [PK05].

Examples of Polyenes may be found under the LIPID MAPS® Lipidomics Gateway classification: Polyenes [PK06].

Examples of Linear tetracyclines may be found under the LIPID MAPS® Lipidomics Gateway classification: Linear tetracyclines [PK07].

Examples of Angucyclines may be found under the LIPID MAPS® Lipidomics Gateway classification: Angucyclines [PK08].

Examples of Polyether antibiotics may be found under the LIPID MAPS® Lipidomics Gateway classification: Polyether antibiotics [PK09];

Examples of Aflatoxins and related substances may be found under the LIPID MAPS® Lipidomics Gateway classification: Aflatoxins and related substances [PK10];

Examples of Cytochalasins may be found under the LIPID MAPS® Lipidomics Gateway classification: Cytochalasins [PK11].

Preferably, the Flavonoids [PK12], are selected from the group consisting of: Anthocyanidins [PK1201], Flavans, Flavanols and Leucoanthocyanidins [PK1202], Proanthocyanidins [PK1203], Biflavonoids and polyflavonoids [PK1204], Isoflavonoids [PK1205], Rotenoid flavonoids [PK1206], Pterocarpans [PK1207], Isoflavans [PK1208], Coumestan flavonoids [PK1209], Neoflavonoids [PK1210], Flavones and Flavonols [PK1211], Chalcones and dihydrochalcones [PK1212], Aurone flavonoids [PK1213], Flavanones [PK1214], Dihydroflavonols [PK1215], and Other Flavonoids [PK1216].

Preferably, the Aromatic polyketides [PK13] are selected from the group consisting of: Monocyclic aromatic polyketides [PK1301], Naphthalenes and naphthoquinones [PK1302], Benzoisochromanquinones [PK1303], Anthracenes and phenanthrenes [PK1304], Anthracyclinones [PK1305], Dibenzofurans, griseofuvins, dibenzopyrans and xanthones [PK1306], Diphenylmethanes, acylphloroglucinols and benzophenones [PK1307], Depsides and depsidones [PK1308], Diphenyl ethers, biphenyls, dibenzyls and stilbenes [PK1309], Benzofuranoids [PK1310], Benzopyranoids [PK1311], and Other aromatic polyketides [PK1312].

Examples of Non-ribosomal peptide/polyketide hybrids may be found under the LIPID MAPS® Lipidomics Gateway classification: Non-ribosomal peptide/polyketide hybrids [PK14].

Preferably, the Phenolic lipids [PK15] are selected from the group consisting of: Alkyl phenols and derivatives [PK1501], Alkyl catechols and derivatives [PK1502], Alkyl resorcinols and derivatives [PK1503], Anacardic acids and derivatives [PK1504], and Alkyl hydroquinones and derivatives [PK1505].

Examples of Other Polyketides may be found under the LIPID MAPS® Lipidomics Gateway classification: Other Polyketides [PK00].

Other naturally occurring products and derivatives include linear diarylheptanoids including curcuminoids such as desmethoxycurcumin, curcumin or derivatives thereof. Cyclic diarylheptanoids such as myricanone, ostryositrienol, ostryopsitriol, acerogenin M, jugcathayenoside, and galeon. Tetrapyrrole based compounds including linear bilane tetrapyrroles and phytobilins. Linear bilane tetrapyrrole based compounds include bilirubin, biliverdin, stercobilinogen, stercobilin, urobilinogen, and urobilin. Phytobilins include phytochromobilin, and phycobilins include phycocyanobilin, phycourobilin, and Phycoviolobilin. Macrocyclic tetrapyrroles including corrinoids, porphyrins, and reduced porphyrins. Corrinoids including methylcobalamin, adenosylcobalamin, cyanocobalamin, phycobilins, luciferins, and cyclic tetrapyrroles including porphin, chlorins, corrins, corrinoids, porphyrins such as protoporphyrins such as protoporphyrin IX, and heme b, c, a and o, phytoporphyrins such as chlorophyll c and protochlorophyllide, reduced porphyrins such as porphyrinogens, chlorins, bacteriochlorins, isobacteriochlorins, corphins including cofactor F430. Reduced porphyrins include chlorins, corrins, bacteriochlorins, isobacteriochlorins, porphins and corphins. Chlorins include Chlorophyllide (a, b), Chlorophyll (a, b). Phaeophytin (a, b), Bacteriochlorophyll c. Bacteriochlorins include bacteriochlorophyll a. Isobacteriochlorins include Siroheme and Sirohydrochlorin.

Pigments include compounds containing conjugated systems including chlorin based pigments, corrin based pigments, bacteriochlorin pigments, isobacteriochlorins, porphin based pigments, carotenoid based pigments, corphin based pigments, flavanoid pigments, light emitting pigments, melanin pigment, urochrome pigments, polyene enolate. Pigments, phytochrome pigments, phycobiliproteins and derivatives thereof. Betalains, curcuminoids, flavonoids, and carotenoids. Betalains include betacyanins such as betanin, isobetanin, probetanin, and neobetanin; betaxanthins Include vulgaxanthin, miraxanthin, portulaxanthin and indicaxanthin. Porphyrin based pigments include Chlorophylls include chlorophyll a, chlorophyll b, chlorophyll c, chlorophyll d, and chlorophyll f. Curcuminoids such as desmethoxycurcumin, curcumin or derivatives thereof.

Flavanoid pigments include anthocyanins, including anthocyanins, anthocyanidins, betalains anthoxanthins, flavans, and condensed tanins. Carotenoids including carotenes, retinoids, and xanthophylls. Other pigments include allophycocyanin, phycocyanin, phycoerythrin, phycoerythrocyanin, quinones, and xanthonoids. Porphyrin based pigments include chlorophyll, bilirubin, haemocyanin, haemoglobin, and myoglobin. Carotenoid pigments including hematochromes, carotenes, and xanthophylls. Light emitting pigments such as luciferin, bacteriochlorins and isobacteriochlorins. Hematochromes include algal pigments, mixes of carotenoids and their derivatives.

Carotenoids include hematochromes, carotenes, xanthophylls, and retinoids.

Carotenes include alpha and beta carotene, lycopene, rhodopsin. Xanthophylls include canthaxanthin, zeaxanthin, lutein. Proteinaceous pigments include phytochrome, phycobiliproteins, Polyene enolates, Rhodopsin, melanin, eumelanin, pheomelanin, Trichochromes, neuromelanin, urochrome, quinones, xanthonoids.

Alkaloids including capsaicinoids including capsacin, dihydrocapsacin, nordihydrocapsaicin, homocapsaicin, and homodihydrocapsaicin.

Glycoconjugates include glycoproteins, glycopeptides, peptidoglycans, glycolipids, and lipopolysaccharides.

Glycosides selected from the group consisting of: alcoholic glycosides, anthraquinone glycosides, coumarin glycosides, chromone glycosides, cyanogenic glycosides, flavonoid glycosides, phenolic glycosides, saponin, steroidal glycosides, steviol glycosides, iridoid glycosides, and thioglycosides and derivatives thereof. Alcoholic glycosides include salicin. Anthraquinone glycosides, coumarin glycosides such as apterin, chromone glycosides, cyanogenic glycosides such as amygdalin, and prunasin, flavonoid glycosides such as hesperidin, naringin, rutin, quercetin, phenolic glycosides such as arbutin, triterpene glycosides including saponin glycosides such as diosgenin, ginsenosides, steroidal glycosides, steviol glycosides include stevioside and rebaudioside A, iridoid glycosides including aucubin, Geniposidic acid, theviridoside, Loganin, Catalpol, and thioglycosides such as sinigrin and sinalbin, and derivative thereof.

Amino acid derived hormones such as norepinephrine, melatonin and thyroxine. Peptide hormone including small peptide hormones including TRH and vasopressin, oxytocin. Protein hormones such as human growth hormone, luteinizing hormone, follicle stimulating hormone and thyroid stimulating hormone.

Steroid hormones which are typically derived from the lipid cholesterol and include androgens for example, Androstenedione, Dehydroeplandrosterone, Dihydrotestosterone, Testosterone, and estrogens for example estradiol, estriol, estrone and progestoens [progesterone]. Plant hormones include abscisic acid hormone, auxins, cytokinins, and gibberellins. Prostanoids include prostaglandins, thromboxanes, leukotrienes and lipoxins, resolvins, eoxins.

Vitamins include Vitamin A, Vitamin B₁, Vitamin B₂, Vitamin B₃, Vitamin B₄, Vitamin B₅, Vitamin B₆, Vitamin B₇, Vitamin B₉, Vitamin C, Vitamin D, Vitamin E, and Vitamin K. For example, the vitamin A component may be selected from the group of vitamin A compounds consisting of: retinol, retinal, and provitamin A carotenoids, including beta carotene, or vitamin A precursors such as retinyl palmitate. Carotenes including alpha-carotene, gamma-carotene, beta-cryptoxanthin. Vitamin B₁ otherwise known as thiamine. Vitamin B₁₂, is selected from the group consisting of: cydroxocobalamin, methylcobalamin, and adenosylcobalamin. Vitamin B₂, otherwise known as riboflavin. Vitamin B₃ such as niacin, niacinamide, nicotinamide riboside. Vitamin B₆, otherwise known as pantothenic acid. Vitamin B₆ selected from the group consisting of: Pyridoxine, pyridoxamine, pyridoxal. Vitamin B₇, biotin. Vitamin B₉, selected from the group consisting of: folic acid, folinic acid. Vitamin B₁₂, selected from the group consisting of: Cyanocobalamin, hydroxocobalamin, methylcobalamin, adenosylcobalamin. Vitamin C, Ascorbic acid, Vitamin D such Cholecalciferol (D3), Ergocalciferol (D2), Vitamin E selected from the group consisting of: Tocopherols, tocotrienols, Vitamin K, selected from the group consisting of: Phylloquinone, and menaquinones.

Preferably, the lipophilic tail group component is derived from a compound selected from quinones and hydroquinones, for example, Ubiquinones [PR0201], Vitamin E [PR0202], and Vitamin K [PR0203].

In a preferred embodiment, the non-ionic hydrophilic head group component of the anti-agglomerant of the invention is derived from a precursor compound comprising:

a 5- or 6-membered carbocyclic or heterocyclic ring which is not a tetrahydrofuran polyol ring or a fatty ester derivative thereof, and which is substituted with at least one functional group couplable to at least one lipophilic tail group as defined herein.

A polyol is a hydrocarbon based compound comprising more than two hydroxyl or ester groups but no additional functional groups, for example, where three hydroxyls are present the compound is a triol, for example, glycerol, where four hydroxyls are present the compound is a tetrol, etc.

In some embodiments, the 5- or 6-membered carbocyclic or heterocyclic ring is not a tetrahydropuran polyol ring or a fatty ester derivative thereof. Desirably, the 5- or 6-membered carbocyclic or heterocyclic ring is not a saturated cyclic ether ring, particularly those which are substituted with polyols or substituted with polyol derivatives such as fatty acid esters or such rings substituted with mixtures of more than two hydroxyl and polyol derivatives substitutions combined. In other words, saturated rings comprising a cyclic ether 5- or 6-membered ring, particularly polyol, polyol fatty ester derivatives or mixed hydroxyl/hydroxyl fatty ester cyclic ether 5- or 6-membered rings, particularly those substituted with more than two hydroxyl groups or fatty esters thereof, including rings comprising at least one hydroxyl and one fatty ester functionality are excluded as components of the non-ionic head groups and anti-agglomerants of the invention. In some embodiments, unsaturated analogous rings may be excluded.

In some embodiments, cyclitols particularly dehydrated sugar alcohol cyclitols or dehydrated sugar alcohol cyclitol ester rings, for example, which comprise a sorbitan ring, or in some cases, a tetrahydrofuran or tetrahydropuran ring substituted with more than two hydroxyl and/or fatty ester functionalities are excluded. In some embodiments it is preferred that the at least one non-ionic hydrophilic head group is not derived from sorbitol or other sugar polyols such as pentitols or hexitols. Suitably, where the at least one non-ionic hydrophilic head group ring is a polyol, particularly a sugar alcohol, most particularly sorbitan, it is preferred that the lipophilic tail is not derived from octadecane fatty chain in particular. Lauric acid, oleic, palmitic or stearic acid, particularly oleic acid are also less preferred in such embodiments. In particular, the cyclitol sorbitan is excluded when the lipophilic chain component is derived from a fatty chain such as n-octaneoic acid, n-decanoic acid and n-tridecanoic acid. In some embodiments, the 5- or 6-membered carbocyclic or heterocyclic ring is not a cyclic ether ring such as a tetrahydrofuran or a tetrahydropuran ring.

Suitably, the 5- or 6-membered ring may be saturated or unsaturated.

Suitably, the 5- or 6-membered ring is an aromatic carbocyclic ring, an aromatic heterocyclic ring, an aliphatic carbocyclic ring or aliphatic heterocyclic ring provided that the ring is not an excluded ring as described above. Aromatic carbocyclic rings or aromatic heterocyclic rings are preferred. Aromatic carbocyclic rings are particularly preferred.

Examples of suitable aliphatic 5-membered carbocyclic rings include cyclopentane, cyclopentene, and cyclopentadiene. Examples of suitable aliphatic 5-membered heterocyclic rings include tetrahydrofuran subject to the above exclusions, 1,3-dioxolane, tetrahydrothiophene, 1,2-oxathiolane, sulfolane, 2,4-thiazolidinedione, succinimide, 2-oxazolidone, and hydantoin. Examples of suitable aromatic 5-membered heterocyclic rings include furan, thiophene, oxazole, isoxazole, isothiazole, thiazole, 1,2,5-oxadiazole, 1,2,3-oxadiazole, 1,3,4-thiadiazole, and 1,2,5-thiadiazole. Dihydrofuran rings are excluded in embodiments where the ring is substituted with more than two hydroxyl and/or fatty ester groups. Examples of suitable aliphatic 6-membered carbocyclic rings include cyclohexane, cyclohexene, cyclohexadiene. Examples of suitable aliphatic 6-membered heterocyclic rings include piperidine, piperazine, tetrahydropyran subject to the above exclusions, 21-pyran, 4H-pyran, 1,4-dioxane, 1,4-dioxine, thiane, 2H-thiopyran, 4H-thiopyran, 1,3-dithiane, 1,4-dithiane, 1,3,5-trithiane, morpholine, 4H-1,2-oxazine, 2H-1,2-oxazine, 6H-1,2-oxazine, 4H-1,3-oxazine, 2H-1,4-oxazine, thiomorpholine, 4H-1,4-thiazine, 2H-1,2-thiazine, 6H-1,2-thiazine, 2H-1,4-thiazine, cytosine, thyamine, uracilo, thiomorpholine dioxide. An example of an aromatic 6-membered carbocyclic ring is benzene. Examples of suitable aromatic 6-membered heterocyclic rings include pyridine, pyridazine, pyrimidine, pyrazine, and 1,2,3-triazine.

Preferred couplable functional groups on the precursor include amine, carboxylic acid, and hydroxyl functionalities. Particularly preferred couplable functional groups include amine, alkyl amine, alkenyl amine, alkynyl amine, carboxylic acid, alkenyl carboxylic acid, alkynyl carboxylic acid, hydroxyl, alkenyl hydroxyl, or alkynyl hydroxyl. Particularly preferred couplable functional groups are amine, or alkyl amine, carboxylic acid or alkyl carboxylic acid and esters thereof, hydroxyl or alkyl hydroxyl.

Suitably, the couplable functional group may be represented by R* or (chain)-R*, wherein (chain)-R* is preferably (chain)_{b}-NH₂, (chain)_{b}-COOH, (chain)_{b}-OH. Suitably b is 0 or 1. When b is 0, the couplable functional group R* is —NH₂, —COOH or —OH directly attached to the 5- or 6-membered ring. When b is 1, (chain) is present. Suitably, b is 0, and (chain) is absent and R* is directly substituted onto X. Suitably, R* is NH₂ directly substituted onto X.

Preferably, when b is 1, (chain) is an alkyl, an alkenyl or an alkynyl functional group. Suitably, preferred (chain) is a C₁ to C₁₀ alkyl, C₂ to C₁₀ alkenyl or C₂ to C₁₀ alkynyl functional group. More suitably, (chain) is a C₁ to C₆ alkyl, more preferably a C₁ to C₃ alkyl; a C₂ to C₆ alkenyl, more preferably, a C₂ to C₃ alkenyl; or a C2 to C₆ alkynyl, more preferably, a C₂ or C₃ alkynyl group. Preferably, (chain) is a C₂ or a C₃ alkyl group. Suitably, (chain) may be branched or unbranched. Suitably, (chain) may be saturated or unsaturated. Where the chain is saturated it may comprises one or more double or triple bonds or combinations of same. Desirably, (chain) may be optionally substituted as described herein. Preferably, (chain) is (CH₂)ₙ, wherein n is 1, 2, 3, 4, 5, or 6. Preferably, n of (chain) is 1, 2, 3, or 4, most preferably n is 1, 2 or 3. Preferably, where n is 1, chain (CH₂)ₙ represents methyl. Desirably where n is 2, chain is (CH₂)ₙ represents ethyl. Preferably, where n is 3, chain (CH₂)ₙ represents butyl. A methyl, ethyl or propyl (chain) is particularly preferred.

In addition to the couplable functional group, the 5- or 6-membered ring of the precursor may be further substituted with one or more additional substituent groups as defined herein.

Further, the 5- or 6-membered ring may be fused to an aromatic carbocyclic, aromatic heterocyclic ring, aliphatic carbocyclic or aliphatic heterocyclic ring system^{RS} which itself may be optionally substituted. Suitable ring system^{RS} include the 5- and 6-membered rings described above. Additional fused rings include 2,3-dihydro-1H-indene, indene, indoline, 3H-indole, 1H-indole, 2H-isoindole, and indolizine.

It should be understood that the 5- or 6-membered ring when fused to a ring system^{RS} forms a core ring structure of the non-ionic hydrophilic head group precursor compound and the non-ionic head group of the anti-agglomerant compound of the invention.

The ring or ring system^{RS} may be substituted with one or more of hydrogen, halogen, hydroxyl, ether, alkyl hydroxyl, alkyl ether, oxo, ═S, carboxylic acid, carboxylic acid ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible. Optional substituents include one or more of H. C₁-C₆ alkyl, hydroxyl, amine, amide, COOH, alkyl ester or halogen. In some embodiments, no substituent is preferred, for example, in the case where ring^{a} is simple 5 membered ring, for example, a 5 membered heterocyclic ring, more especially a 5 membered heterocylic aliphatic ring such as pyrrolidine.

In some embodiments, an alkyl ester or an oxo substituent is particularly preferred. Alkyl ester substituents, particularly ethyl ester substituents are preferred, especially in the case where ring^{a} is a 6 membered ring, for example, a 6 membered heterocyclic or carbocyclic ring, more especially a 6 membered carboxcyclic aromatic ring such as phenyl. Oxo substituents are especially preferred in the case where ring^{a} is a 5 membered ring, for example, a 5 membered heterocyclic ring, more especially a 5 membered heterocylic aliphatic ring such as pyrrolidine.

The ring or ring system^{RS} may be substituted with one or more of halogen, H, OH, OR′, (CH₂)ₙOH, (CH₂)ₙOR′, ═O, C(O)OH, C(O)OR′, (CH₂)ₙC(O)OH, (CH₂)ₙ(O)OR′, NH₂, NHR′, NR′R′, (CH₂)ₙNH₂, (CH₂)ₙNHR′, (CH₂)ₙNR′R′, S(O)₂OH, wherein n is 1 to 20 and R′ is independently selected from the group consisting of H, alkyl, amino, hydroxyl, ether, carboxyl, acyl including ester and aldehyde, a 5- or 6-membered carbocyclic or heterocyclic ring which can be optionally substituted; and wherein KM is selected from the group of functionalities consisting of: alkyl, acyl, carbonyl, carboxyl, alkyl carboxyl, dicarboxyl for example oxalate, malonate, succinate, glutarate, adipate, etc., ester, alkyl ester, diester, dicarboxylate ester, amine, alkyl amino, amido, alkyl amido, ether, alkyl ether, guanidino, alkyl guanidino, imino, alkyl imino, sulfhydryl, sulfonyl, sulfinyl, sulfenyl, phosphoryl, diphosphoryl, thioester, alkyl thioester, each of which may be optionally substituted where structurally possible with one or more of C₁-C₆ alkyl, hydroxyl, carbonyl including acyl, carboxyl, halo, amino or amido functionalities.

The ring or ring system^{RS} may be substituted with one or more of a 5- or 6-membered carbocyclic or heterocyclic ring which itself may be optionally substituted. Such rings or ring system^{RS} may be selected from benzyl, phenyl, pyrrolidyl, pyrrolyl, pyrrolidonyl, pyridyl, imidazyl, morpholinyl, phenethyl, napthyl, nictinoyl, or piperonyl. A pyrrolyl ring is a particularly preferred substituent.

The ring or ring system^{RS} may be substituted with KM, wherein KM is selected from the group of functionalities consisting of: optionally substituted alkyl carboxylate, optionally substituted alkyl dicarboxylate, optionally substituted alkyl dicarboxylate ester, optionally substituted alkyl amide, optionally substituted alkyl ether, optionally substituted alkyl ester, carboxyl, dicarboxyl, dicarboxyl ester, amido, ester, amino, wherein the optional substituents are independently selected from one or more of C₁-C₆ alkyl, hydroxyl, carbonyl including acyl such as aldehyde and ketone, carboxyl, and amido, halo, or amino functionalities.

In a preferred embodiment, the non-ionic hydrophilic head group component is derived from a precursor compound having the following general formula:

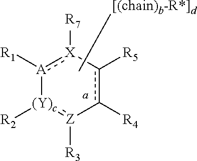

in which, d is 1, 2 or 3 and b is 0 or b is 1, provided that when b is 1, (chain) is an optionally substituted alkyl, an optionally substituted alkenyl or an optionally substituted alkynyl functional group;
   R* is —NH₂, —COOH or —OH, most preferably R* is NH₂;
   each of A, Y. Z of ring^{a} is independently CH, C, N, O, or S, and
   each instance of R¹, R², R³ is independently a lone pair or R′, provided that when A, Y and Z are independently O or S, the corresponding R¹, R² or R³ is a lone pair,
   X is CH, C, or N, and
wherein when c is 0. Y is absent, or when c is 1, Y is present, provided that ring is not a difatty ester substituted tetrahydrofuran ring, particularly not a distearate substituted fatty ester tetrahydrofuran ring;
each instance of R¹, R⁵, R⁶, and R⁷ when not L, is independently selected from hydrogen, halogen, hydroxyl, ether, alkyl hydroxyl, alkyl ether, oxo, thio, carboxylic acid, carboxylic acid ester, carboxylic acid alkyl ester alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfonic acid, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
and when not L, each of R¹ and R², R² and R³, R³ and R⁴, and R⁴ and R⁵, when taken together independently form a 5- or 6-membered optionally substituted aromatic carbocyclic, optionally substituted aromatic heterocyclic ring, optionally substituted aliphatic carbocyclic or optionally substituted aliphatic heterocyclic ring which can be optionally substituted with one or more substituents;
wherein optional substituents are independently selected from with one or more of H, C₁-C₆ alkyl, acyl, carboxylic acid, carboxylate ester, alkyl carboxylic acid, alkyl carboxylate ester, hydroxyl, alkyl hydroxyl, sulfonic acid, amine, amide or halogen.

Desirably, R* is —NH₂. Amine is preferred as it is readily couplable to the —COOH of fatty acids.

In a preferred embodiment, the non-ionic hydrophilic head group component is derived from a precursor compound having the following general formula:

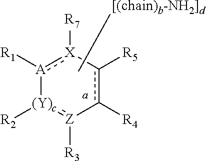

in which, d is 1, 2 or 3 and L represents
   b is 0 or b is 1, provided that when b is 1, (chain) is an optionally substituted alkyl, an optionally substituted alkenyl or an optionally substituted alkynyl functional group;
   each of A, Y, Z of ring^{a} is independently CH. C, N, O, or S, and
   each instance of R¹, R², R³ is independently a lone pair or R′, provided that when A, Y and Z are independently O or S, the corresponding R¹, R² or R³ is a lone pair,
   X is CH, C, or N, and
wherein when c is 0, Y is absent, or when c is 1, Y is present, provided that ring^{a} is not a difatty ester substituted tetrahydrofuran ring, particularly not a distearate substituted fatty ester tetrahydrofuran ring;
each instance of R⁴, R⁵, R⁶, and R⁷ when not L, is independently selected from hydrogen, halogen, hydroxyl, ether, alkyl hydroxyl, alkyl ether, oxo, thio, carboxylic acid, carboxylic acid ester, carboxylic acid alkyl ester alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfonic acid, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
and when not L, each of R¹ and R², R² and R³, R³ and R⁴, and R⁴ and R⁵, when taken together independently form a 5- or 6-membered optionally substituted aromatic carbocyclic, optionally substituted aromatic heterocyclic ring, optionally substituted aliphatic carbocyclic or optionally substituted aliphatic heterocyclic ring which can be optionally substituted with one or more substituents;
wherein optional substituents are independently selected from with one or more of H, C₁-C₆ alkyl, acyl, carboxylic acid, carboxylate ester, alkyl carboxylic acid, alkyl carboxylate ester, hydroxyl, alkyl hydroxyl, sulfonic acid, amine, amide or halogen.

It should be understood that the dashed line represents a chemical bond that may be a single bond or a multiple bond, for example, double or a triple bond, in accordance with conventional chemical valence and structure rules.

As used herein, CH represents a saturated tetravalent carbon (—CH—). C represents an unsaturated tetravalent carbon (C(—)₄), N represents a saturated trivalent N (—N═) or an unsaturated trivalent N (N(—)₃) in which case R¹, R² or R³ represents a lone pair, O represents a divalent O in which case R¹, R² or R³ represents a lone pair, and S represents a divalent S (—S—) in which case R¹, R² or R³ represents a lone pair.

Suitably, d is 1 or 2. Desirably, when d is 2, each (chain)_{b} can the same or different.

Preferably, the precursor compound has one of the following general formulas:

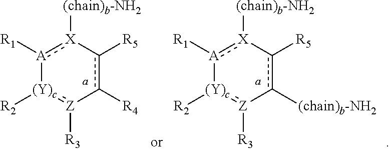

Suitably, when any of A, Y, Z is O, and the ring is an aliphatic carbocyclic ring, preferred anti-agglomerants comprise less than two, or more than two, of R¹, R², R³, R⁴, and R⁵ being simultaneously a fatty ester thereof.

Suitably, the alkyl hydroxyl is (CH₂)ₙOH, where n is from 1 to 10, n is from 1 to 10, preferably, 1 to 5, most preferably from 1 to 3.

Suitably, carboxylic acid alkyl ester is C(O)O(CH₂)ₙH, n is from 1 to 10, preferably, 1 to 5, most preferably from 1 to 3.

Suitably, alkyl carboxylic acid is (CH₂)ₙC(O)OH, wherein n is from 1 to 10, preferably, 1 to 5, most preferably from 1 to 3.

Suitably, each instance of R⁴, R⁵ and R⁶ may independently be halo, H, OH, OR′, (CH₂)ₙOH, (CH₂)ₙOR′, ═O, ═S, C(O)OH, C(O)OR′, (CH₂)ₙC(O)OH, (CH₂)ₙ(O)OR′, NH₂, NHR′, NR′R′, (CH₂)ₙNH₂, (CH₂)ₙNHR′, (CH₂)ₙNR′R′, S(O)₂OH, wherein n is 1 to 10 and R′ is independently selected from the group consisting of H, C₁-C₆ alkyl, hydroxyl, amine, amide or halogen, a 5- or 6-membered carbocyclic or heterocyclic ring which can be optionally substituted.

Preferably, n of R′ is 1 to 10, more preferably 1 to 6. Most preferred n is 1, 2, or 3 representing methyl, ethyl, propyl.

Preferred optional substituents are C₁-C₆ alkyl, hydroxyl, amine, amide or halogen. Most preferably, an optional substituent is H or C₁-C₆ alkyl.

Halogen is selected from is chloro, bromo, fluoro, or iodo, most preferably chloro or fluoro.

Preferably, the ring or ring system is an aromatic carbocyclic or an aromatic heterocyclic ring.

Suitably, c is 0 and the ring is a 5-membered ring.

Suitably, c is 1 and the ring is a 6-membered ring.

Preferably. X is selected from CH, C or N. In another embodiment, X is CH or N. Suitably, X is CH or C. Preferably, X is C.

Suitably, Z is selected from CH, C, N, O or S. Desirably, Z is CH or C. Preferably, Z is C.

Preferably the ring is a heterocyclic aromatic ring.

Suitably, when Z is O, N or S. R³ is a lone pair. In a preferred embodiment, Z is N or O.

Suitably, Z is N and R³ is a lone pair.

Desirably, each of Z and X is CH, C or N.

Suitably, A is selected from CH, C, N, O or S. Desirably, A is CH, C, or N. Preferably, A is CH or C. Most preferably, A is C. In desirable embodiments, A is N and X is C.

Preferably, a is 1 and Y is present. Suitably, Y is CH or C. Most preferably Y is C.

Preferably, both Y and 2 are C.

Suitably, X, A, and Y are CH or C.

Suitably, X, A, and Y form part of an aromatic ring.

Suitably, A and Z may independently be C or N. Preferably both A and Z are C.

Preferably, A, X and Z are C.

In embodiments where at least one of X, A, Y and Z is N, O or S, the ring is a heterocyclic ring.

Suitably, the heterocyclic ring is an aromatic heterocyclic ring.

Most preferably, each of X, A, Y and Z is CH or C forming a carbocyclic ring.

Suitably, the carbocyclic ring is an aromatic carbocyclic ring.

In some embodiments, the ring is aliphatic. In some embodiments, the ring is an aliphatic heterocyclic ring.

Preferably, the ring is di-substituted, tri-substituted, tetra-substituted or penta-substituted ring whereby the substitutions are at one or more of R¹-R⁵.

Preferably, the ring is a di-substituted carbocyclic or heterocyclic aromatic or aliphatic ring or a tri-substituted carbocyclic or heterocyclic aromatic or aliphatic ring, a tetra-substituted carbocyclic or heterocyclic aromatic or aliphatic ring, or penta-substituted carbocyclic or heterocyclic aromatic or aliphatic ring.

Most preferably, the ring is a carboxylic acid or ester substituted ring, a di-carboxylic acid or ester substituted ring or a tri-carboxylic acid or ester substituted ring, a tetra-carboxylic acid or ester substituted ring, or penta-carboxylic acid or ester substituted ring. Most preferably, the ring is a di-carboxylic acid substituted ring particularly an aromatic carbocyclic ring.

Suitably, b is 0, and (chain) is absent. When (chain) is absent. NH₂ is directly substituted onto X. Suitably, b is 0. (chain) is absent and each of R¹, R⁴ and R⁵ is H.

Suitably, the compound comprises a substituted carbocyclic or heterocyclic aromatic or aliphatic ring and has one of the following general structures:

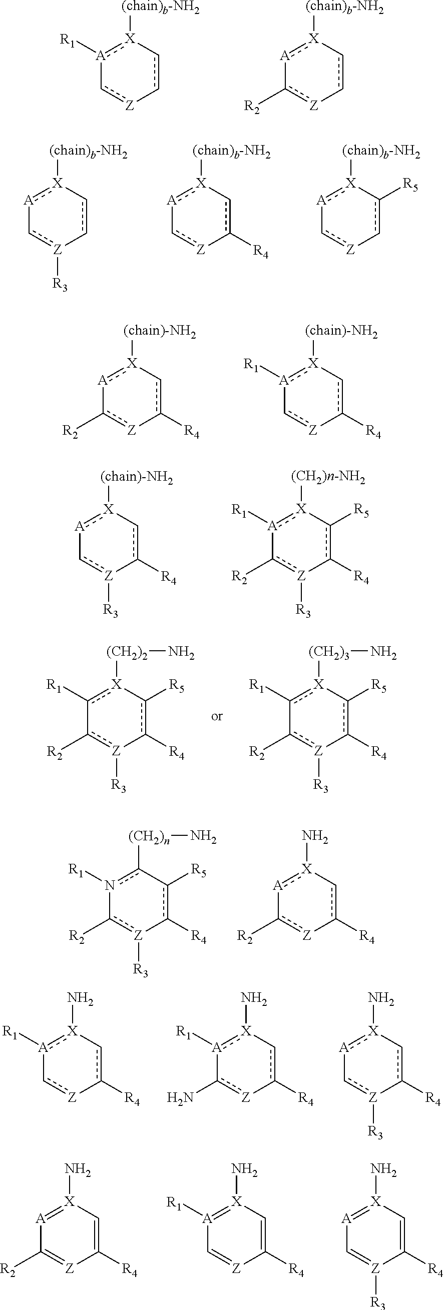

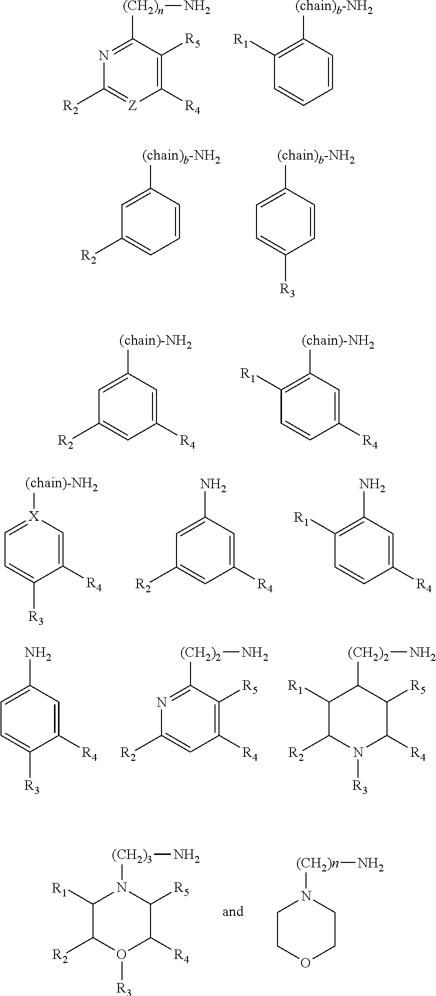

Where R¹ and R², R² and R³, R³ and R⁴, and R⁴ and R⁵ independently form a 5- or 6-membered aromatic or aliphatic carbocyclic or heterocyclic ring, the formed ring is preferably fused to the ring^{a} forming a ring system^{RS}. The 5- or 6-membered ring when fused to a ring system^{RS} as described form a core ring structure of the non-ionic hydrophilic head group. Suitably, the fused ring may be optionally substituted in accordance with conventional valence and chemical structure rules. Generic examples of head groups comprising simple fused ring systems include:

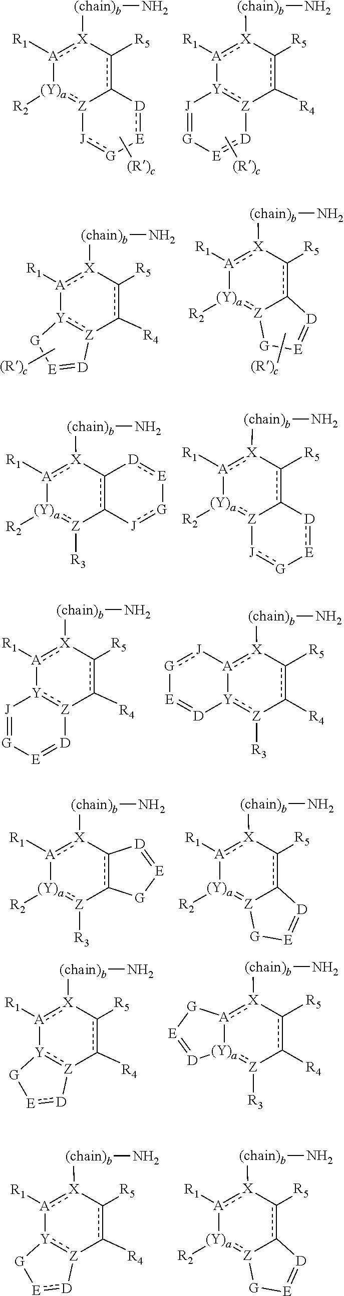

wherein each of D, E, G, and J are independently CH, C, N, O, or S. Suitably, each of D, E, G, and J is independently CH, C, or N, preferably, independently CH or C. Desirably, each of D and G is independently CH, C or O. Suitably, E is CH. Desirably, c is 0 to 3. Suitably, where c is 0, R′ is absent. Preferably c is 0 or 1. R′ is described above.

In one embodiment, the non-ionic hydrophilic head group component of the anti-agglomerant of the invention is derived from a compound having one of the following general formulas:

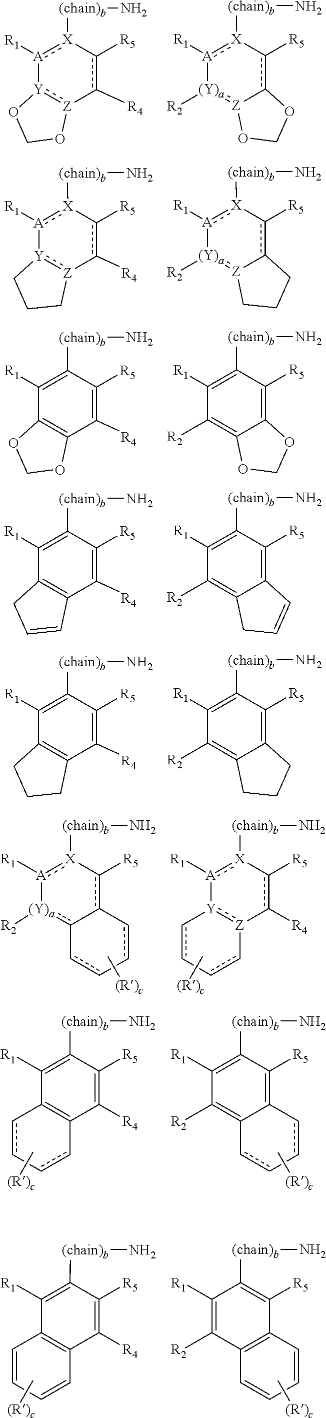

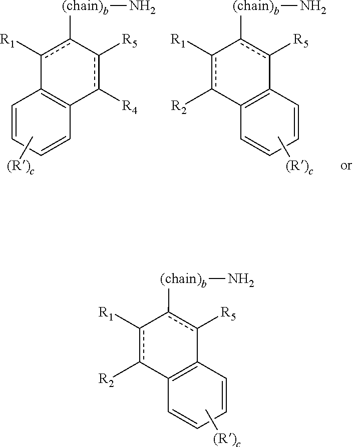

Specific examples of core ring structures include indene, dihydroindene, indoline, 3H-indole, 1H-indole, 2H-isoindole, indolizine, 1H-indazole, benzimidazole, 7-azaindole, 4-azaindole, 5-azaindole, 6-azaindole, 7-azaindazole, pyrazolo[1,5-a]pyrimidine, purine, benzofuran, isobenzofuran, benzofuran, benzo[c]thiophene, benzo[b]thiophene, 1,2-benzisothiazole-3(2H)-one, adenine, guanine, decahydrolsoquinoline, decahydriquinoline, tetrahydroquinoline, 1,2-dihydroquinoline, 1,2-dihydroisoquinoline, quinoline, isoquinoline, 4H-quinoline, quinoxaline, phthalazine, quinazoline, cinnoline, 1,8-naphthyridine, pyrido[3,2-d]pyrimidine, pyrido[4,3-d]pyrimidine, pyrido[3,4-b]pyrazine, pyrido[2,3-b]pyrazine, pteridine, 2H-chromene, 1H-isochromene, 3H-isochromene, 2H-chromen-2-one, 2H-benzo[e][1,2]oxazine, 2H-benzo[e][1,2]oxazine, 2H-benzo[e][1,3]oxazine, 2H-benzo[b][1,4]oxazine, quinolin-2(1H)-one, isoquinolin-2(1H)-one.

Most preferably, the compound has a napthyl core structure. Preferably, c is 1 and the headgroup has one of the following structures:

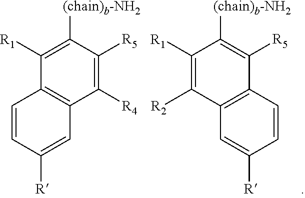

Suitably, R′ is selected from OH, OR′, NH₂, COOH, or COOR′. Most preferably, R′ is COOH.

Suitably, the compound comprises a substituted carbocyclic or heterocyclic aromatic or aliphatic ring and has one of the following general structures:

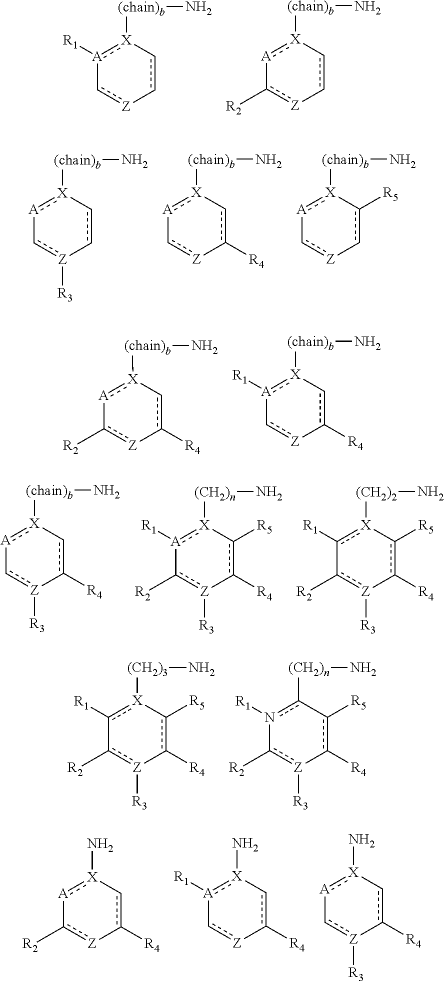

Aromatic ring analogues include the following general structures:

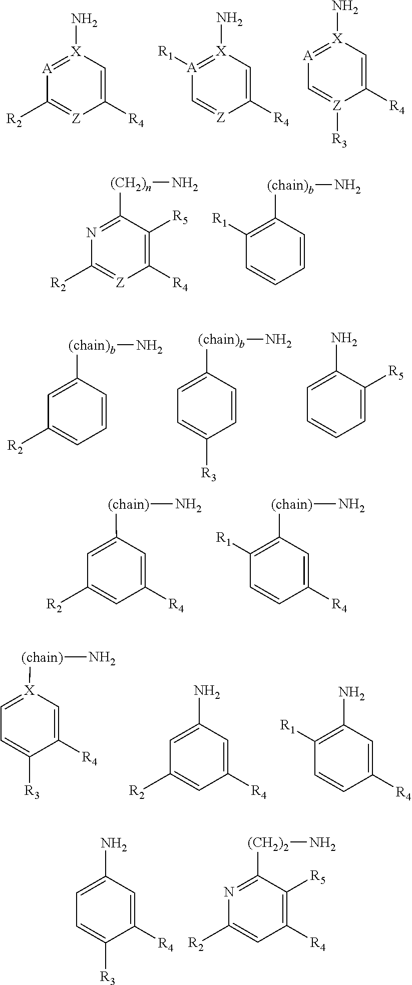

Suitably, the ring is an aromatic carbocyclic ring having one of the following general structures:

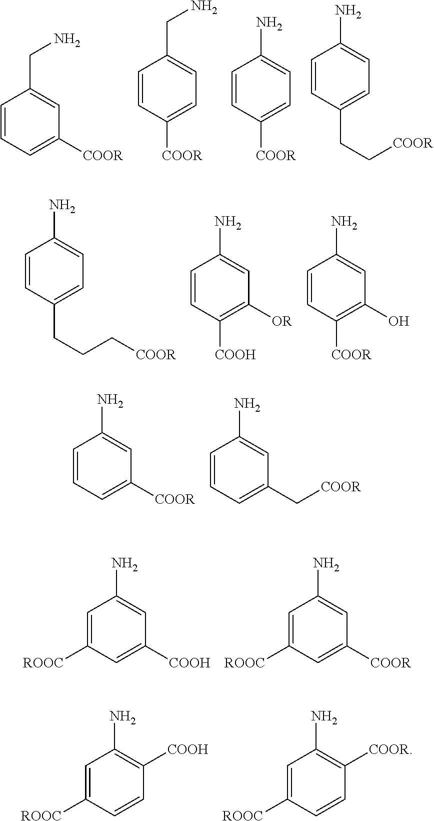

Suitably, R is H or alkyl.

Suitable aliphatic rings include the following general structures:

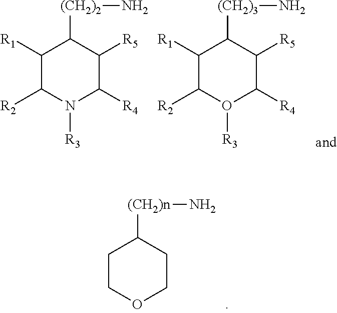

In another embodiment still, c is 0, and the compound from which the non-ionic hydrophilic head group component of the anti-agglomerant of the Invention is derived has one of the following general structures:

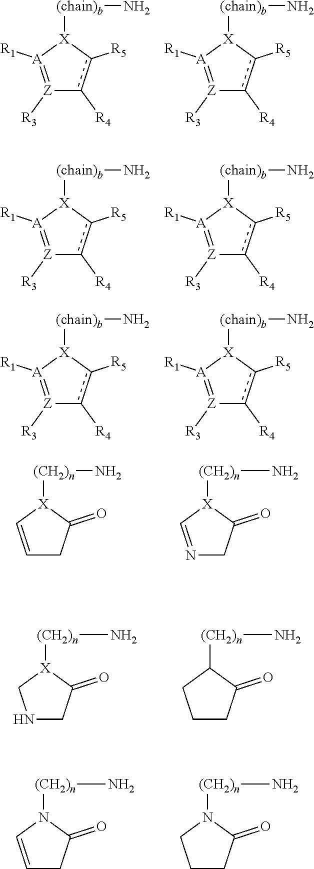

An exemplary compound from which the non-ionic hydrophilic head group component of the anti-agglomerant of the invention is derived is selected from the group consisting of:

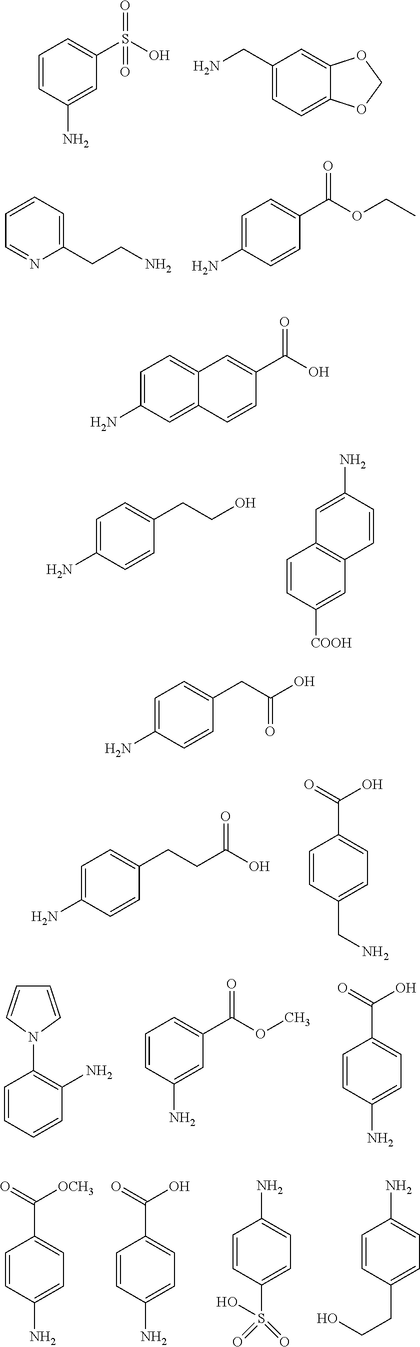

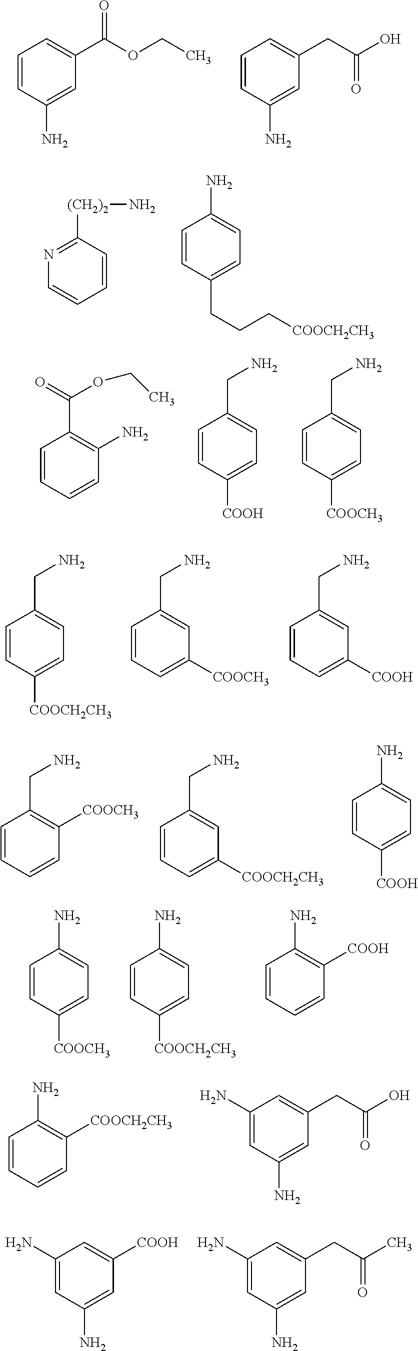

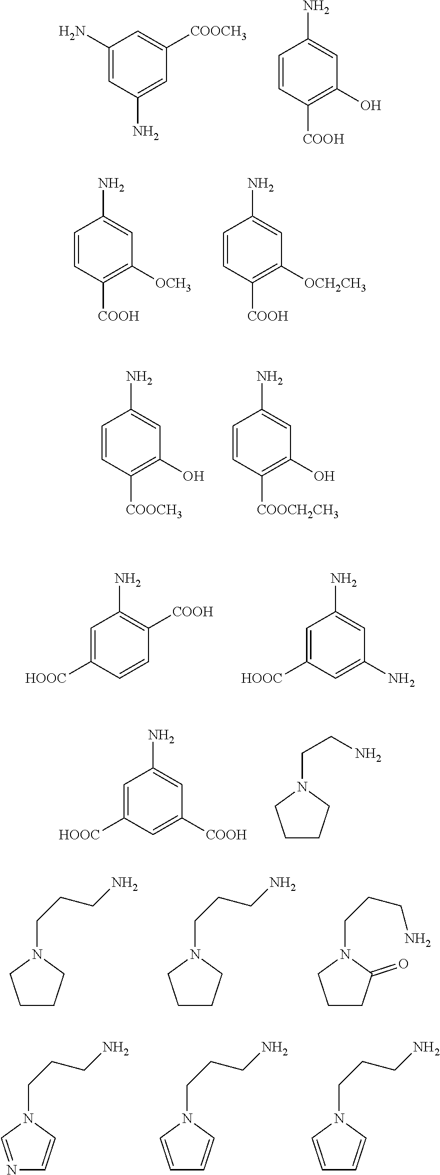

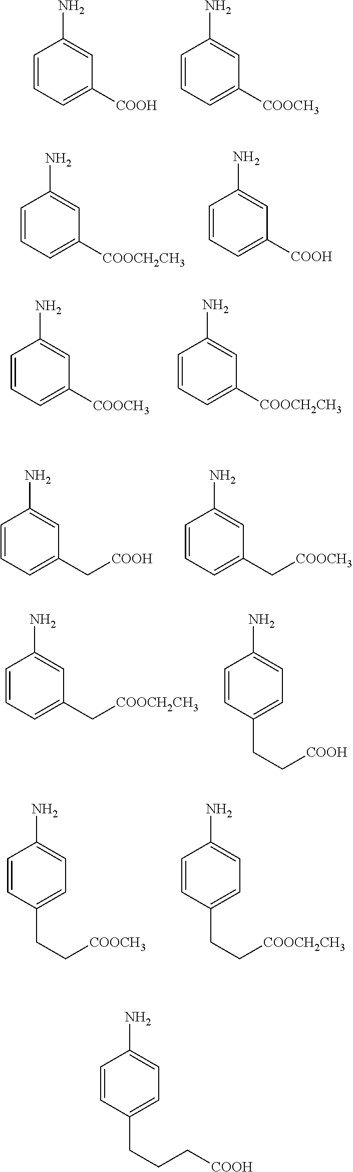

### Preferred Anti-Agglomerant Molecules

In one aspect, there is provided an oil soluble anti-agglomerant molecule comprising

at least one non-ionic hydrophilic head group component and at least one lipophilic tail component, wherein the non-ionic hydrophilic head group component of the anti-agglomerant of the invention is derived from a precursor compound comprising:

a 5- or 6-membered carbocyclic or heterocyclic ring which is not a difatty ester tetrahydrofuran ring, and which is substituted with at least one functional group couplable to at least one lipophilic tail group.

In one embodiment, the 5- or 6-membered carbocyclic or heterocyclic ring is not a trifatty ester substituted tetrahydrofuran ring. In one embodiment, the 5- or 6-membered carbocyclic or heterocyclic ring is not a mono fatty ester substituted tetrahydrofuran ring. In another embodiment, the 5- or 6-membered carbocyclic or heterocyclic ring is not a dihydroxysubstituted tetrahydrofuran ring. In another embodiment, the 5- or 6-membered carbocyclic or heterocyclic ring is not a hydroxysubstituted tetrahydrofuran ring and/or fatty ester derivative thereof. In another embodiment, the 5- or 6-membered carbocyclic or heterocyclic ring is not a tetrahydrofuran polyol ring. In embodiments where the ring is a dihydroxyl substituted tetrahydrofuran ring, and where the compound comprises three lipophilic tail components, for example, one or more stearate chains, it is preferred that each tail component is linked to the ring by a functional group not being an ester functionality, for example, in such embodiments, an amide linkage is preferred. In particular, sorbitan stearates, particularly, sorbitan monostearate, sorbitan distearate, and sorbitan tristearate, are excluded from the anti-agglomerants of the invention. Suitably, the anti-agglomerant is not an alkyl polyglucoside or polysorbates, including a sorbitan fatty acid ester based surfactant such as sorbitan tristearate or sorbitan monooleoate. Additional excluded rings for some embodiments are discussed in detail above.

Suitably, the compound comprises one hydrophilic head group as described.

Suitably, one or more of the non-ionic hydrophilic head group component and/or the lipophilic tail component of the anti-agglomerant may be derived from a water soluble naturally occurring product, a precursor to, a metabolite or a derivative thereof, as described herein.

Preferably, linker group L, (chain)_{b}-R*, wherein b is 0 or 1 and R* is an amide (NHCO or CONH), an imine (C═N or N═C), an ester (C(O)O or OC(O)), ether or thioester (C(O)S or SC(O)) functionality, links the at least one non-ionic hydrophilic head group component and the at least one lipophilic tail component.

Suitably, from 1 to 3 linker groups L may be present. However, compounds having 1 and 2 linker groups are particularly preferred. Suitably, from 1 to 3 linker groups L bonded to the lipophilic tail may be present. However, compounds having 1 and 2 linker groups bonded to lipophilic tails are particularly preferred. Suitable, lipophilic tails are described herein.

In a preferred aspect of the invention, the anti-agglomerant compound comprises:

at least one non-ionic hydrophilic head group component and at least one lipophilic tail component coupled by at least one linker moiety L, wherein L comprises:
(i) a group (chain)_{b}-R*, wherein R* is an amide (NHCO or CONH), an imine (C═N or N═C), an ester (C(O)O or OC(O)), ether or thioester (C(O)S or SC(O)) linkage between the at least one non-ionic hydrophilic head group component and the at least one lipophilic tail component, wherein b is 0 or 1, and (chain) is an optionally substituted alkyl, an optionally substituted alkenyl or an optionally substituted alkynyl functional group, or
(ii) ring system^{RS} which is a carbocyclic or heterocyclic ring system^{RS} linking the at least one non-ionic hydrophilic head group component and the at least one lipophilic tail component; and

wherein the at least one non-ionic hydrophilic head group component comprises an optionally substituted 5- or 6-membered carbocyclic or heterocyclic ring which is not a difatty ester substituted tetrahydrofuran ring, particularly not a distearate substituted fatty ester tetrahydrofuran ring, and

wherein at least one of the lipophilic tail components comprises at least one fatty chain.

Preferably, the ring system^{RS} has 2 to 10 fused 4, 5 or 6-membered rings, more preferably, 2, 3, 4, 5 or 6 fused 4-, 5- or 6-membered rings, most preferably, 1, 2 or 3 fused 6-membered rings.

In other embodiments, an optionally substituted 5- or 6-membered carbocyclic or heterocyclic ring is not a fatty ester derivative of a tetrahydrofuran polyol ring.

Suitably, the optional substituents for (chain) of the linker L, included one or more substituents which are independently selected from H, C₁-C₆ alkyl, acetyl, carboxylic acid, carboxylate ester, alkyl carboxylic acid, alkyl carboxylate ester, hydroxyl, alkyl hydroxyl, sulfonic acid, amine, amide or halogen.

Preferably, the at least one non-ionic hydrophilic head group component is derived a precursor compound as described elsewhere herein.

Desirably, the at least one at least one lipophilic tail component is derived from and/or comprises a fatty chain. Preferred fatty chains are represented by fatty acids including C₄-C₂₀ hydrocarbon chains, more preferably, C₆-C₁₆ hydrocarbon chains alkyl carboxylic acids. Particularly preferred are C₆-C₁₂ hydrocarbon chains. C₈, C₉, C₁₀, C₁₁, C₁₂, C₁₃ and C₁₆ fatty chains are further preferred. C₈, C₁₀, C₁₃ fatty chains are particularly preferred. Suitably, the fatty chains may be branched or unbranched. Suitably, the fatty chains may be saturated or unsaturated. Most preferably, fatty chains are derived from a fatty acid, a fatty amine, a fatty ester, a fatty aldehyde, a fatty ether, a fatty nitriles, or a fatty alcohol, preferably, n-octaneoic acid, n-decanoic acid and n-tridecanoic acid. Particularly preferred fatty chains are n-octane, n-decane and n-tridecane alkyl chains. Particularly preferred fatty chains include those derived from n-octaneoic acid, n-decanoic acid and n-tridecanoic acid. Preferably, the fatty chain is a decane chain.

A preferred anti-agglomerant has the following general formula:

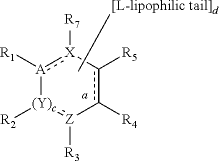

in which d is 1, 2 or 3, and
L represents
(i) (chain)_{b}-R*, wherein R* is an amide (NHCO or CONH), an imine (C═N or N═C), an ester (C(O)O or OC(O)), ether or thioester (C(O)S or SC(O)) linkage between ring^{a} and the lipophilic tail component at any one of R¹, R², R¹, R⁴, R⁵, and R⁷ of ring^{a}, wherein b is 0 or 1, and (chain) is an optionally substituted alkyl, an optionally substituted alkenyl or an optionally substituted alkynyl functional group, or
(ii) a ring system^{RS} attached to ring^{a} as a substituent at any one of R¹, R², R³, R¹, R⁵, and R⁷, and linking ring^{a} to the lipophilic tail, or a ring system^{RS} directly fused to ring^{a} at any adjacent pair of R¹ and R², R² and R³, R³ and R⁴, R⁴ and R⁵ or R⁵ and R⁶,
   wherein the ring system^{RS} is an aromatic or aliphatic carbocyclic or heterocyclic ring system which may be saturated or unsaturated and which may be optionally substituted; and wherein
each of A, Y, Z of ring^{a} is independently CH, C, N, O, or S, and
   each instance of R¹, R², R³ is independently a lone pair or R⁶, provided that when A, Y and Z are independently O or S, the corresponding R¹, R² or R³ is a lone pair,
   X is CH, C, or N, and
wherein when c is 0, Y is absent, or when c is 1, Y is present, provided that ring^{a} is not a difatty ester substituted tetrahydrofuran ring, particularly not a distearate substituted fatty ester tetrahydrofuran ring;
each instance of R¹, R⁵, R⁶, and R⁷ when not L, is independently selected from hydrogen, halogen, hydroxyl, ether, alkyl hydroxyl, alkyl ether, oxo, thio, carboxylic acid, carboxylic acid ester, carboxylic acid alkyl ester alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfonic acid, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
when not L, each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted;
wherein the optional substituents are independently selected from with one or more of H, C₁-C₆ alkyl, acyl, carboxylic acid, carboxylate ester, alkyl carboxylic acid, alkyl carboxylate ester, hydroxyl, alkyl hydroxyl, sulfonic acid, amine, amide or halogen; and
wherein the at least one lipophilic tail component comprises at least one fatty chain.

Suitably, when any of A, Y, Z is O, and the ring is an aliphatic carbocyclic ring, preferred anti-agglomerants comprise less than or more than two of R¹, R², R³, R⁴, and R⁵ being simultaneously OH or fatty ester thereof.

Suitably, the alkyl hydroxyl is (CH₂)ₙOH, where n is from 1 to 10, n is from 1 to 10, preferably, 1 to 5, most preferably from 1 to 3.

Suitably, carboxylic acid alkyl ester is C(O)O(CH₂)ₙH, n is from 1 to 10, preferably, 1 to 5, most preferably from 1 to 3.

Suitably, alkyl carboxylic acid is (CH₂)ₙC(O)OH, wherein n is from 1 to 10, preferably, 1 to 5, most preferably from 1 to 3.

Preferred compounds have the following general structure:

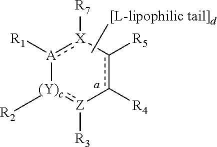

in which d is 1, 2 or 3 and L represents
(i) (chain)_{b}-R*, wherein R* is an amide (NHCO or CONH), an imine (C═N or N═C), an ester (C(O)O or OC(O)), ether or thioester (C(O)S or SC(O)) linkage between ring^{a} and the lipophilic tail component at any one of R¹, R², R³, R⁴, R⁵, and R⁷ of ring^{a}, wherein b is 0 or 1, and (chain) is an optionally substituted alkyl, an optionally substituted alkenyl or an optionally substituted alkynyl functional group, or
(ii) a ring system^{RS} attached to ring^{a} as a substituent at any one of R¹, R², R³, R⁴, R⁵, and R⁷, and linking ring^{a} to the lipophilic tail, or a ring system^{RS} directly fused to ring^{a} at any adjacent pair of R¹ and R², R² and R³, R³ and R⁴, R⁴ and R⁵, or R⁵ and R⁶, wherein the ring system^{RS} is an aromatic or aliphatic carbocyclic or heterocyclic ring system which may be saturated or unsaturated and which may be optionally substituted;
wherein when c is 0, Y is absent, or when c is 1, Y is present, provided that ring^{a} is not a difatty ester substituted tetrahydrofuran ring, particularly not a distearate substituted fatty ester tetrahydrofuran ring, and
   each of A, Y. Z of ring^{a} is independently CH. C, N, O, or S, and each instance of R¹, R², R³ is independently a lone pair or R⁶, provided that when A, Y and Z are independently 0 or S, the corresponding R¹, R² or R is a lone pair.
   X is CH, C, or N, and
each instance of R⁴, R⁵, R⁶, and R⁷ when not L, is independently selected from KM, halogen, H, OH, OR′, (CH₂)ₙOH, (CH₂)ₙOR′, ═O, ═S, C(O)OH, C(O)OR′, (CH₂)ₙC(O)OH, (CH₂)ₙ(O)OR′, C(O)O(CH₂)ₙR′, NH₂, NHR′, NR′R′, (CH₂)ₙNH₂, (CH₂)ₙNHR′, (CH₂)ₙNR′R′, S(O)OH, S(O)₂OR′, (CH₂)ₙS(O)₂OH, (CH₂)ₙS(O)₂OR′, NHS(O)₂OH, NHS(O)₂OR′, (CH₂)ₙNHS(O)₂OH, (CH₂)ₙNHS(O)₂OR′, NH(CH₂)ₙS(O)₂OH, NH(CH₂)ₙS(O)₂OH wherein n is 1 to 20 and R′ is independently selected from the group consisting of H, alkyl, amino, hydroxyl, ether, carboxyl, acyl including ester and aldehyde, a 5- or 6-membered carbocyclic or heterocyclic ring which can be optionally substituted; and wherein KM is selected from the group of functionalities consisting of: alkyl, acyl, carbonyl, carboxyl, alkyl carboxyl, dicarboxyl for example oxalate, malonate, succinate, glutarate, adipate, ester, alkyl ester, diester, dicarboxylate ester, amino, alkyl amino, amido, alkyl amido, ether, alkyl ether, guanidino, alkyl guanidino, imino, alkyl imino, sulfhydryl, sulfonic acid, sulfonyl, sulfinyl, sulfenyl, phosphoryl, diphosphoryl, thioester, alkyl thioester; or
when not L, each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted;
wherein the optional substituents are independently selected from one or more H, C₁-C₆ alkyl, acyl, carboxylic acid, carboxylate ester, alkyl carboxylic acid, alkyl carboxylate ester, hydroxyl, alkyl hydroxyl, sulfonic acid, amine, amide or halogen; and
wherein at least one lipophilic tail component comprises at least one fatty chain.

Preferred compounds have one of the following general formulas:

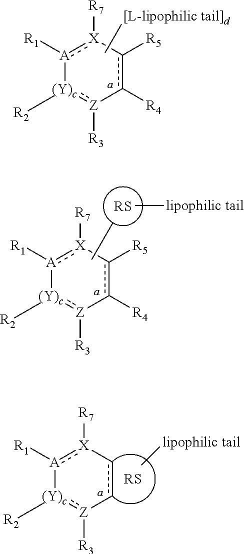

Preferably, the fused ring system^{RS} has 2 to 10 fused 4, 5 or 6-membered rings, more preferably, 2, 3, 4, 5 or 6 fused 4-, 5- or 6-membered rings, most preferably, 1, 2 or 3 fused 6-membered rings.

Most preferably, the lipophilic tail component is derived from and/or comprises a fatty chain such that particularly preferred compounds have one of the following general structures:

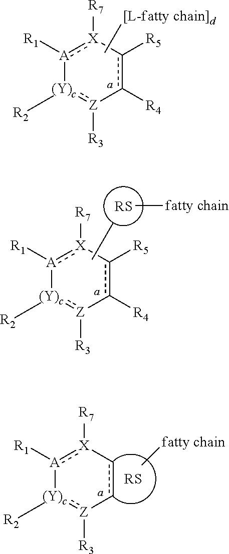

Preferably, d is 1 or 2, most preferably d is 1.

Preferably, the ring or ring system is optionally substituted with at least one group R′ as described elsewhere herein.

Suitably, R* or (chain)_{b}-R* is at R⁴ and/or R⁷ of ring^{a}. Suitably L is substituted on ring^{a} at R⁷. In this case d may be 1.

Desirably, b is 0 or 1.

In another embodiment, L is a group (chain)_{b}-R* wherein R* represents a carboxylate, an amide, an imine, or an ester linkage to the lipophilic tail. Preferably, R* is NHC(O), C(O)O, O, C(O)S or N═CH.

In another embodiment d is 2. Suitably, R* or (chain)_{b}-R* is at R⁴ and/or R⁷ of ring^{a}. In one embodiment, R* is at both R⁴ and R⁷. For example, the compound may have one of the following structures:

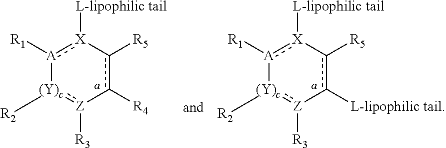

Particularly preferred compounds have one of the following general structures:

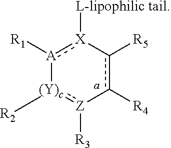

Desirably, L is (chain)_{b}-R* wherein, b is 0 or 1 and R* is a functionality selected from the group consisting of: an amide (NHCO or CONH), an imine (C═N or N═C), an ester (C(O)O or OC(O)), ether or thioester (C(O)S or SC(O)) linkage to the lipophilic tail component, provided that when b is 1, (chain) is an optionally substituted alkyl, an optionally substituted alkenyl or an optionally substituted alkynyl functional group;
wherein when c is 0, Y is absent, or when c is 1, Y is present, provided that ring^{a} is not a difatty ester substituted tetrahydrofuran ring, more particularly not a distearate substituted fatty ester tetrahydrofuran ring, and
   each of A, Y, Z of ring^{a} is independently CH, C, N, O, or S, and each instance of R¹, R², R³ is independently a lone pair or R⁶, provided that when A, Y and Z are Independently O or S, the corresponding R¹, R² or R³ is a lone pair,
X is CH, C, or N, and
each instance of R⁴, R⁵, R⁶, and R⁷ when not L, is independently selected from hydrogen, halogen, hydroxyl, ether, alkyl hydroxyl, alkyl ether, oxo, thio, carboxylic acid, carboxylic acid ester, carboxylic acid alkyl ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfonic acid, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
and when not L, each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted;
wherein the optional substituents are independently selected from with one or more of H, C₁-C₆ alkyl, acyl, carboxylic acid, carboxylate ester, alkyl carboxylic acid, alkyl carboxylate ester, hydroxyl, alkyl hydroxyl, sulfonic acid, amine, amide or halogen; and
wherein the at least one lipophilic tail component comprises at least one fatty chain.

In some embodiments, the ring is not a fatty ester derivative of a tetrahydrofuran polyol. However, a fatty amide derivative, a fatty thioester derivative, a fatty ether, or a fatty imide derivative of a tetrahydrofuran polyol ring is included in the scope of the invention.

Suitably, L is R′, is an amide, imine, or ester linkage between the non-ionic hydrophilic head group to the fatty chain. In another embodiment, L is a group (chain)_{b}-R* wherein R* represents a carboxylate, an amide, an imine, or an ester linkage to the fatty chain. Preferably, L is R* and R* is NHC(O), C(O)O, O, C(O)S, or N═CH.

Preferred anti-agglomerant have one of the following general formulas:

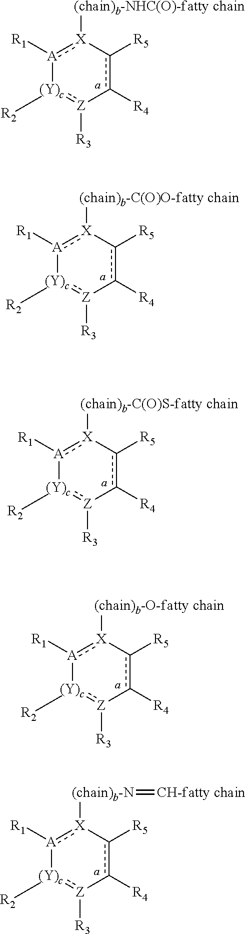

Suitably, ring^{a} is substituted at one of R⁴, R⁵, R⁶, and R⁷ as described above. Depending on the substituents, ring^{a} may comprise a phenyl, a benzoic acid or alkyl esters thereof, particularly a benzoic acid ester such as methylbenzoate, ethyl benzoate, a hydroxylbenzoic acid or alkyl esters thereof, an alkylbenzoic acid such as phenylacetic acid, phenylethanoic acid, phenylpropanoic acid, a phenylbutanoic acid, or alkyl esters thereof including phenylmethanoate, phenylethanoate, phenylpropanoate, a phenylbutanoate; an aromatic dicarboxylic acid such as phthalic acid, terephthalic acid, isophthalic acid, or an aromatic dicarboxylic acid alkyl ester thereof such as an isophthalic acid alkyl ester for example methylphthalate, dimethylterephthalate, or dimethylisophthalate; morpholinyl, tetrahydrofuranyl, oxopyrrolidnyl, pyrrolidnyl, piperidinyl, turanyl, imidazoyl, pyridinyl, pyrrolyl, benzodioxole, naphthyl, naphthoic acid, pyrrolylphenyl, or phenylsulfonic acid or esters thereof. In some embodiments, ring^{a} is preferably, ethyl benzoate ring, a pyrrolidnyl ring, or an oxopyrrolidnyl ring, particularly a 2-oxopyrrolidnyl ring. Anti-agglomerants having ring^{a} is preferably, ethyl benzoate ring, a pyrrolidnyl ring, or an oxopyrrolidnyl ring, particularly a 2-oxopyrrolidnyl ring, preferably further comprise C₈, C₁₀ or C₁₃ lipophilic chains (e.g., inclusive of C═O of amide linker group). In these embodiments, preferably b is 1 and (chain) is a C₂ or C₃ chain. Most preferably, L is R* is an amide linker directly attached to ring^{a}. Suitably, L is R* and R* is NHC(O). Alternatively, L is (chain)-R* wherein R* is amide and chain is methyl, ethyl or propyl. Particularly preferred anti-agglomerants include:

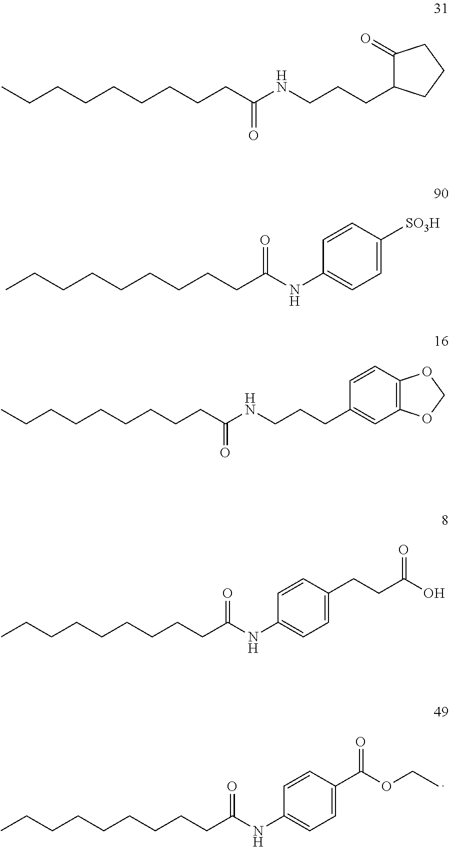 [SMILES: CCCCCCCCCC(=O)NCCCC1CCCC1=O ; CCCCCCCCCC(=O)Nc1ccc(S(=O)(=O)O)cc1 ; CCCCCCCCCC(=O)NCCCc1ccc2c(c1)OCO2 ; CCCCCCCCCC(=O)Nc1ccc(CCC(=O)O)cc1 ; CCCCCCCCCC(=O)Nc1ccc(C(=O)OCC)cc1]

Other preferred anti-agglomerants include:

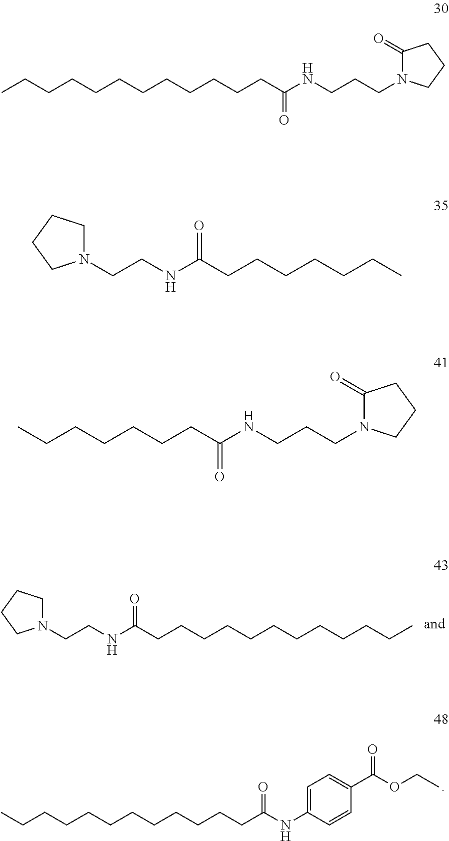

In a preferred embodiment, the anti-agglomerant compound comprises:

at least one non-ionic hydrophilic head group component and at least one lipophilic tail component coupled by at least one linker moiety L, wherein L comprises:

(i) a group (chain)_{b}-R*, wherein R* is an amide (NHCO or CONH), an imine (C═N or N═C), an ester (C(O)O or OC(O)), ether or thioester (C(O)S or SC(O)) linkage between the at least one non-ionic hydrophilic head group component and the at least one lipophilic tail component, wherein b is 0 or 1, and (chain) is an optionally substituted alkyl, an optionally substituted alkenyl or an optionally substituted alkynyl functional group. The groups and optionally substituents have been defined above.

In particularly, L, when (chain)-R* may be an optionally substituted alkyl, an optionally substituted alkenyl or an optionally substituted alkynyl functional group. Where (chain)-R* is substituted, it may be substituted with OH, COOH, COO(CH₂)ₙH, wherein n is 1-6. Suitably, (chain)-R* is (CH₂CHR^{\hat{ }})—R*, for example, (CH₂CHR^{\hat{ }})NHCO, wherein R^{\hat{ }} is the optional substituent, particularly —COOH. An example of such an embodiment is compound 3, which is based on phenylalanine. Other compounds having (chain)-R* as (CH₂CH(COOH))NHCO include compounds based on tyrosine (ring^{a} is a hydroxyphenyl ring) or based on tryptophan (ring^{a} is an indole ring). In some embodiments, (chain)-R* is not (CH₂CH(OH))—OC(O), particularly where the ring^{a} is a difatty ester derivative of tetrahydrofuran. However (chain)-R* as (CH₂CH(OH))—OC(O) may be allowed when the ring^{a} is a fatty amide derivative, a fatty thioester derivative, a fatty ether, or a fatty imide derivative of a tetrahydrofuran polyol ring is included in the scope of the invention.

In one embodiment, L represents a ring system^{RS} substituted onto to ring^{a} at any one of R¹, R², R³, R⁴, R⁵, and R⁷ and linking ring^{a} to the fatty chain in accordance with the following general structure:

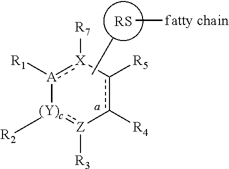

wherein when c is 0, Y is absent, or when c is 1, Y is present, provided that ring^{a} is not a difatty ester substituted tetrahydrofuran ring, and

each of A, Y, Z of ring^{a} is independently CH, C, N, O, or S, and each instance of R¹, R², R³ is independently a lone pair or R⁶; provided that when A, Y and Z are independently O or S, the corresponding R¹, R² or R³ is a lone pair,

X is CH, C, or N, and

each instance of R⁴, R⁵, R⁶, and R⁷ when not L, is independently selected from hydrogen, halogen, hydroxyl, ether, alkyl hydroxyl, alkyl ether, oxo, carboxylic acid, carboxylic acid ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or

and when not L, each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted; and

wherein the optional substituents are independently selected from with one or more of H, C₁-C₆ alkyl, hydroxyl, amine, amide or halogen.

In another embodiment, L is a ring system^{RS} which is an aromatic or aliphatic carbocyclic ring system^{RS} or an aromatic or aliphatic heterocyclic ring system^{RS} which can be saturated or unsaturated and may be unsubstituted or substituted with at least one group R′. Preferably, the ring system^{RS} is a 5-membered carbocyclic or a 5-membered heterocyclic ring, which may be unsaturated or saturated and may be unsubstituted or substituted with one or more R′. Preferably, the ring system^{RS} is a 6-membered carbocyclic or a 6-membered heterocyclic ring, which may be unsaturated or saturated and may be unsubstituted or substituted with one or more R′. Suitable, 5-membered carbocyclic rings include cyclopentane, and cyclopentadiene. Suitable, 5-membered heterocyclic rings include pyrrole, dioxolane, furan, tetrahydrofuran, thiophene, thiazole, imidazole subject to the above exclusions, Suitable, 6-membered carbocyclic rings include cyclohexane, cyclohexadiene. Suitable, 6-membered heterocyclic rings include pyridine, morpholine, pyran, tetrahydropuran, piperidine, pyrimidine, dioxane subject to the above exclusions.

A preferred ring system includes a fused ring system. The fused ring system may comprise a spiro fused ring in which the rings are joined at the same carbon. Alternatively, the fused ring system may comprise two ring junctions (bridgeheads) on adjacent carbons of a pair of carbons to which the ring are fused. In another embodiment, L is a ring system^{RS} directly fused to ring^{a} at any adjacent pair of R¹ and R², R² and R³, R³ and R⁴, R⁴ and R⁵; or R⁵ and R⁶.

Compounds having a ring system^{RS} directly fused to ring^{a} at R⁴ and R⁵ are particularly preferred in accordance with the following general structure:

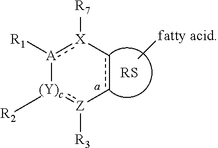

Preferably, the anti-agglomerant molecule, and preferably the lipophilic tail group of the anti-agglomerant molecule, preferably comprises a fused ring system^{RS}. Preferably, the fused ring system^{RS} has 2 to 10 fused 4, 5 or 6-membered rings, more preferably, the fused system has 2, 3, 4, 5 or 6 fused 4-, 5- or 6-membered rings, most preferably, 1, 2 or 3 fused 6-membered rings. In one embodiment, the ring system^{RS} comprises a fused ring system comprising from 1 to 5 rings. Preferably, the fused ring system comprises 1, 2 or 3 fused rings. Preferred fused ring systems comprise 2 fused 6-membered rings.

One or more of the rings or fused rings may be saturated or unsaturated rings. Where one or more of the fused rings is an unsaturated ring, that ring may independently be aromatic or non-aromatic. One or more of the fused rings may independently comprise one or more carbon, nitrogen, sulfur and/or oxygen atoms in accordance with conventional structure and valency rules.

The ring system^{RS} component of the anti-agglomerant may be selected from a dioxolane, cyclohexadiene, indene, 3H-indole, azulene, naphthalene, 1,2 dihydronaphthalene, and biphenylene. Such anti-agglomerants compounds therefore comprise core structures including:

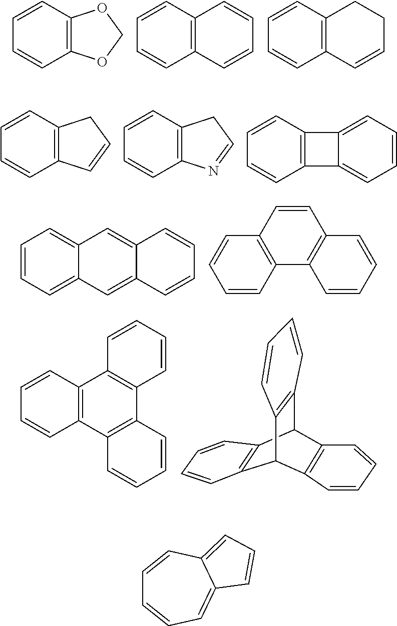

Dioxolane, and naphthalene rings are preferred. A particularly preferred fused ring arrangement provides a compound having chromane core structure:

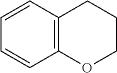

Other preferred fused ring systems provide a compound having steroid core structure:

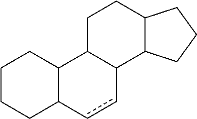

wherein the dashed line represent a single or double bond.

Each of the rings of the ring system^{RS} may independently substituted with optionally substituted functional groups such as optionally substituted saturated or unsaturated alkyl, optionally substituted amine, optionally substituted amide, carboxylic acid, succinyl, ester, ether, aldehyde, hydroxyl, halide, thiyl, oxo, sulfoxyl, optionally substituted aryl or optionally substituted heteroaryl functionalities. Most preferably, the fused ring system is a two-ring system or a four ring system. For example, preferred lipophilic tail groups comprise an optionally substituted chromane ring system or an optionally substituted gonane, optionally substituted sterol, optionally substituted sterone, optionally substituted phytosterol, optionally substituted sterane, cholestane or cholestene ring or other related ring system or secosteorid system, for example, comprising three fused 6-membered cyclohexane rings and one fused five membered ring, for example, a cyclopentaperhydrophenanthrene or sterane ring. In one embodiment, the ring system^{RS} is a single heterocyclic ring fused to ring^{a}. Suitably, the heterocyclic ring is a mono-heterocyclic ring comprising a single heteroatom. Preferably O or N. Desirably, the ring system is saturated. More suitably still the ring system^{RS} comprises one substituent R′, which is preferably a C₁-C₆ alkyl group, most preferably methyl.

A preferred anti-agglomerant has the following general structure:

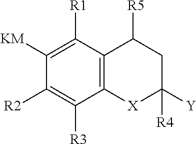

wherein

each X is independently (CH₂)ₙ, where n is 0 to 4, O, N or S;

M is (CH₂)ₙ, where n is 0 to 4, O, N or S:

when M is (CH₂)ₙ and n is 1 to 4, K is selected from the group of functionalities consisting of: optionally substituted alkyl carboxylate, optionally substituted alkyl dicarboxylate, optionally substituted alkyl dicarboxylate ester, optionally substituted alkyl amide, optionally substituted alkyl ether, optionally substituted alkyl ester;

when M is O, K is selected from the group of functionalities consisting of: optionally substituted carboxylate, optionally substituted dicarboxylate, optionally substituted dicarboxylate ester, optionally substituted amide, optionally substituted ether, optionally substituted ester;

when M is N, K is selected from the group of functionalities consisting of: optionally substituted amine, optionally substituted amide, optionally substituted azo, optionally substituted azoxy, optionally substituted carbamate, optionally substituted diazo, optionally substituted diazonium, optionally substituted guanidine, optionally substituted hydrazine, optionally substituted hydrazine, optionally substituted imine; optionally substituted carbonic acid bisamides, optionally substituted oximes, optionally substituted nitrones, and optionally substituted nitriles:

when M is S, K is selected from the group of functionalities consisting of: thiol, selfenic acid, optionally substituted sulfide, optionally substituted disulfide, optionally substituted sulfinic acid, optionally substituted sulfinic acid ester, optionally substituted sulfonic acid, optionally substituted sulfonic acid ester,

wherein the optional substituents are independently selected from one or more of C₁-C₆ alkyl, hydroxyl, acyl, carboxylate, halide, amine or amide functionalities; Y is an optionally substituted branched or unbranched alkyl, an optionally substituted branched or unbranched alkenyl or an optionally substituted branched or unbranched alkynl;

R¹, R², R³, R⁴ and R⁵ are independently selected from H, OR^{x} wherein R^{x} is H or optionally substituted branched or unbranched alkyl, an optionally substituted branched or unbranched alkenyl or an optionally substituted branched or unbranched alkynl, wherein the optional substituents are independently selected from one or more of C₁-C₆ alkyl, hydroxyl, acyl, carboxylate, halide, amine or amide functionalities.

Preferably, M is O, and K is selected from the group of functionalities consisting of: optionally substituted carboxylate, optionally substituted dicarboxylate, optionally substituted dicarboxylate ester, optionally substituted amide, optionally substituted ether, optionally substituted ester, wherein the optional substituents are independently selected from one or more of C₁-C₆ alkyl, hydroxyl, acyl, carboxylate, halide, amine or amide functionalities.

Suitably, K is a carboxylate, alkyl carboxylate, ester or alkyl ester functionality. Preferably, M is O, and the optionally substituted dicarboxylate functionality is of formula —C(O)(L)ₙC(O)OR′, wherein L is a linker group, n is 1 to 10, R⁶ is H or branched or unbranched alkyl.

More preferably. M is O, and the optionally substituted dicarboxylate functionality is selected from oxalate, malonate, succinate, glutarate, adipate, or pimelate.

Most preferably, the optionally substituted dicarboxylate functionality is optionally substituted malonate, succinate or glutarate.

Preferably, X in the ring is O.

More preferably still, Y is an optionally substituted branched or unbranched C₃-C₂₅ alkyl, an optionally substituted branched or unbranched C₃-C₂₅ alkenyl or an optionally substituted branched or unbranched C₃-C₂₅ alkynl. More preferred are optionally substituted branched or unbranched C₅-C₂₀ alkyl, an optionally substituted branched or unbranched C₅-C₂₀ alkenyl or an optionally substituted branched or unbranched C₅-C₂₀ alkynl. Furthermore, preferred are optionally substituted branched or unbranched C₁₂-C₁₇ alkyl, an optionally substituted branched or unbranched C₁₂-C₁₇ alkenyl or an optionally substituted branched or unbranched C₅-C₁₇ alkynl. Most preferred are optionally substituted branched or unbranched C, alkyl, an optionally substituted branched or unbranched C₁₇ alkenyl or an optionally substituted branched or unbranched C₁₇ alkynl. Preferred anti-agglomerants have a saturated C₁₆ alkyl, preferably branched, more preferably branched at carbon 2, 8 and 12 of the C₁₆ alkyl chain.

In most preferred embodiments, any one or more of R¹, R², R³, R⁴ and R⁵ are independently selected from H, methyl, ethyl, propyl or butyl. Preferably, any one or more of R¹, R², R³, R⁴ and R⁵ are independently selected from H and methyl. Most preferably, R¹ is methyl, R² is methyl, R³ is methyl, R⁴ is methyl, and R⁵ is H. Preferably, the anti-agglomerant has the following general structure:

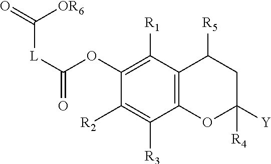

wherein

R¹, R², R³, R⁴ and R⁵ are independently H, OH, methyl, ethyl, propyl or butyl;

L is (CH₂)ₙ alkyl linker and n is 2, 3 or 4 and R⁶ is H;

Y is optionally substituted branched or unbranched C₁₂-C₁₇ alkyl, an optionally substituted branched or unbranched C₁₂-C₁₇ alkenyl or an optionally substituted branched or unbranched C₅-C₁₇ alkynl.

In a particularly preferred embodiment, R¹ is H or methyl, R² is H or methyl, R³ is H or methyl, R⁴ is methyl, and R⁵ is H; L is CH₂ alkyl linker and n is 2, R⁶ is H; and Y is a saturated C₁₄ to C₁₆ alkyl, preferably branched.

When R¹ is H or methyl, R² is H or methyl, R³ is H or methyl, R⁴ is methyl, and R⁵ is H; L is —CH₂-alkyl linker and n is 2, R⁶ is H; and Y is a saturated C₁₄ to C₁₆ alkyl, preferably branched.

When R¹ is H or methyl, R² is H or methyl, R³ is H or methyl, R⁴ is H or methyl, and R⁵ is H; L is —CH₂-alkyl linker and n is 2, Re is H; and Y is a saturated C₁₄ to C₁₆ alkyl.

Suitably, when R¹ is methyl, R² is methyl, R³ is methyl, R⁴ is methyl, and R⁵ is H; and Y is a saturated C₁₆ alkyl having a methyl branch at carbons 2, 8 and 12 of the C₁₆ alkyl chain, the anti-agglomerant is based on alpha-tocopherol. When L is CH₂ alkyl linker and n is 1, R⁶ is H, the anti-agglomerant is alpha-tocopherol succinate.

Suitably, when R¹ is methyl, R² is H, R³ is methyl, R⁴ is methyl, and R⁵ is H; and Y is a saturated C₁₆ alkyl having a methyl branch at carbons 2, 8 and 12 of the C₁₆ alkyl chain, the anti-agglomerant is based on beta-tocopherol. When L is CH₂ alkyl linker and n is 1, R⁶ is H, the anti-agglomerant is beta-tocopherol succinate.

Suitably, when R¹ is H, R² is H, R³ is methyl, R⁴ is methyl, and R⁵ is H; and Y is a saturated C₁₆ alkyl having a methyl branch at carbons 2, 8 and 12 of the C₁₆ alkyl chain, the anti-agglomerant is based on gamma-tocopherol. When L is CH₂ alkyl linker and n is 1, R⁶ is H, the anti-agglomerant is gamma-tocopherol succinate.

Suitably, when R¹ is H, R² is H, R³ is methyl, R⁴ is methyl, and R⁵ is H; and Y is a saturated C₁₆ alkyl having a methyl branch at carbons 2, 8 and 12 of the C₁₆ alkyl chain, the anti-agglomerant is based on delta-tocopherol. When L is CH₂ alkyl linker and n is 1, R⁶ is H, the anti-agglomerant is delta-tocopherol succinate.

Particularly preferred compounds have the following general structure:

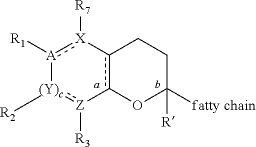

wherein when c is 0, Y is absent, or when c is 1, Y is present, and
   each of A, Y, Z of ring^{a} is independently CH, C, N, O, or S, and each instance of R¹, R², R³ is independently a lone pair or R⁶, provided that when A, Y and Z are independently O or S, the corresponding R¹, R² or R³ is a lone pair, X is CH, C, or N, and
each instance of R⁶, and R⁷ is independently selected from hydrogen, halogen, hydroxyl, ether, alkyl hydroxyl, alkyl ether, oxo, thio, carboxylic acid, carboxylic acid ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted; and
wherein the optional substituents are independently selected from with one or more of H, C₁-C₆ alkyl, hydroxyl, amine, amide or halogen.

Tocopherol due to branching at carbons 2, 8 and 12 of the C₆ alkyl chain has 3 chiral centres and thus eight possible stereoisomers having RRR, RSR, RRS, SRR, SSS, SRS, SSR, or RSS configurations. The RRR stereoisomer is particularly preferred. Most preferred anti-agglomerant molecules are based on the SSS-gamma-tocopherol, for example, SSS-gamma-tocopherol succinate. Racemic mixtures of two or more of these stereoisomers can be used. It will be understood the designation corresponds to the methyl branch at carbons 2, 8 and 12 of the C₁₆ alkyl chain of tocopherol moiety.

Particularly preferred compounds have the following general structure:

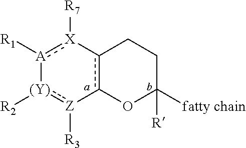

wherein when c is 0, Y is absent, or when c is 1, Y is present, and
   each of A, Y, Z of ring⁹ is independently CH, C, N, O, or S, and each instance of R¹, R², R³ is independently a lone pair or R, provided that when A, Y and Z are independently O or S, the corresponding R¹, R² or R³ is a lone pair,
   X is CH, C, or N, and
   each instance of R⁶, and R⁷ is independently selected from KM, halo, H, OH, OR′, (CH₂)OH, (CH₂)OR′, ═O, C(O)OH, C(O)OR′, (CH₂)ₙC(O)OH, (CH₂)ₙ(O)OR′, NH₂, NHR′, NR′R′, (CH₂)ₙNH₂, (CH₂)ₙNHR′, (CH₂)ₙNR′R′, S(O)₂OH, S(O)₂OR′, (CH₂)ₙS(O)₂OH, (CH₂)ₙS(O)₂OR′, NHS(O)₂OH, NHS(O)₂OR′, (CH₂)ₙNHS(O)₂OH, (CH₂)ₙNHS(O)₂OR′, NH(CH₂)ₙS(O)₂OH, NH(CH₂)ₙS(O)₂OH wherein n is 1 to 20 and R′ is independently selected from the group consisting of H, alkyl, amino, hydroxyl, ether, carboxyl, acyl including ester and aldehyde, a 5- or 6-membered carbocyclic or heterocyclic ring which can be optionally substituted; and wherein KM is selected from the group of functionalities consisting of: alkyl, acyl, carbonyl, carboxyl, alkyl carboxyl, dicarboxyl for example oxalate, malonate, succinate, glutarate, adipate, etc., ester, alkyl ester, diester, dicarboxylate ester, amino, alkyl amino, amido, alkyl amido, ether, alkyl ether, guanidino, alkyl guanidino, imino, alkyl imino, sulfhydryl, sulfonyl, sulfinyl, sulfenyl, phosphoryl, diphosphoryl, thioester, alkyl thioester; or
wherein the optional substituents are independently selected from one or more of C₁-C₆ alkyl, hydroxyl, acyl, carboxyl, halo, amino or amido functionalities.

Tocopherol due to branching at carbons 2, 8 and 12 of the C₁₆ alkyl chain has 3 chiral centres and thus eight possible stereoisomers having RRR, RSR, RRS, SRR, SSS, SRS, SSR, or RSS configurations. The RRR stereoisomer is particularly preferred. Most preferred anti-agglomerant molecules are based on the SSS-gamma-tocopherol, for example, SSS-gamma-tocopherol succinate. Racemic mixtures of two or more of these stereoisomers can be used. It will be understood the designation corresponds to the methyl branch at carbons 2, 8 and 12 of the C₁₆ alkyl chain of tocopherol moiety.

In one embodiment, ring^{a} is an aromatic ring, preferably a heterocyclic or a carbocyclic ring. More suitably, the ring^{a} is a 6 membered aromatic ring, preferably an aromatic heterocyclic or an aromatic carbocyclic ring.

Suitably, ring^{a} is an aromatic ring or heteroaromatic ring having the following general structure:

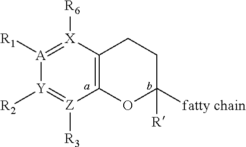

An aromatic carbocyclic ring^{a} is particularly preferred such that the compound is based on a chromane structure. Chromane (benzodihydropyran) is a heterocyclic chemical compound with the chemical formula C₉H₁₀O. Chromane is a structural feature of more complex compounds including E vitamins (tocopherols and tocotrienols). Vitamin E based anti-agglomerants are particularly preferred.

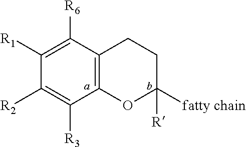

Suitably, at least one of R¹, R³ and R⁶ are independently selected from a C₁-C₆ alkyl group, most preferably methyl, ethyl or butyl. Methyl is particularly preferred.

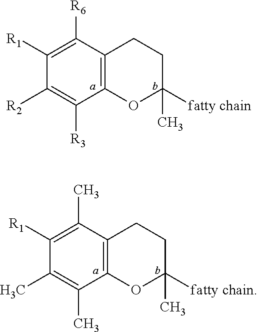

In a particularly preferred embodiment, the anti-agglomerant has the following formula, corresponding to alpha tocopherol succinate:

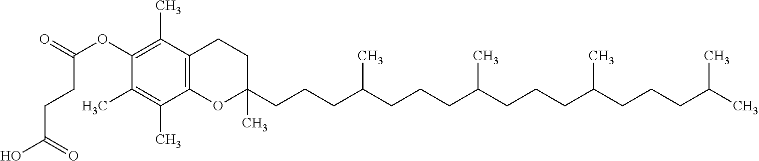 [SMILES: Cc1c(C)c2c(c(C)c1OC(=O)CCC(=O)O)CCC(C)(CCCC(C)CCCC(C)CCCC(C)CCCC(C)C)O2]

In another embodiment, the anti-agglomerant has general formula:

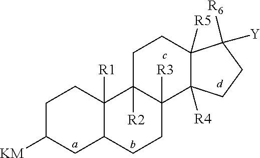

wherein

each of rings a, b, c, d can be independently saturated or unsaturated;

each M is independently (CH₂)ₙ, where n is 0 to 4, O, N or S:

when M is (CH₂)ₙ and n is 1 to 4, K is selected from the group of functionalities consisting of: optionally substituted alkyl carboxylate, optionally substituted alkyl dicarboxylate, optionally substituted alkyl dicarboxylate ester, optionally substituted alkyl amide, optionally substituted alkyl ether, optionally substituted alkyl ester:

when M is O, K is selected from the group of functionalities consisting of: optionally substituted carboxylate, optionally substituted dicarboxylate, optionally substituted dicarboxylate ester, optionally substituted amide, optionally substituted ether, optionally substituted ester;

when M is N, K is selected from the group of functionalities consisting of: optionally substituted amine, optionally substituted amide, optionally substituted azo, optionally substituted azoxy, optionally substituted carbamate, optionally substituted diazo, optionally substituted diazonium, optionally substituted guanidine, optionally substituted hydrazine, optionally substituted hydrazole, optionally substituted imine; optionally substituted carbonic acid bisamides, optionally substituted oximes, optionally substituted nitrones, and optionally substituted nitriles;

when M is S, K is selected from the group of functionalities consisting of: thiol, selfenic acid, optionally substituted sulfide, optionally substituted disulfide, optionally substituted sulfinic acid, optionally substituted sulfinic acid ester, optionally substituted sulfonic acid, optionally substituted sulfonic acid ester,

wherein the optional substituents are independently selected from one or more of C₁-C₆ alkyl, hydroxyl, acyl, carboxylate, halide, amine or amide functionalities;

Y is an optionally substituted branched or unbranched alkyl, an optionally substituted branched or unbranched alkenyl or an optionally substituted branched or unbranched alkynl;

R¹, R², R³, R⁴, R⁵ and R⁷ are independently selected from H, OH, COR^{x}, COOR^{x}, OR^{x} wherein R^{x} is H or optionally substituted branched or unbranched alkyl, an optionally substituted branched or unbranched alkenyl or an optionally substituted branched or unbranched alkynl, wherein the optional substituents are independently selected from one or more of C₁-C₆ alkyl, hydroxyl, acyl, carboxylate, halide, amine or amide functionalities.

Preferably, the anti-agglomerant comprises the following structure:

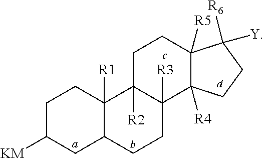

Suitably, M is O.

Where one of the rings is unsaturated, for example, the moiety is a cholestene. For example, ring b is unsaturated at the 5′ and 6′ carbon positions. Preferably, the anti-agglomerant has general formula

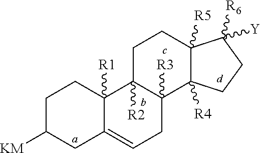

wherein

M is O;

K is selected from the group of functionalities consisting of: optionally substituted carboxylate, optionally substituted dicarboxylate, optionally substituted dicarboxylate ester, optionally substituted amide, optionally substituted ether, optionally substituted ester;

R¹, R², R³, R⁴, R⁵ and R⁶ are independently selected from H, OR^{x} wherein R^{x} is H or optionally substituted branched or unbranched alkyl, an optionally substituted branched or unbranched alkenyl or an optionally substituted branched or unbranched alkynl;

wherein the optional substituents are independently selected from one or more of C₁-C₆ alkyl, hydroxyl, acyl, carboxylate, halide, amine or amide functionalities;

Y is an optionally substituted branched or unbranched alkyl, an optionally substituted branched or unbranched alkenyl or an optionally substituted branched or unbranched alkynl.

More preferably. R², R³, R⁴ and R⁶ are H; R¹ and R⁵ are optionally substituted C₁-C₆ alkyl, preferably methyl, wherein the optional substituents are independently selected from one or more of C₁-C₆ alkyl, hydroxyl, acyl, carboxylate, halide, amine or amide functionalities.

Preferably, Y is an optionally substituted branched or unbranched C₀-C₁₅ alkyl, an optionally substituted branched or unbranched C₀-C₁₅ alkenyl or an optionally substituted branched or unbranched C₀-C₁₅ alkynl.

Further preferably, Y is an optionally substituted branched or unbranched C₅-C₁₂ alkyl, an optionally substituted branched or unbranched C₅-C₁₂ alkenyl or an optionally substituted branched or unbranched C₅-C₁₂ alkynl.

Further preferably, Y is an optionally substituted branched or unbranched C₇-C₁₀ alkyl, an optionally substituted branched or unbranched C₇-C₁₀ alkenyl or an optionally substituted branched or unbranched C₇-C₁₀ alkynl.

Most preferably, Y is an optionally substituted branched or unbranched C₁₀ alkyl, an optionally substituted branched or unbranched C₁₀ alkenyl or an optionally substituted branched or unbranched C₁₀ alkynl. Branched C₁₀ alkyl, or branched C₁₀ alkenyl are particularly preferred.

For example, where Y is a branched C₁₀ alkyl, the anti-agglomerant is based on sitosterol and has the following general structure:

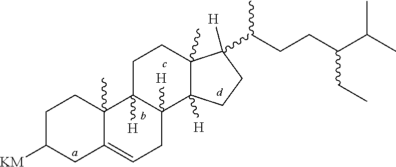

For example, where Y is a branched C₉ alkyl, the anti-agglomerant is based on campesterol and has the following general structure:

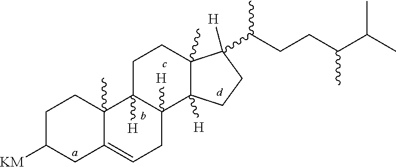

For example, where Y is a branched C₈ alkyl, the anti-agglomerant is based on cholesterol and has the following general structure:

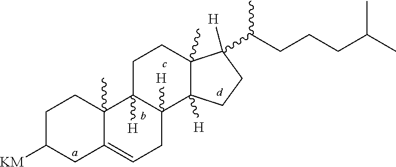

For example, where Y is a branched C₈ alkenyl, the anti-agglomerant is based on sigmasterol and has the following general structure:

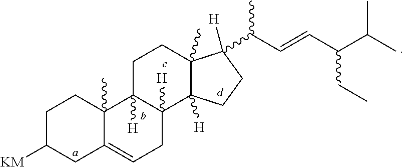

For example, where ring b is saturated and Y is a C₁₀ alkenyl, the anti-agglomerant is based on sigmastanol and has the following general structure:

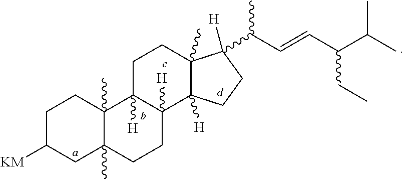

Preferably, K is selected from the group of functionalities consisting of: optionally substituted carboxylate, optionally substituted dicarboxylate, optionally substituted dicarboxylate ester, optionally substituted amide, optionally substituted ether, and optionally substituted ester functionalities.

Particularly preferred compounds have the following general structure:

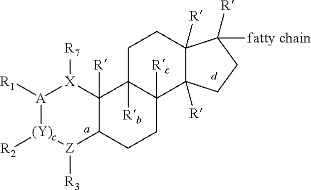

wherein ring^{a} and/or ring^{b} is saturated or unsaturated;
when c is 0, Y is absent, or when c is 1, Y is present, and
each of A, Y, Z of ring^{a} is independently CH, C, N, O, or S, and each instance of R¹, R², R³ is independently a lone pair or R⁶, provided that when A, Y and Z are independently O or S, the corresponding R¹, R² or R³ is a lone pair,
X is CH, C, or N, and
each instance of R⁶, and R⁷ is independently selected from hydrogen, halogen, hydroxyl, ether, alkyl hydroxyl, alkyl ether, oxo, carboxylic acid, carboxylic acid ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted; and wherein the optional substituents are independently selected from with one or more of H, C₁-C₆ alkyl, hydroxyl, amine, amide or halogen.

In a preferred embodiment the anti-agglomerant has the following general structure:

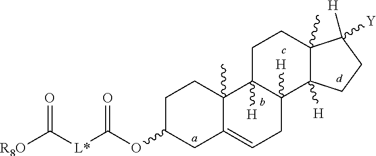

wherein

L* is (CH₂)ₙ alkyl linker and n is 1, 2, 3 or 4, and R⁸ is H; and

Y is an optionally substituted branched C₇-C₁₀ alkyl, or an optionally substituted branched C₇-C₁₀ alkenyl.

More suitably, ring b is unsaturated at the 5′ and 6′ carbon positions.

Most preferable prenol based lipids for the lipophilic tail group of the anti-agglomerant of the invention include Vitamin E [PR202], and vitamin E based substances such as alpha-tocopherol, gamma-CEHC Glc, alpha-tocotrienol, beta-tocotrienol, delta-tocotrienol, gamma-tocotrienol, didesmethyl tocotrienol, beta-Tocopherol, alpha-tocopheronic acid, alpha-tocopheronolactone, gamma-tocopherol and delta-tocopherol.

Suitably, the anti-agglomerant has the following general structure.

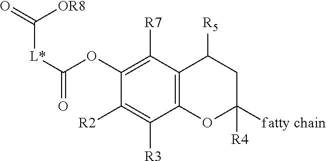

wherein
R², R³, R⁴, R⁵, R⁷ and R⁸ are independently H, OH, methyl, ethyl, propyl or butyl;
L* is (CH₂)ₙ alkyl linker and n is 2, 3 or 4;
fatty chain is optionally substituted branched or unbranched C₁₂-C₁₇ alkyl, an optionally substituted branched or unbranched C₁₂-C₁₇ alkenyl or an optionally substituted branched or unbranched C₅-C₁₇ alkynl.

Suitably, the anti-agglomerant has the following general structure.

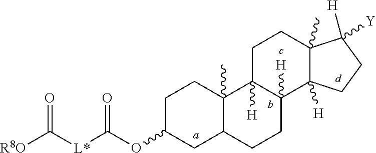

wherein
L is (CH₂)ₙ alkyl linker and n is 1, 2, 3 or 4, and R⁸ is H; and
Y is an optionally substituted branched C₇-C₁₀ alkyl, an optionally substituted branched C₇-C₁₀ alkenyl.

A preferred anti-agglomerant comprises fused ring systems has one of the following generic structures:

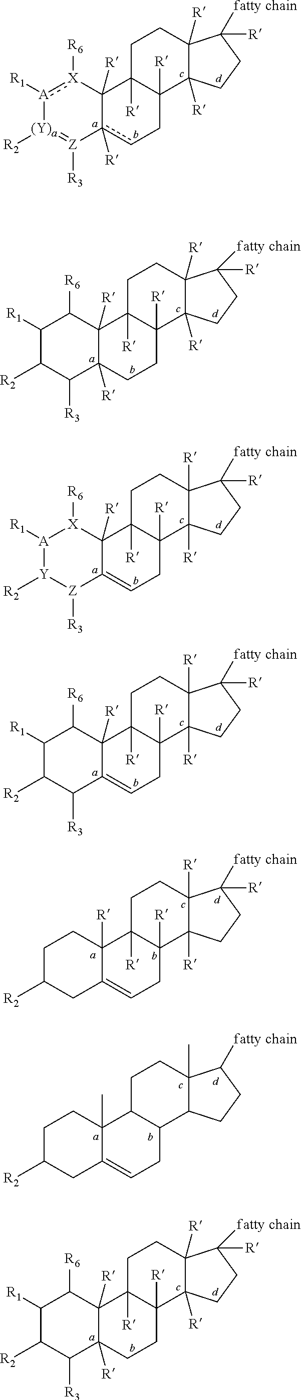

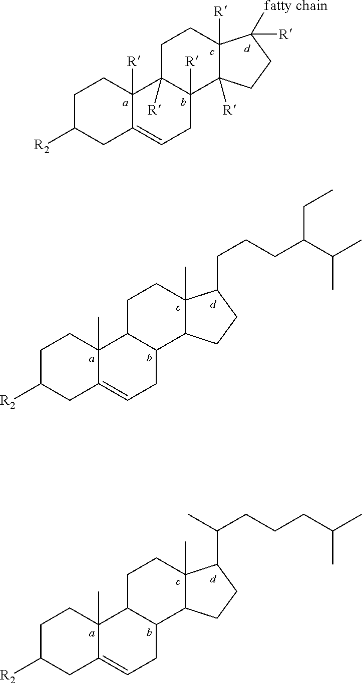

Preferred compounds which may be used as an anti-agglomerant are shown in Table A.

Preferred compounds comprise an aliphatic heterocyclic ring^{a}, exemplified by compounds 13, 15, 17, 26, 27, 28, 30, 31, 34, 35, 37, 38, 41, 43, and 46 of Table A.

Preferred compounds comprise an aromatic carbocyclic ring^{a}, exemplified by compounds 1, 2, 3, 4, 5, 6, 7, 8, 9, 10, 11, 12, 19, 21, 24, 29, 40, 45, 47, 48, 49, 50, 51, 52, 53, 54, 55, 56, 57, 58, 59, 60, 61, 62, 63, 64, 65, 66, 67, 68, 69, 70, 71, 72, 74, 75, 76, 77, 78, 79, 80, 81, 82, 83, 85, 86, and 87 of Table A.

Preferred compounds comprise an aromatic heterocyclic ring^{a}, exemplified by compounds 14, 16, 23, 25, 32, 33, 36, 39, 42, 44, and 84 of Table A.

Preferred compounds comprise have two lipophilic tails, exemplified by compounds 3, 10, 71, and 86 of Table A.

Preferred compounds comprise have fused ring^{a}, exemplified by compounds 11, 14, 16, 18, 73, and 87 of Table A.

Preferred compounds comprise have sulfonic acid based ring^{a}, exemplified by compounds 88, 89, and 90 of Table A.

Particularly preferred compounds include compounds 30, 35, 41, 43 and 48 of Table A.

TABLE A

| | |
|---|---|
| 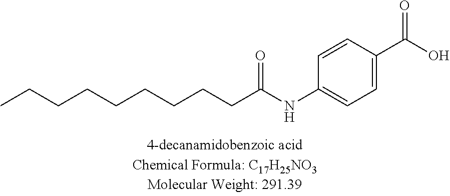 [SMILES: CCCCCCCCCC(=O)Nc1ccc(C(=O)O)cc1] | 1 |
| 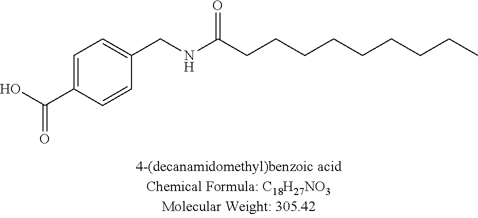 [SMILES: CCCCCCCCCC(=O)NCc1ccc(C(=O)O)cc1] | 2 |
| 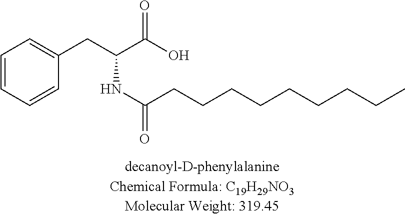 [SMILES: CCCCCCCCCC(=O)N[C@H](Cc1ccccc1)C(=O)O] | 3 |
| 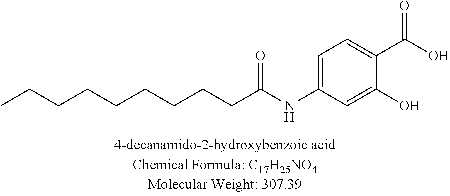 [SMILES: CCCCCCCCCC(=O)Nc1ccc(C(=O)O)c(O)c1] | 4 |
| 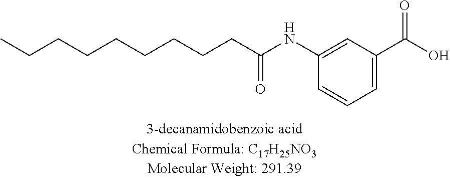 [SMILES: CCCCCCCCCC(=O)Nc1cccc(C(=O)O)c1] | 5 |
| 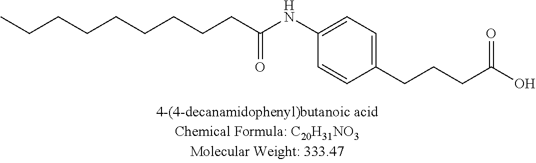 [SMILES: CCCCCCCCCC(=O)Nc1ccc(CCCC(=O)O)cc1] | 6 |
| 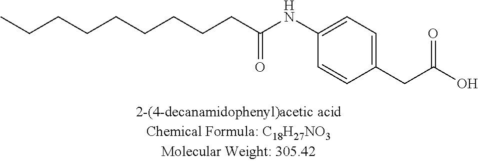 [SMILES: CCCCCCCCCC(=O)Nc1ccc(CC(=O)O)cc1] | 7 |
| 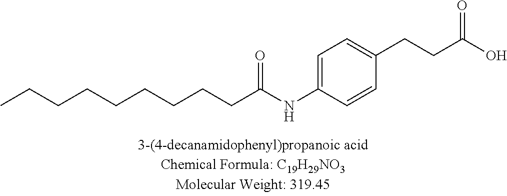 [SMILES: CCCCCCCCCC(=O)Nc1ccc(CCC(=O)O)cc1] | 8 |
| 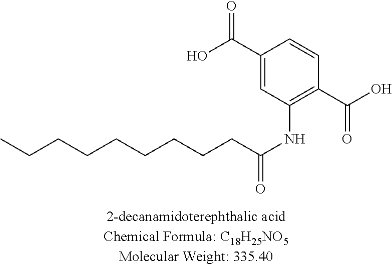 [SMILES: CCCCCCCCCC(=O)Nc1cc(C(=O)O)ccc1C(=O)O] | 9 |
| 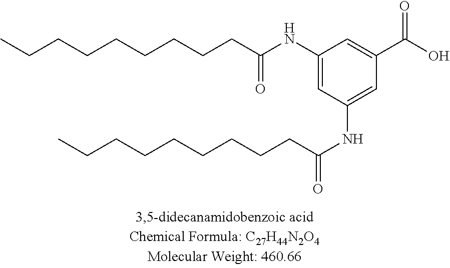 [SMILES: CCCCCCCCCC(=O)Nc1cc(NC(=O)CCCCCCCCC)cc(C(=O)O)c1] | 10 |
| 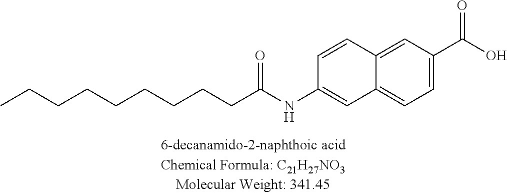 [SMILES: CCCCCCCCCC(=O)Nc1ccc2cc(C(=O)O)ccc2c1] | 11 |
| 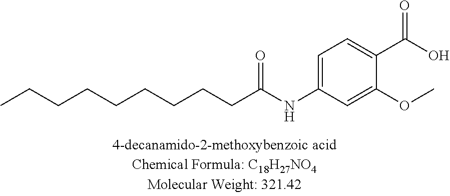 [SMILES: CCCCCCCCCC(=O)Nc1ccc(C(=O)O)c(OC)c1] | 12 |
| 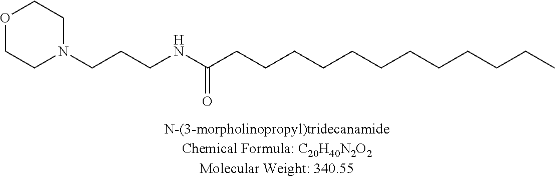 [SMILES: CCCCCCCCCCCCC(=O)NCCCN1CCOCC1] | 13 |
| 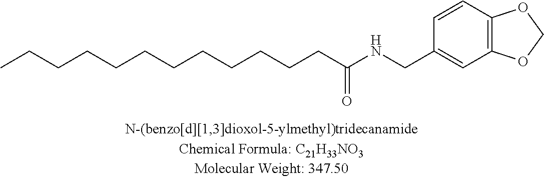 [SMILES: CCCCCCCCCCCCC(=O)NCc1ccc2c(c1)OCO2] | 14 |
| 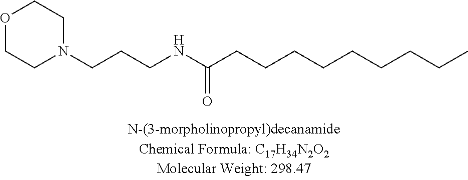 [SMILES: CCCCCCCCCC(=O)NCCCN1CCOCC1] | 15 |
| 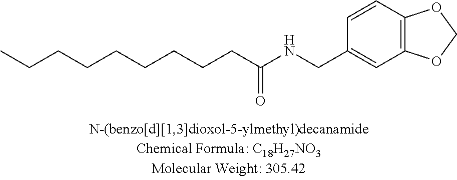 [SMILES: CCCCCCCCCC(=O)NCc1ccc2c(c1)OCO2] | 16 |
| 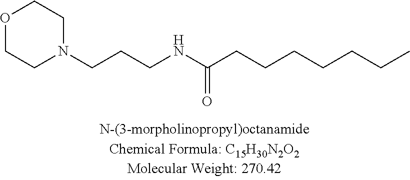 [SMILES: CCCCCCCC(=O)NCCCN1CCOCC1] | 17 |
| 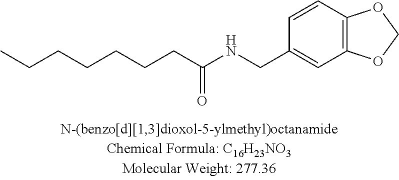 [SMILES: CCCCCCCC(=O)NCc1ccc2c(c1)OCO2] | 18 |
| 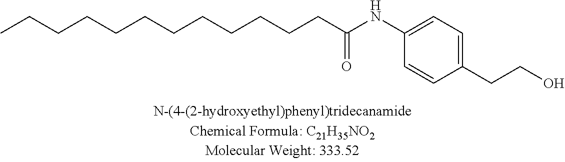 [SMILES: CCCCCCCCCCCCC(=O)Nc1ccc(CCO)cc1] | 19 |
| 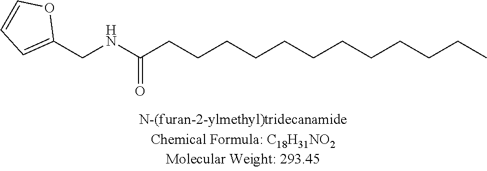 [SMILES: CCCCCCCCCCCCC(=O)NCc1ccco1] | 20 |
| 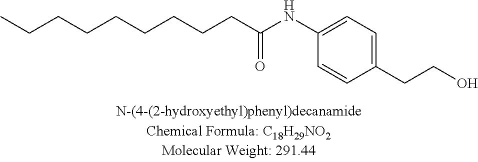 [SMILES: CCCCCCCCCC(=O)Nc1ccc(CCO)cc1] | 21 |
| 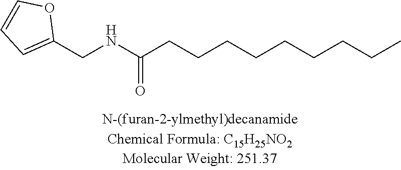 [SMILES: CCCCCCCCCC(=O)NCc1ccco1] | 23 |
| 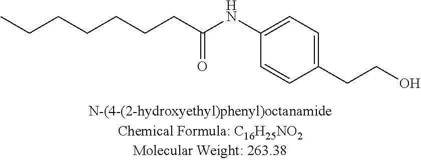 [SMILES: CCCCCCCC(=O)Nc1ccc(CCO)cc1] | 24 |
| 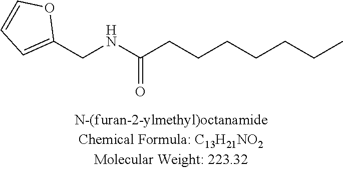 [SMILES: CCCCCCCC(=O)NCc1ccco1] | 25 |
| 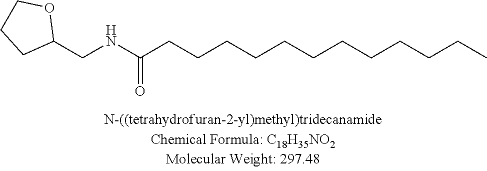 [SMILES: CCCCCCCCCCCCC(=O)NCC1CCCO1] | 26 |
| 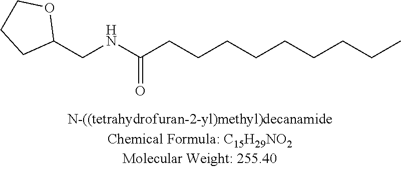 [SMILES: CCCCCCCCCC(=O)NCC1CCCO1] | 27 |
| 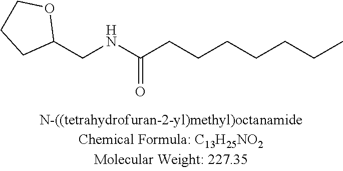 [SMILES: CCCCCCCC(=O)NCC1CCCO1] | 28 |
| 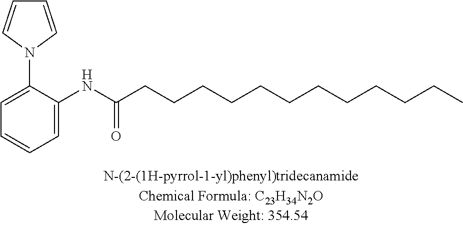 [SMILES: CCCCCCCCCCCCC(=O)Nc1ccccc1-n1cccc1] | 29 |
| 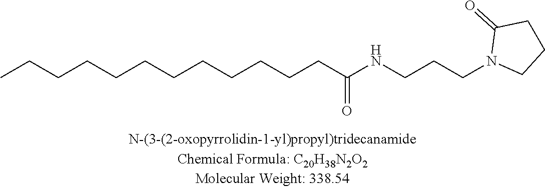 [SMILES: CCCCCCCCCCCCC(=O)NCCCN1CCCC1=O] | 30 |
| 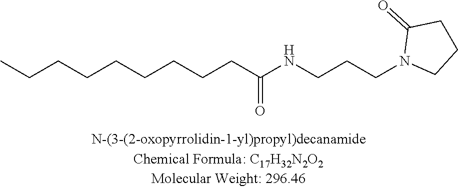 [SMILES: CCCCCCCCCC(=O)NCCCN1CCCC1=O] | 31 |
| 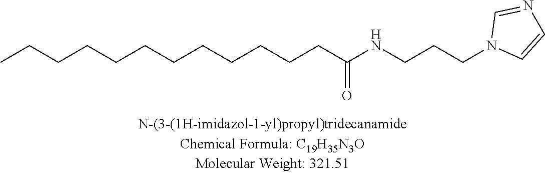 [SMILES: CCCCCCCCCCCCC(=O)NCCCn1ccnc1] | 32 |
| 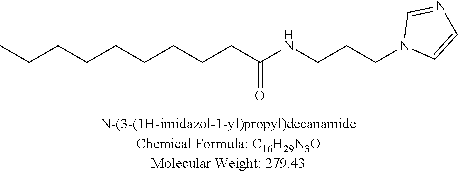 [SMILES: CCCCCCCCCC(=O)NCCCn1ccnc1] | 33 |
| 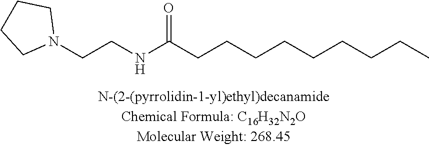 [SMILES: CCCCCCCCCC(=O)NCCN1CCCC1] | 34 |
| 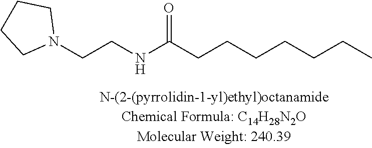 [SMILES: CCCCCCCC(=O)NCCN1CCCC1] | 35 |
| 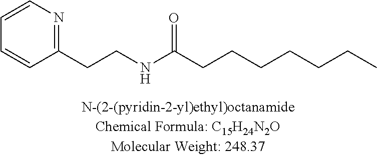 [SMILES: CCCCCCCC(=O)NCCc1ccccn1] | 36 |
| 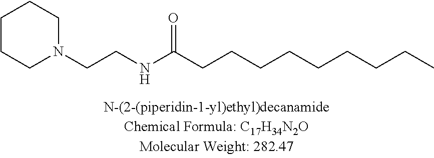 [SMILES: CCCCCCCCCC(=O)NCCN1CCCCC1] | 37 |
| 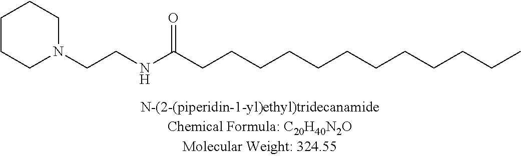 [SMILES: CCCCCCCCCCCCC(=O)NCCN1CCCCC1] | 38 |
| 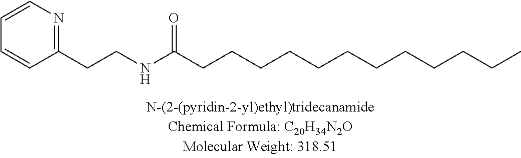 [SMILES: CCCCCCCCCCCCC(=O)NCCc1ccccn1] | 39 |
| 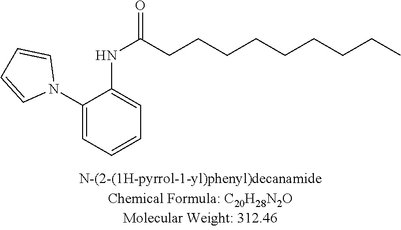 [SMILES: CCCCCCCCCC(=O)Nc1ccccc1-n1cccc1] | 40 |
| 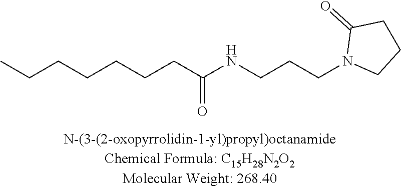 [SMILES: CCCCCCCC(=O)NCCCN1CCCC1=O] | 41 |
| 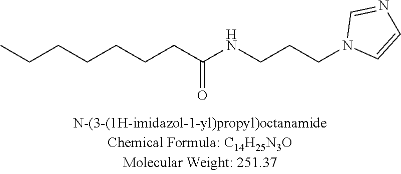 [SMILES: CCCCCCCC(=O)NCCCn1ccnc1] | 42 |
| 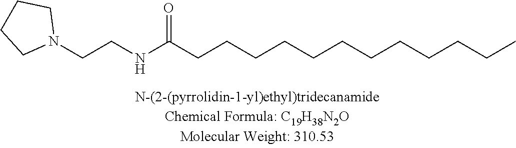 [SMILES: CCCCCCCCCCCCC(=O)NCCN1CCCC1] | 43 |
| 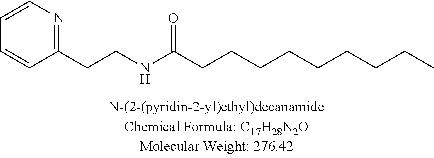 [SMILES: CCCCCCCCCC(=O)NCCc1ccccn1] | 44 |
| 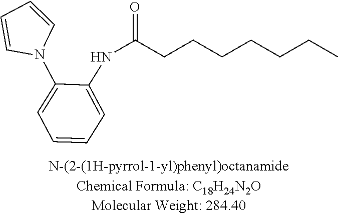 [SMILES: CCCCCCCC(=O)Nc1ccccc1-n1cccc1] | 45 |
| 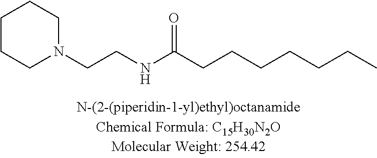 [SMILES: CCCCCCCC(=O)NCCN1CCCCC1] | 46 |
| 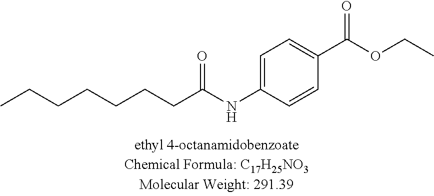 [SMILES: CCCCCCCC(=O)Nc1ccc(C(=O)OCC)cc1] | 47 |
| 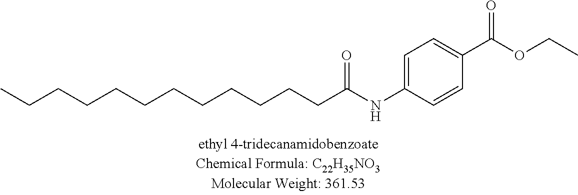 [SMILES: CCCCCCCCCCCCC(=O)Nc1ccc(C(=O)OCC)cc1] | 48 |
| 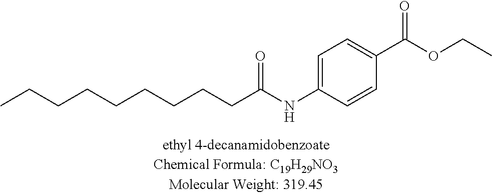 [SMILES: CCCCCCCCCC(=O)Nc1ccc(C(=O)OCC)cc1] | 49 |
| 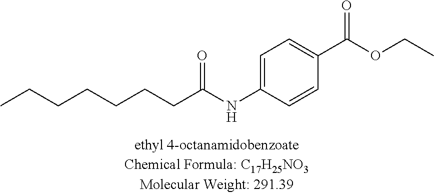 [SMILES: CCCCCCCC(=O)Nc1ccc(C(=O)OCC)cc1] | 50 |
| 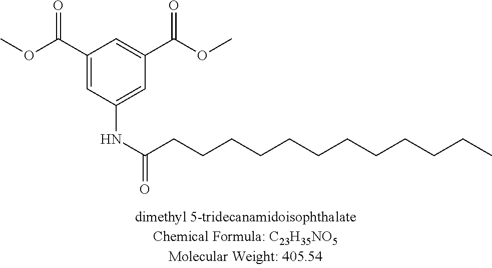 [SMILES: CCCCCCCCCCCCC(=O)Nc1cc(C(=O)OC)cc(C(=O)OC)c1] | 51 |
| 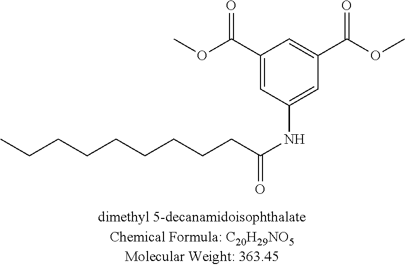 [SMILES: CCCCCCCCCC(=O)Nc1cc(C(=O)OC)cc(C(=O)OC)c1] | 52 |
| 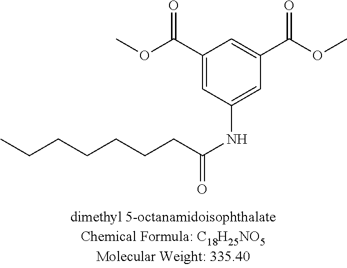 [SMILES: CCCCCCCC(=O)Nc1cc(C(=O)OC)cc(C(=O)OC)c1] | 53 |
| 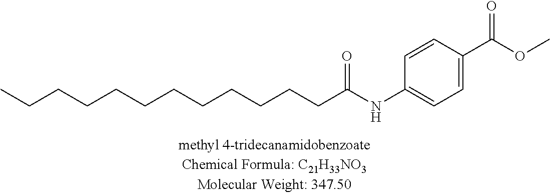 [SMILES: CCCCCCCCCCCCC(=O)Nc1ccc(C(=O)OC)cc1] | 54 |
| 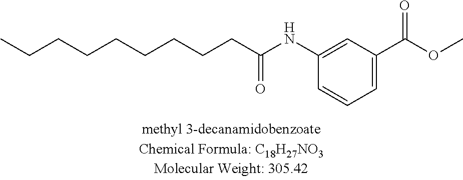 [SMILES: CCCCCCCCCC(=O)Nc1cccc(C(=O)OC)c1] | 55 |
| 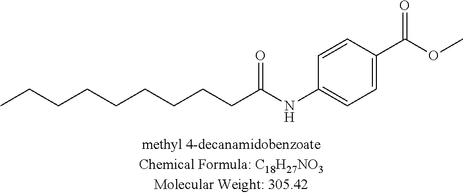 [SMILES: CCCCCCCCCC(=O)Nc1ccc(C(=O)OC)cc1] | 56 |
| 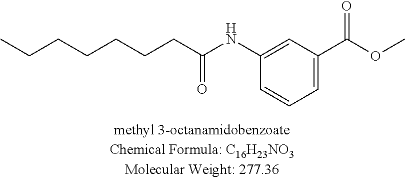 [SMILES: CCCCCCCC(=O)Nc1cccc(C(=O)OC)c1] | 57 |
| 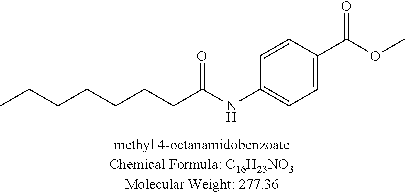 [SMILES: CCCCCCCC(=O)Nc1ccc(C(=O)OC)cc1] | 58 |
| 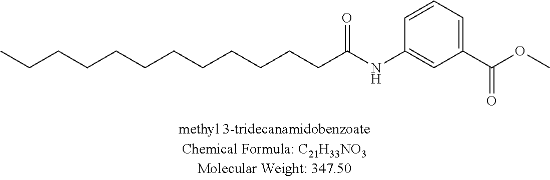 [SMILES: CCCCCCCCCCCCC(=O)Nc1cccc(C(=O)OC)c1] | 59 |
| 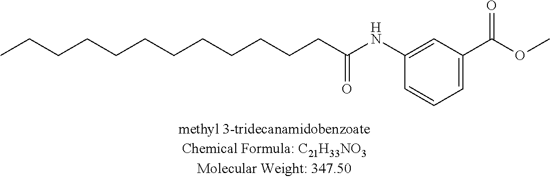 [SMILES: CCCCCCCCCCCCC(=O)Nc1cccc(C(=O)OC)c1] | 60 |
| 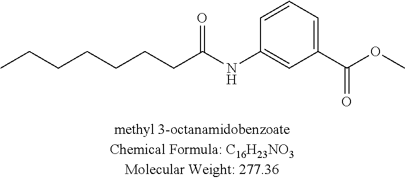 [SMILES: CCCCCCCC(=O)Nc1cccc(C(=O)OC)c1] | 61 |
| 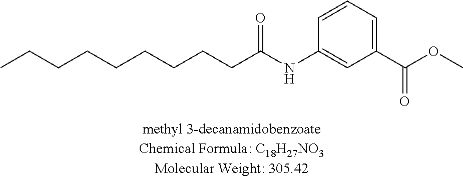 [SMILES: CCCCCCCCCC(=O)Nc1cccc(C(=O)OC)c1] | 62 |
| 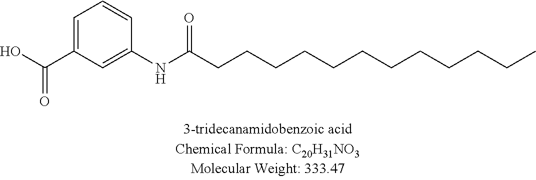 [SMILES: CCCCCCCCCCCCC(=O)Nc1cccc(C(=O)O)c1] | 63 |
| 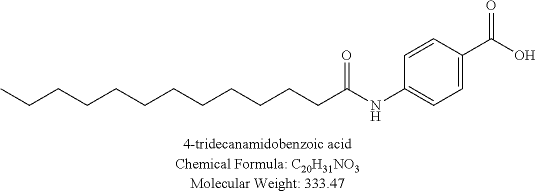 [SMILES: CCCCCCCCCCCCC(=O)Nc1ccc(C(=O)O)cc1] | 64 |
| 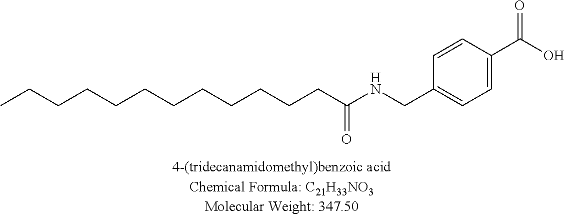 [SMILES: CCCCCCCCCCCCC(=O)NCc1ccc(C(=O)O)cc1] | 65 |
| 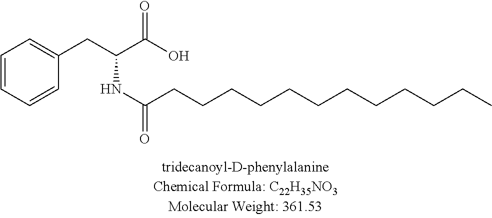 [SMILES: CCCCCCCCCCCCC(=O)N[C@H](Cc1ccccc1)C(=O)O] | 66 |
| 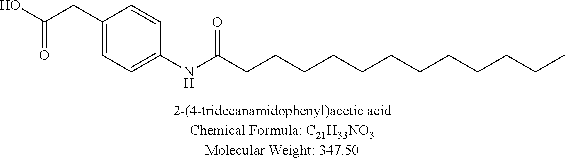 [SMILES: CCCCCCCCCCCCC(=O)Nc1ccc(CC(=O)O)cc1] | 67 |
| 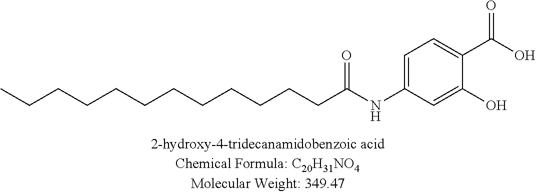 [SMILES: CCCCCCCCCCCCC(=O)Nc1ccc(C(=O)O)c(O)c1] | 68 |
| 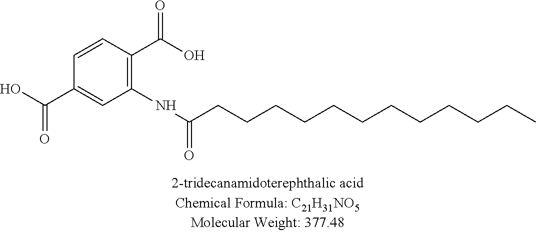 [SMILES: CCCCCCCCCCCCC(=O)Nc1cc(C(=O)O)ccc1C(=O)O] | 69 |
| 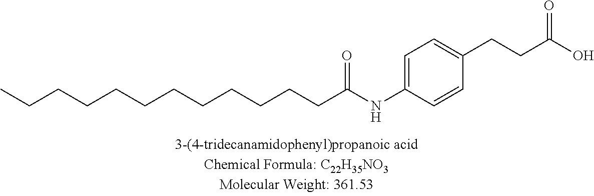 [SMILES: CCCCCCCCCCCCC(=O)Nc1ccc(CCC(=O)O)cc1] | 70 |
| 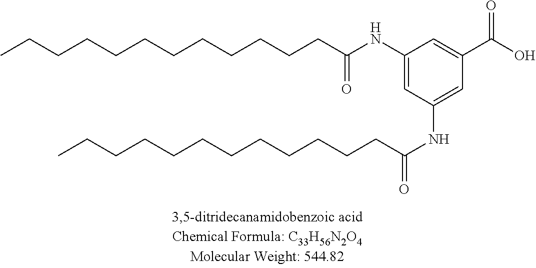 [SMILES: CCCCCCCCCCCCC(=O)Nc1cc(NC(=O)CCCCCCCCCCCC)cc(C(=O)O)c1] | 71 |
| 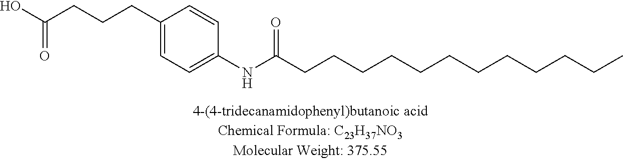 [SMILES: CCCCCCCCCCCCC(=O)Nc1ccc(CCCC(=O)O)cc1] | 72 |
| 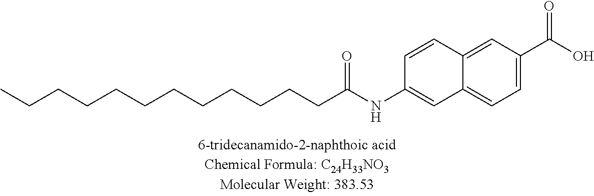 [SMILES: CCCCCCCCCCCCC(=O)Nc1ccc2cc(C(=O)O)ccc2c1] | 73 |
| 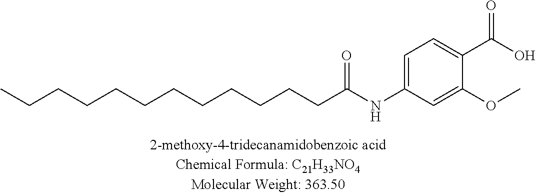 [SMILES: CCCCCCCCCCCCC(=O)Nc1ccc(C(=O)O)c(OC)c1] | 74 |
| 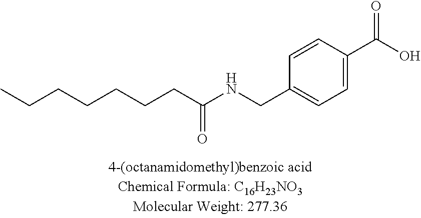 [SMILES: CCCCCCCC(=O)NCc1ccc(C(=O)O)cc1] | 75 |
| 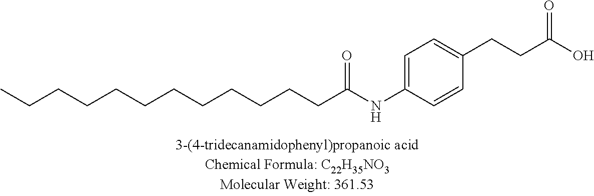 [SMILES: CCCCCCCCCCCCC(=O)Nc1ccc(CCC(=O)O)cc1] | 76 |
| 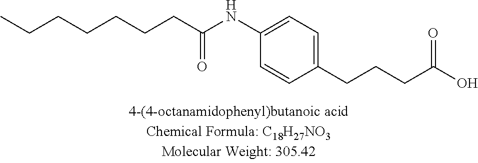 [SMILES: CCCCCCCC(=O)Nc1ccc(CCCC(=O)O)cc1] | 77 |
| 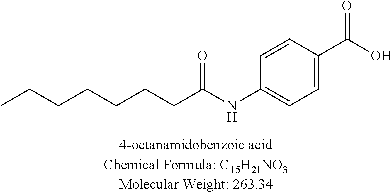 [SMILES: CCCCCCCC(=O)Nc1ccc(C(=O)O)cc1] | 78 |
| 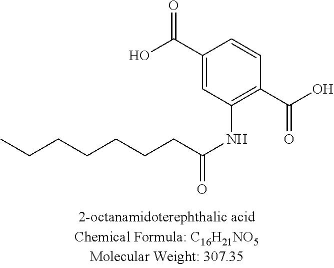 [SMILES: CCCCCCCC(=O)Nc1cc(C(=O)O)ccc1C(=O)O] | 79 |
| 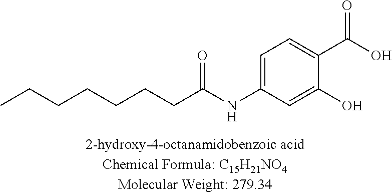 [SMILES: CCCCCCCC(=O)Nc1ccc(C(=O)O)c(O)c1] | 80 |
| 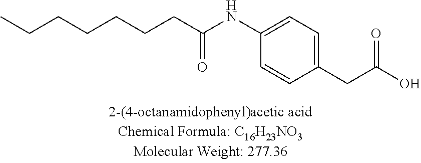 [SMILES: CCCCCCCC(=O)Nc1ccc(CC(=O)O)cc1] | 81 |
| 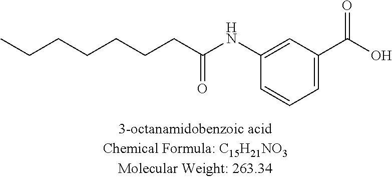 [SMILES: CCCCCCCC(=O)Nc1cccc(C(=O)O)c1] | 82 |
| 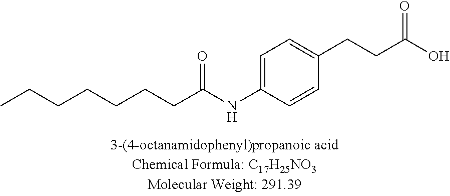 [SMILES: CCCCCCCC(=O)Nc1ccc(CCC(=O)O)cc1] | 83 |
| 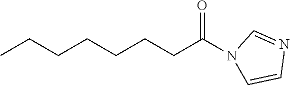 | 84 |
| 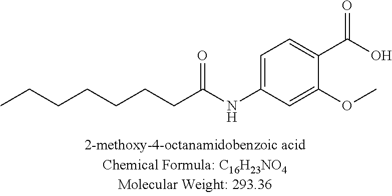 [SMILES: CCCCCCCC(=O)Nc1ccc(C(=O)O)c(OC)c1] | 85 |
| 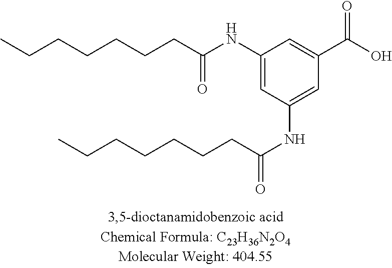 [SMILES: CCCCCCCC(=O)Nc1cc(NC(=O)CCCCCCC)cc(C(=O)O)c1] | 86 |
| 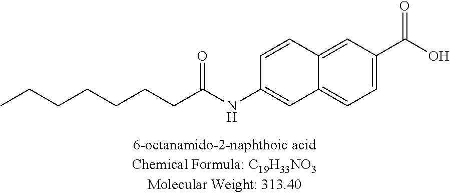 [SMILES: CCCCCCCC(=O)Nc1ccc2cc(C(=O)O)ccc2c1] | 87 |
| 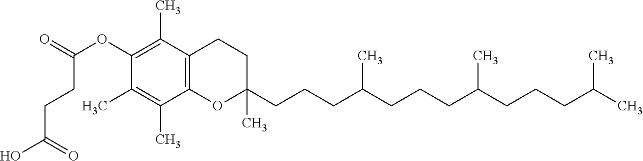 [SMILES: Cc1c(C)c2c(c(C)c1OC(=O)CCC(=O)O)CCC(C)(CCCC(C)CCCC(C)CCCC(C)C)O2] | 88 |
| 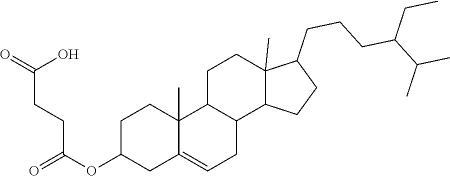 [SMILES: CCC(CCCC1CCC2C3CC=C4CC(OC(=O)CCC(=O)O)CCC4(C)C3CCC12C)C(C)C] | 89 |
| 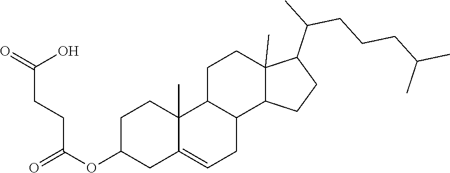 [SMILES: CC(C)CCCC(C)C1CCC2C3CC=C4CC(OC(=O)CCC(=O)O)CCC4(C)C3CCC12C] | 90 |

A particularly preferred compound is selected from the group consisting of:

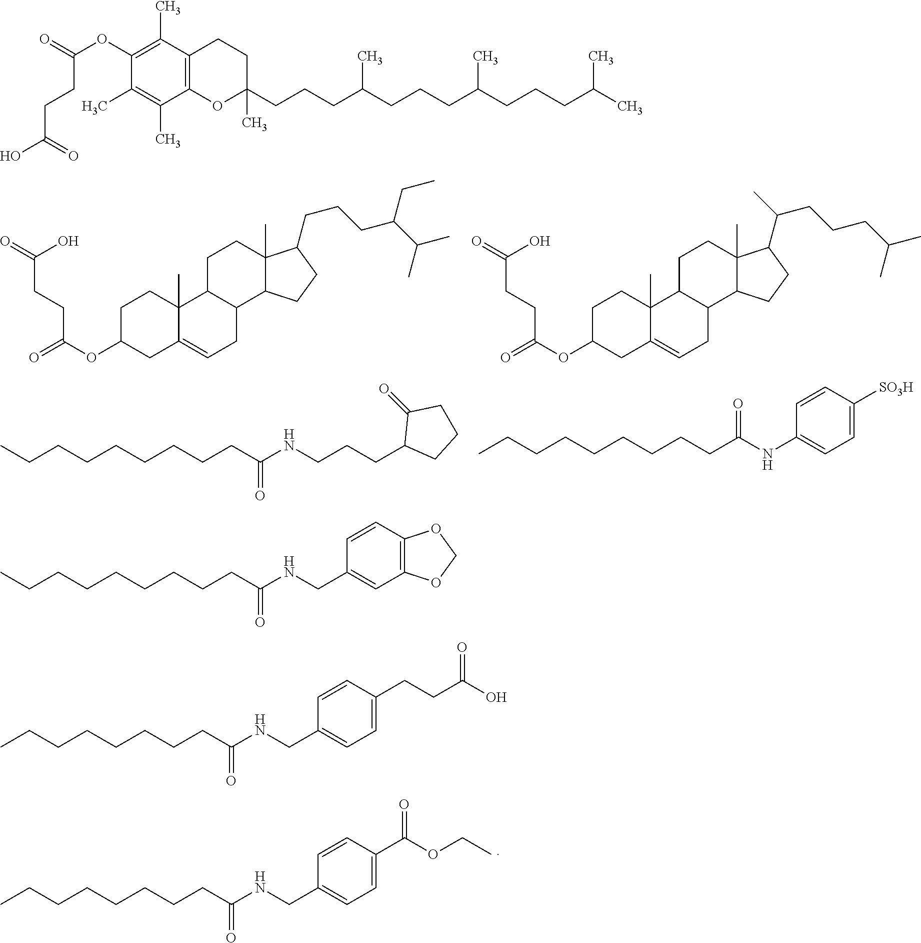

Suitably, at least one of the at least one non-ionic hydrophilic head group component and the at least one lipophilic tail component are derived from a naturally occurring product, a precursor to, a metabolite or a derivative thereof.

Preferred compounds are biodegradable in the sense that they produce a score of >20%, more preferably, >60% biodegradability over 28 days as assessed by OECD 306: Biodegradability in Seawater, the Closed Bottle Test.

Preferred compounds have an ecotoxicity of LC50/EC50 of greater than 10 mg/L as determined by OECD 203: Fish, Acute Toxicity Test.

Bioaccumulation potential determined by HPLC for water soluble chemicals and the shake-flask method (OECD 107) for more lipophilic compounds. Compounds having a log P_{ow} of >4.5-5 are considered to be potentially highly bio accumulative. Preferred compounds have Most preferred compounds meet all of the above criteria.

Particularly preferred compounds are oil soluble.

In another aspect, the invention provides a use of a precursor compound in the manufacture of a compound as described herein, wherein the non-ionic hydrophilic head group component is derived from a precursor compound comprising a non-sugar alcohol or a non-sugar ester 5- or 6-membered carbocyclic or heterocyclic ring substituted with at least one functional group couplable to at least one lipophilic tail component comprising a lipophilic tail.

Suitable precursor compounds and lipophilic tails have been described above.

In a further aspect, the invention provides a compound obtainable by a use or the method as described herein.

In one aspect, the invention provides the use of compound as described herein as an anti-agglomerant and/or as a corrosion inhibitor.

Preferably, the use is the prevention of gas hydrate cluster formation.

Suitably, the anti-agglomerant and/or corrosion inhibitor is used in the presence of a hydrocarbon phase, for example, in hydrocarbon transport, in oil and/or natural gas recovery, particularly where the hydrocarbon phase is transported in cold conditions, both onshore and offshore, preferably, as an alternative or supplement to thermodynamic inhibitors (MEG) in applications where thermodynamic inhibitors are used. In a related aspect, the invention provides a composition comprising a compound as described herein, for corrosion inhibition.

### DETAILED DESCRIPTION

The inventors have found that anti-agglomerant molecules based on naturally occurring substances, such as a tocopherol (vitamin E) based inhibitor (anti-agglomerant) can be used to prevent gas hydrate agglomeration.

The tocopherol based anti-agglomerant has been found to be gas hydrate selective such that the chemical can be dosed at low concentrations. The tocopherol based anti-agglomerants are derived from natural sources and so is environmentally benign. The performance of the anti-agglomerant of the invention is comparable to known AAs without carrying the quaternary ammonium group which is environmentally unacceptable.

Furthermore, the physical structure of the gas hydrate formed in the presence of the anti-agglomerant of the invention is similar to that formed when conventional AAs are used.

The anti-agglomerant of the invention is oil soluble so partitioning into aqueous phase is reduced. Therefore the anti-agglomerant can assist with overboard water quality despite the fact the compound is benign. The method generally comprises addition of the anti-agglomerant into a hydrocarbon fluid. The anti-agglomerant may be added neat or may be added as a solution or dispersion in a suitable carrier. Examples of liquid carriers may be selected from the group consisting of water, brine, seawater, methanol, ethanol, propanol, isopropanol, monoethylene glycol and mixtures thereof. If desired the polymeric hydrate inhibitor may be added to the hydrocarbon fluid in admixture with or contemporaneously with a conventional thermodynamic hydrate inhibitor such as methanol, ethanol, propanol, isopropanol, monoethylene glycol and mixtures thereof. The anti-agglomerant in combination with the conventional thermodynamic hydrate inhibitors and/or conventional AAs will generally provide much more effective control of hydrate formation and/or will allow a significant reduction in the use of the conventional hydrate inhibitor and/or AA.

The amount of the anti-agglomerant added to the hydrocarbon fluid well depend on the nature and conditions such as pressure and temperature under which the hydrocarbon fluid is transported and the amount of water present in the hydrocarbon fluid. The amount will generally be chosen to be effective to inhibit hydrate formation such that plug formation is avoided. The amount is typically in the range of from 0.01% to 5% by weight based on the weight of water in the hydrocarbon fluid and preferably in the range of from 0.01% to 3% such as 0.01% to 2% by weight based on the weight of water in the hydrocarbon fluid.

The anti-agglomerant may be used in controlling the formation of hydrocarbon hydrates in a range of hydrocarbons. Examples of hydrocarbons in which hydrate formation is a particular problem include, but are not limited to, crude oil, methane, ethane, propane, isobutane, butane, neopentane, ethylene, propylene, isobutylene, cyclopropane, cyclobutane, cyclopentane, cyclohexane, acetylene, methylacetylene and benzene.

The process using the anti-agglomerant of the invention is particularly useful in inhibiting agglomeration of gas hydrates into dusters of a size that can plug, block or impede flow through a line through which a hydrocarbon is transported.

The hydrate anti-agglomerants of the invention also generally have the further particularly desirable attribute of acting as corrosion inhibitors. Generally, it has been the practice to use corrosion inhibitors in combination with hydrate inhibitors. Corrosion inhibitors are in some cases needed to compensate for corrosive effect of compositions containing the hydrate inhibitor and in some cases the hydrate inhibitors (particularly those of the KHI type) have been found to interact with and counteract the corrosion inhibitor. Examples of known corrosion inhibitors include primary, secondary or tertiary amines or quaternary ammonium salts, preferably amines or salts containing at least one lipophilic group, benzalkonium halides, preferably benzyl hexyldimethyl ammonium chloride.

Preferably, the anti-agglomerant of the invention provides both hydrate inhibition and metal corrosion inhibition in a single additive.

Accordingly, there is provided a method of inhibiting hydrate agglomeration and inhibiting corrosion produced in a hydrocarbon phase comprising water when in contact with metals, particularly ferrous metals, the method comprising adding to the hydrocarbon phase, an anti-agglomerant as described herein. Suitably, the anti-agglomerant is added in an amount sufficient to inhibit gas hydrate formation and to inhibit corrosion of the metal component.

The amount necessary to inhibit corrosion of the metal component will depend on the metal present in the metal component, nature an proportion of components in the phase and the conditions such as temperature and pressure under which the phase is in contact with the metal. The amount will generally be at least 0.01% based on the water content of the phase, preferably at least 0.1%. In one set of embodiments the amount of inhibitor s in the range of from 0.01% to 5% by weight based on the weight of water in the hydrocarbon fluid and preferably in the range of from 0.01% to 3% such as 0.01% to 2% by weight based on the weight of water in the hydrocarbon fluid.

The corrosion inhibition provided by the hydrate anti-agglomerants described herein is particularly useful in pumping a hydrocarbon phase in a metal (particularly ferrous metal) line, e.g., conduit under conditions of temperature and pressure which would, in the absence of the hydrate anti-agglomerant, lead to formation of hydrocarbon hydrates.

### BRIEF DESCRIPTION OF DRAWINGS

In the attached drawings:

FIG. 1 illustrates the chemical structures of a number of commercially available tocopherol samples;

FIG. 2 illustrates the chemical structure of one green anti-agglomerant, tocopherol succinate, of the invention (anti-agglomerant1) and chemical structure of a conventional anti-agglomerant, dodecyltrimethylammonium bromide;

FIG. 3 illustrates a comparison by overlay of the change in hydrate volume fraction over time in each of the pure water, anti-agglomerant and commercial AA systems averaged over 10 runs;

FIG. 4 illustrates an overlay of a representative cycle of the hydrate fraction against relative torque for each of the pure water, anti-agglomerant and commercial AA systems;

FIG. 5(a) illustrates a representative plot of hydrate growth rate in pure water (system 1) for one experimental cycle in which the hydrate volume fraction is plotted as a function of time; FIG. 5(b) illustrates the average hydrate growth rate for each stage of the experiment run in pure water, whereby the representative rate is represented by a triangle symbol:

FIG. 6 illustrates the hydrate formation kinetic data collected in pure water (system 1) over the 10 experiments carried out, as well as the calculated average data for all experiments;

FIG. 7 illustrates the hydrate characteristics of gas consumption, hydrate volume fraction and water conversion for each experiment in pure water (system 1);

FIG. 8 illustrates the hydrate growth rate per stage for each experiment in pure water (system 1), as well as the calculated average for all experiments;

FIG. 9 illustrates a representative plot of hydrate growth rate in water and anti-agglomerant (system 2) for one experimental cycle in which the hydrate volume fraction is plotted as a function of time;

FIG. 9(b) illustrates the average hydrate growth rate for each stage of the experiment run in water and anti-agglomerant (system 2), whereby the representative rate is represented by a triangle symbol:

FIG. 10 illustrates the hydrate formation kinetic data collected in water and anti-agglomerant (system 2) over the 10 experiments carried out, as well as the calculated average data for all experiments;

FIG. 11 illustrates the hydrate characteristics of gas consumption, hydrate volume fraction and water conversion for each experiment in water and anti-agglomerant (system 2);

FIG. 12 illustrates the hydrate growth rate per stage for each experiment in water and anti-agglomerant (system 2) as well as the calculated average for all experiments;

FIG. 13 illustrates a representative plot of hydrate growth rate in water and conventional AA (system 3) for one experimental cycle in which the hydrate volume fraction is plotted as a function of time;

FIG. 13(b) illustrates the average hydrate growth rate for each stage of the experiment run in water and conventional AA (system 3), whereby the representative rate is represented by a triangle symbol;

FIG. 14 illustrates the hydrate formation kinetic data collected in water and conventional AA (system 3)) over the 10 experiments carried out, as well as the calculated average data for all experiments;

FIG. 15 illustrates the hydrate characteristics of gas consumption, hydrate volume fraction and water conversion for each experiment in water and conventional AA (system 3);

FIG. 16 illustrates the hydrate growth rate per stage for each experiment in water and anti-agglomerant (system 3) as well as the calculated average for all experiments;

FIG. 17(a) illustrates the X-ray diffraction pattern of the gas hydrates formed in systems 1-3. FIG. 17(b) represents an overlay of the respective X-ray diffraction patterns of FIG. 17(a) demonstrating the similarly in gas hydrate structure formed in each of system 1-3;

FIG. 18 illustrates the X-ray diffraction peaks identifying the presence of the solid decane phase, the SI and SII hydrate phases and small levels of hexagonal ice phase in the hydrates formed in system 1;

FIG. 19 illustrates the X-ray diffraction peaks identifying the presence of the solid decane phase, the SI and SII hydrate phases in the hydrates formed in system 2; and

FIG. 20 illustrates the X-ray diffraction peaks identifying the presence of the solid decane phase, the SI and SII hydrate phases in the hydrates formed in system 2.

### DETAILED DESCRIPTION OF THE INVENTION

The invention will now be described with reference to the following examples. It is to be understood that the examples are provided by way of illustration of the invention and that they are in no way limiting to the scope of the invention.

### Examples

The following anti-agglomerants have been synthesised and characterised:

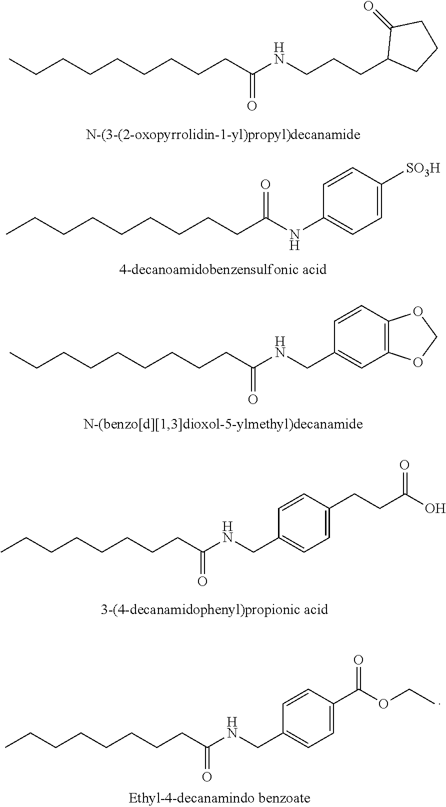 [SMILES: CCCCCCCCCC(=O)NCCCC1CCCC1=O ; CCCCCCCCCC(=O)Nc1ccc(S(=O)(=O)O)cc1 ; CCCCCCCCCC(=O)NCc1ccc2c(c1)OCO2 ; CCCCCCCCC(=O)NCc1ccc(CCC(=O)O)cc1 ; CCCCCCCCC(=O)NCc1ccc(C(=O)OCC)cc1]

### Exemplary Synthesis Involving Amine and Fatty Carboxyl Coupling

Alkanoic acid (1 molar equivalence) was dissolved into anhydrous ethyl acetate under inert atmosphere at room temperature. N,N′-carbonyl diimidazole (CDI) (1 molar equivalence) was added to the alkanoic acid and stirred for 1-2 hours. Then the amine (1.1-1.2 molar equivalence) was added and the reaction was heated to 80° C., under reflux. The reaction was allowed to react for 24 hours at 80° C. before cooling to room temperature. Work-up involved washing the reaction mixture with 1M hydrochloric acid (50 mL, 3 times), 1M sodium hydrogen carbonate (50 mL, twice) and finally saturated sodium chloride. Ethyl acetate was taken off under vacuum to afford the purified product.

### Gas Hydrate Testing

A high pressure autoclave equipped with a magnetic stirrer coupling was used to study hydrate formation. This provides information regarding the hydrate onset time, growth rate, and hydrate fraction by measuring pressure, temperature, and torque changes during hydrate formation. A synthetic natural gas mixture was used in all of the experiments according to Table 3.

TABLE 3
Composition of synthetic natural gas

| Components | Composition (mol %) |
|---|---|
| CH₄ | 90 |
| C₂H₆ | 6 |
| C₃H₈ | 3 |
| n-C₄H₁₀ | 1 |

Decane is added to the test as a continuous phase, the gas mixture does not contain a liquid condensate.

### Conditions

Total liquid of 80 ml (0.5 wt % tocopherol AA and commercial AA solution), and pure water in 360 ml internal volume autoclave under watercut 30 environment
120 bar natural gas saturation (methane: 90 mol %, ethane: 6 mol %, propane: 3 mol %, n-butane: 1 mol %) at 24° C., with 600 rpm stirring
Cooling rate: 0.25 Kmin⁻¹ (24→4° C.), 4° C., for 10 hrs followed by 28 OC dissociation due to elimination of residual hydrate structure
Total 10 cycles hydrate formation and dissociation

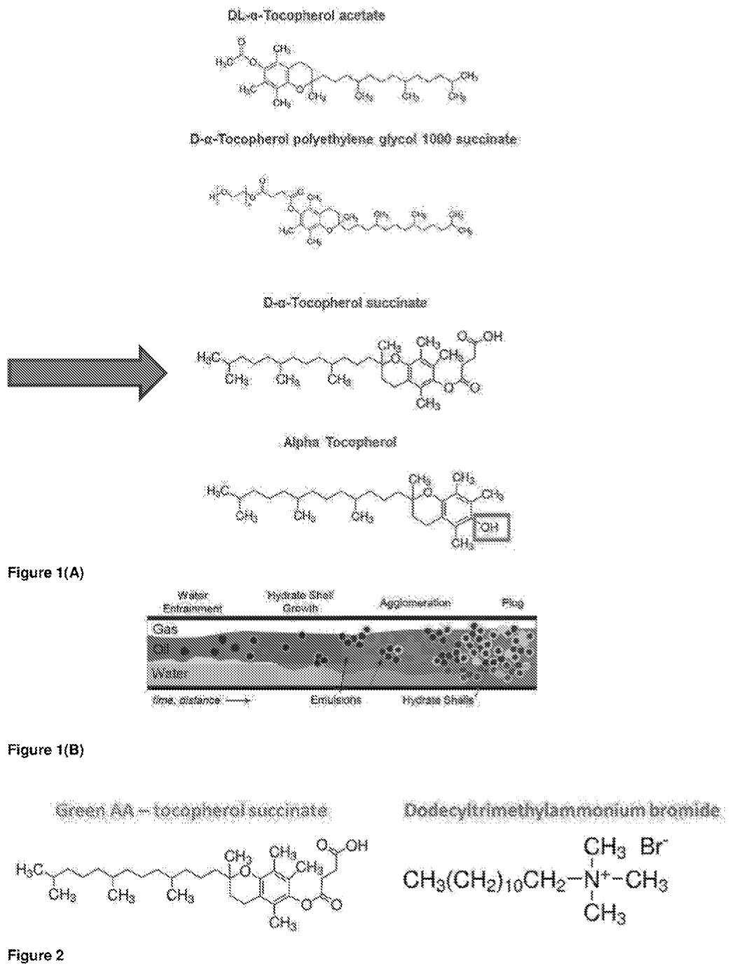

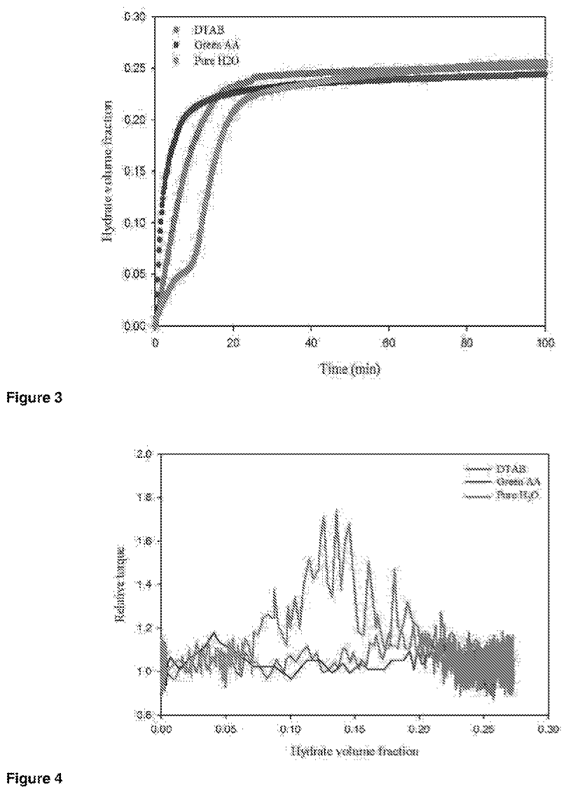

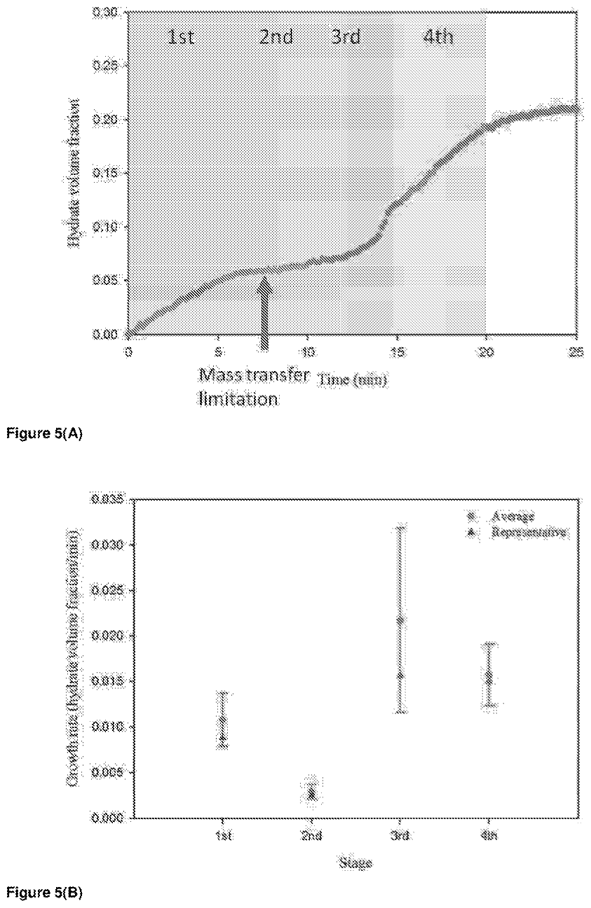

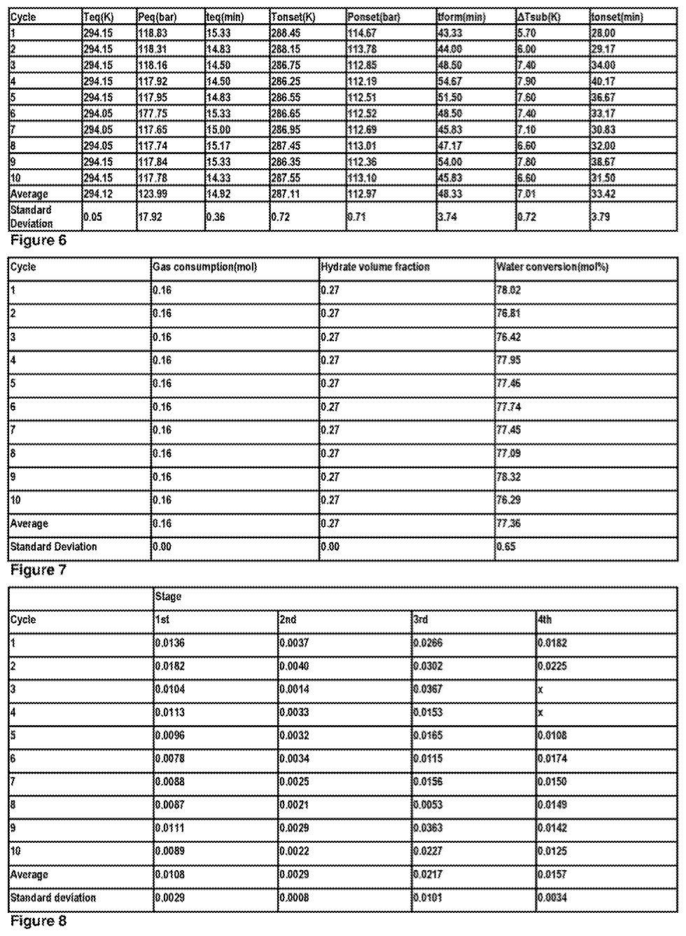

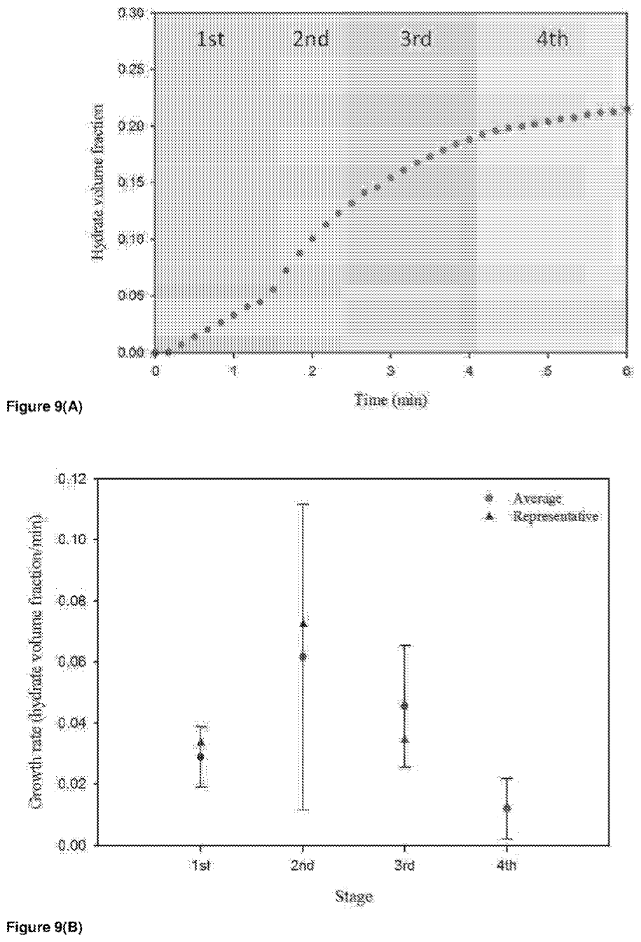

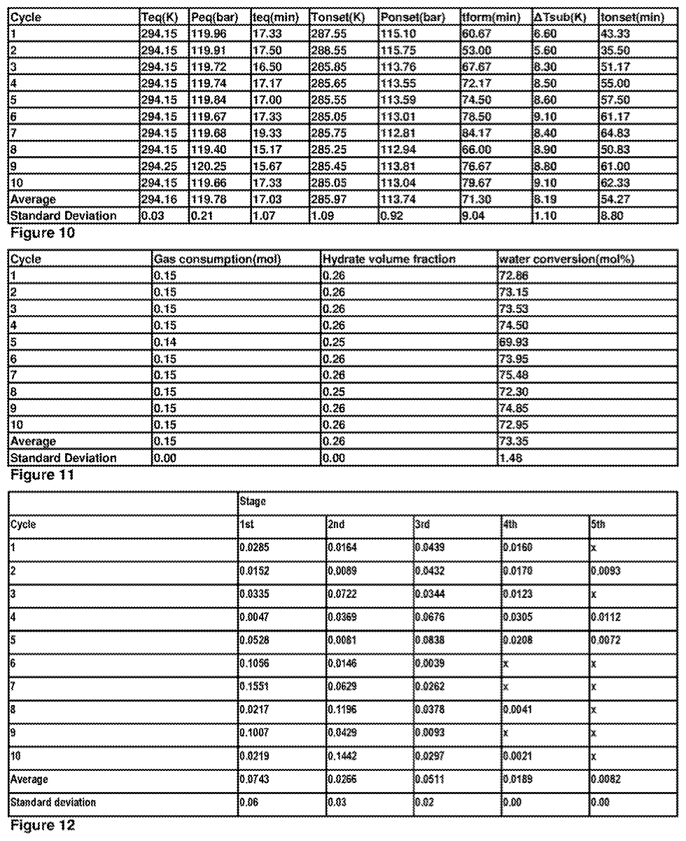

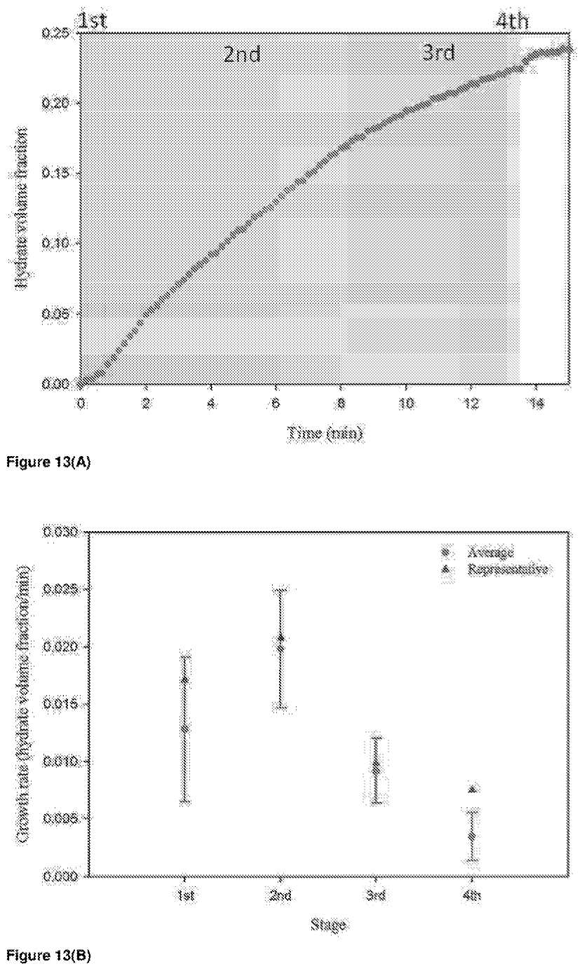

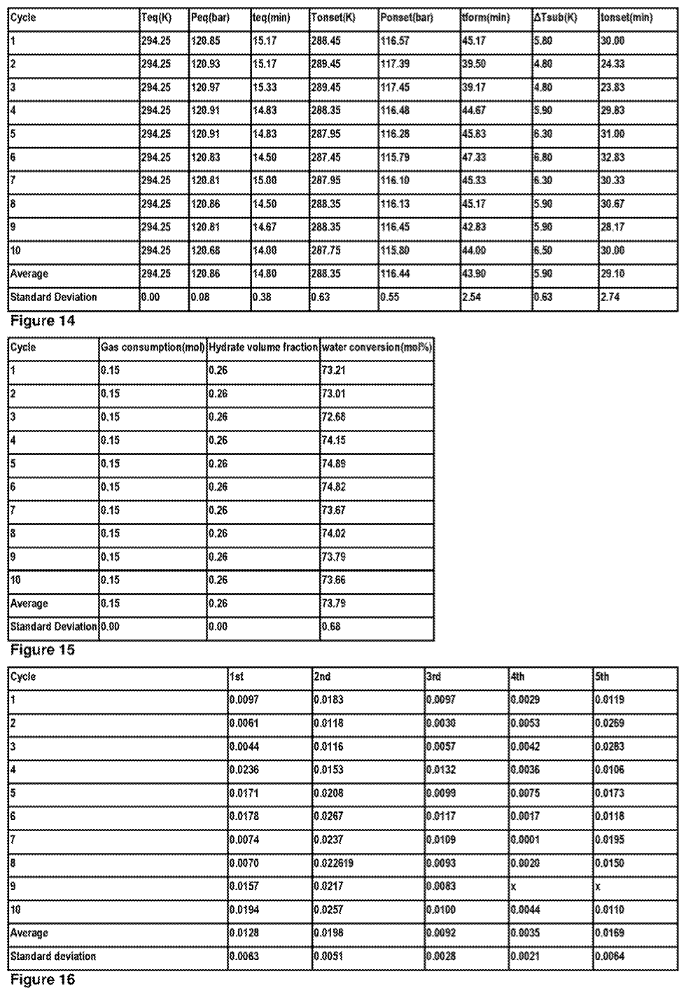

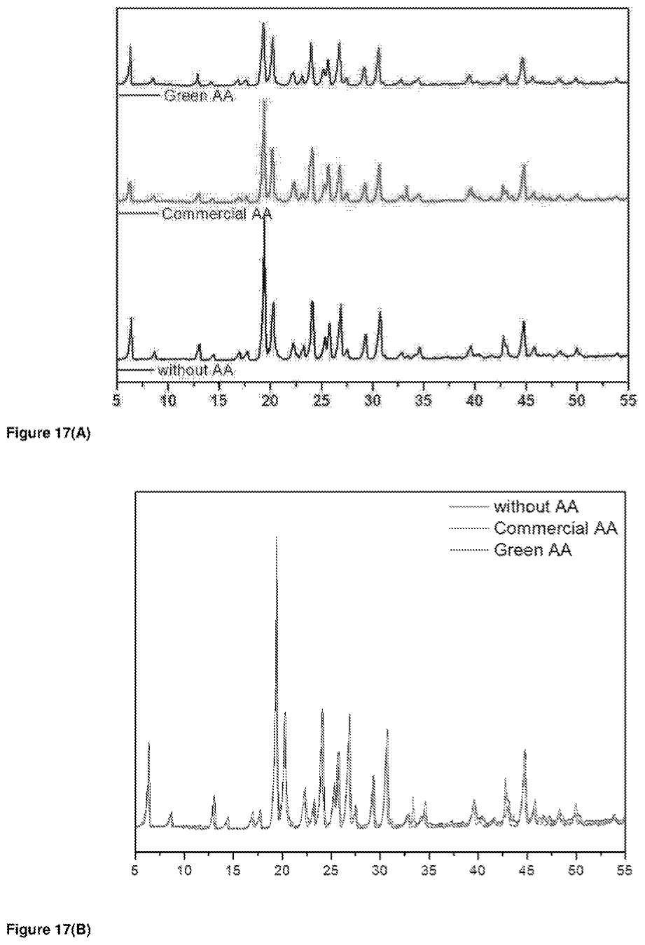

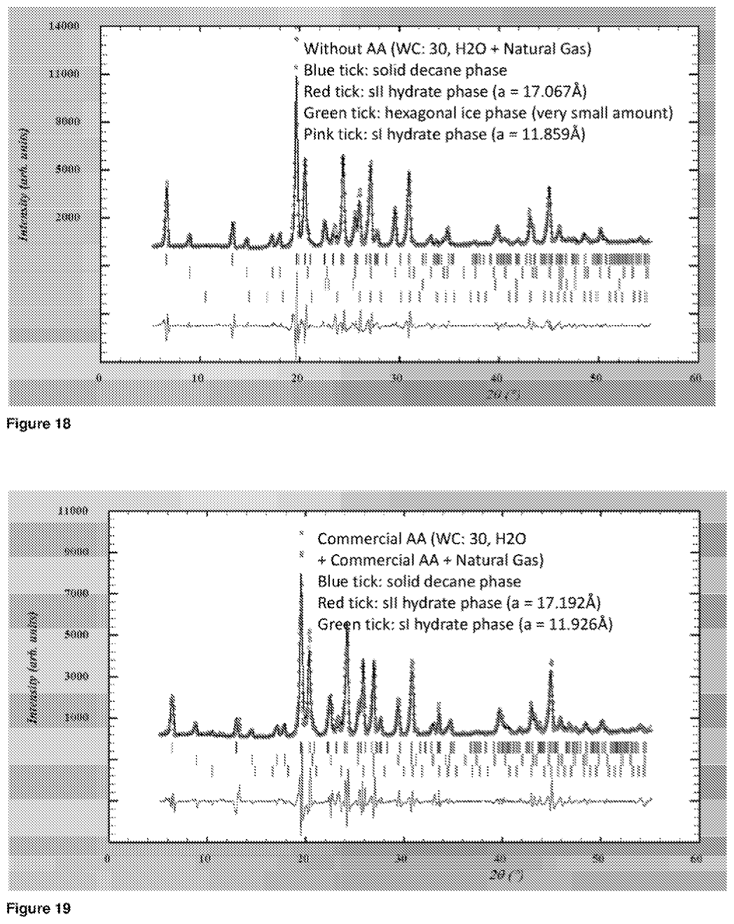

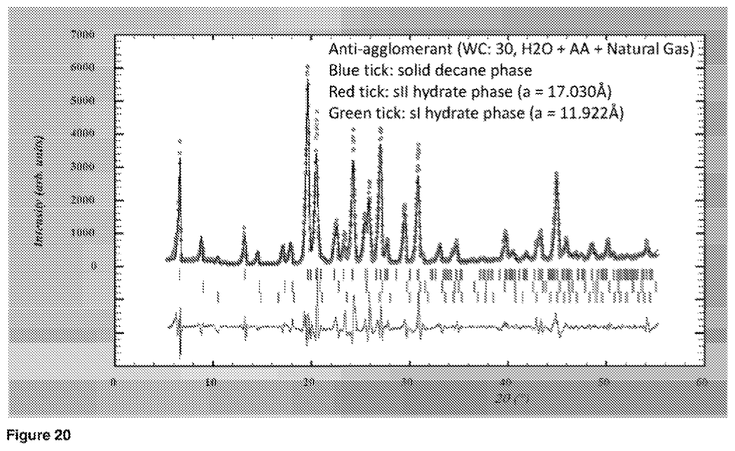

The initial testing involved use of commercially available tocopherol moieties as lipophilic portion of the anti-agglomerant molecules of the invention. The commercially available tocopherol products are shown in FIG. 1.

The experiments compare hydration formation and agglomeration in three systems comprising pure water (system 1), anti-agglomerant, tocopherol succinate (system 2), and commercial AA, dodecytrimethylammonium bromide “DTAB” (system 3).

The structures of the comparative AAs used are shown in FIG. 2.

### Experimental Method

For each system considered, a total liquid volume of 80 mL of 0.5 wt % anti-agglomerant (system 2) or 0.5 wt % of commercial DTAB AA solution (system 3) or pure water (system 1) was loaded into the autoclave cell which had an internal volume of 360 mL under a watercut 30 environment. The cell was immersed in a temperature-controlled liquid bath connected to an external refrigerated heater. A platinum resistance thermometer monitored the temperature of the liquid phase inside of the autoclave with an uncertainty of 0.15° C. The pressure was measured by a pressure transducer with an uncertainty of 0.1 bar in a range of 0-200 bar. Temperature and pressure were recorded using a data acquisition system.

The experiment was commenced by loading the 80 ml of liquid phase into the autoclave cell.

After purging the cell three times with the natural gas, the autoclave was pressurized to 120 bar at 24° C., while stirring at 600 rpm to saturate the liquid phase with gas. Once the pressure and temperature reached steady-state, the cell was cooled to 4° C., within 2 hours (0.25 Kmin⁻¹) and kept for 10 hours at the temperature. During this time, torque, pressure and temperature were continuously monitored. The dissociation of hydrate was carried out at 28° C., for a sufficient time to remove the residual hydrate structures prior to the next run.

Ten experiments were carried out for each system to determine averages for the hydrate formation including hydrate growth rate, hydrate formation kinetics, hydrate characterisation and dissociation onset time, and hydrate volume fraction.

### Results

The anti-agglomerant, tocopherol succinate, showed a degree of kinetic hydrate inhibition ability based on the increased hydrate formation onset time and subcooling of the temperature of the pure water system 1 (pure water: 7.01K, 33.42 min→anti-agglomerant: 8.19 K, 54.27 min).

In contrast, from the kinetics perspective, the commercial AA (DTAB) resulted in the slight promotion of hydrate formation in terms of time and temperature (pure water: 7.01K, 33.42 min→DTAB: 5.9 K, 29.10 min). Furthermore, the results indicate that systems 2 and 3 containing anti-agglomerant and conventional AA respectively, both overall resulted in a less conversion of water to hydrate than which occurred in the pure water system 1. In addition, both systems 2 and 3 comprising AA showed a similar conversion ratio (pure water: 77.36%, DTAB: 73.79%, and Anti-agglomerant: 73.35%).

During hydrate formation in the pure water system 1, the torque rapidly increased in the range of 0.5-0.15 hydrate volume fraction (τ_{rel,max}: 1.74). This observation is consistent with the rapid rate of growth rate in the second to third stage of the hydrate growth rate curve. As a result of this phenomenon, the resistance to flow in the system suddenly increases as indicated by the sudden torque increase observed.

For the DTAB system 3, the maximum torque (τ_{rel,max}: 1.32) was recorded between 0.15 and 0.25 hydrate volume fraction. Like DTAB, the Anti-agglomerant showed a similar trend (τ_{rel,max}: 1.18).

With regard to growth rate, the hydrate volume fraction was mainly observed in the second stage showing the highest average growth rate.

For the commercial AA (system 3) and the anti-agglomerant (system 2), the growth rate trend showed a similar pattern showing the highest value seen in the second stage followed by a decreased rate. Unlike the AA systems, the growth rate stagnated in the pure water system due to mass transfer limitations. Rapid growth rate in the next step is cause by for the reason. The rapid torque increase is evidenced by the rapid growth rate with concern of hydrate fraction.

Preferably, the anti-agglomerant is a hydratephillic type AA and is not emulsifying which is likely to explain why no emulsion forms. This is indicative of demonstrating that the hydrophilic head group of anti-agglomerant is interacting with the gas hydrate. This behaviour is in line with that of DTAB in system 3. In contrast to the anti-agglomerants described herein, the commercial AAs are charged making them sensitive to ions present and insoluble in the continuous phase.

As the anti-agglomerant is a vitamin-E based anti-agglomerant it is less toxic to the environment in comparison with quaternary ammonium AA such as DTAB.

The diffraction studies indicate that regardless of the nature of the AA used the XRD pattern of the solids formed are very similar indicating essentially the same hydrate mixtures are formed in each of systems 1-3. In short, there is a combination of structure I and structure II hydrate formed in all three systems considered. It is clear therefore that the presence of the anti-agglomerant does not alter the structure of the hydrate formed.

The corrosion inhibition of tocopherol carbon steel was tested in an aqueous corrosion fluid. The weight-loss measurement was conducted in open vials. The solutions used were 2M HCl with or without 500 ppm polymers (Blank). The carbon steel samples were cut into ø13×1 mm dimensions and were washed and degreased by ethanol after polish. They were then rinsed and dried with distilled water. These coupons with freshly prepared surface were then immersed in the HCl/polymers solutions for 120 h. At specific time intervals, the coupons were withdrawn from the solutions and weighted after being rinsed by ethanol and pure water. The immersion tests were totally repeated 5 times to acquire relatively accuracy data. The experiment compares the measure of weight loss during the exposure period. Where more corrosion occurs, a higher weight loss of the starting sample is observed. The data shows that (+) alpha-tocopherol (2.88% loss) and racemic alpha tocopherol (2.69% loss) has some corrosion inhibition ability compared to the blank sample (3.4% loss). As this method is aqueous-based, oil soluble inhibitors are expected to perform better.

### CLAUSES

1. A process for inhibiting gas hydrate agglomeration in a hydrocarbon phase, preferably natural gas, comprising the steps of:
(i) providing at least one oil soluble anti-agglomerant derived from a lipophilic, oil soluble naturally occurring product, a precursor to, a metabolite or a derivative thereof and comprising at least one non-ionic hydrophilic head group;
(ii) generating repulsive forces between gas hydrates in the hydrocarbon phase by contacting the gas hydrates with the at least one anti-agglomerant to form a plurality of dispersed gas hydrate-anti-agglomerant anti-agglomerant associated particles.
2. A process for inhibiting gas hydrate crystal agglomeration in a hydrocarbon phase, wherein under gas hydrate crystal formation conditions, one or more of the hydrocarbons are trapped within structures formed from water molecules present with the one or more hydrocarbons, said method comprising the steps of:
(i) providing at least one oil soluble anti-agglomerant derived from a lipophilic, oil soluble naturally occurring product, a precursor to, a metabolite or a derivative thereof and comprising at least one non-ionic hydrophilic head group;
(ii) contacting the gas hydrate crystals during or after their formation with the at least one anti-agglomerant whereby the hydrophilic head groups of the at least one anti-agglomerant associates with the water molecules of the gas hydrate crystals to form a plurality of associated gas crystal-anti-agglomerant particles whereby repulsive forces between the lipophilic tail groups of the associated gas crystal-anti-agglomerant particle formed prevent agglomeration of the gas hydrate crystals into gas hydrate clusters.
3. A process of inhibiting the formation of gas hydrate blockages in a hydrocarbon well, pipeline and/or conduit carrying a hydrocarbon phase, the process comprising the steps of:
preventing a gas hydrate of blockage size forming in the hydrocarbon phase by maintaining a dispersion of gas hydrate crystals formed in the hydrocarbon phase by contacting the gas hydrate crystals with at least one oil soluble anti-agglomerant derived from a lipophilic, oil soluble naturally occurring product, a precursor to, a metabolite or a derivative thereof and comprising at least one non-ionic hydrophilic head group thereby forming a plurality of associated gas crystal-anti-agglomerant particles,
whereby repulsive forces between the associated gas crystal-anti-agglomerant particles prevent agglomeration of the gas hydrate crystals into a solid blockage size plug.
4. A process according to any one of the preceding clauses, whereby the degree of lipophilicity and hydrophilicity of the respective lipophilic and hydrophilicity is sufficiently balanced to provide an oil soluble anti-agglomerant that is capable of associating with hydrates such that the resultant forces between the gas hydrate-anti-agglomerant associated particles maintain the gas hydrate-anti-agglomerant associated particles in substantially dispersed form in the hydrocarbon phase.
5. A process according to any one of the preceding clauses, wherein the repulsive forces between the gas hydrate-anti-agglomerant associated particles prevents agglomeration of the particles into larger gas hydrate clusters of a size that forms a solid hydrate pipeline or conduit blockage.
6. A process according to any one of the preceding clauses, wherein the hydrocarbon phase is a water-in-hydrocarbon dispersion.
7. A process according to any one of the preceding clauses, wherein the hydrocarbon phase is transported through a line, for example, a well, flowline, pipe or conduit.
8. A process for inhibiting gas hydrate agglomeration in a slurry in a transport line comprising a water-in-hydrocarbon phase having a plurality of water droplets dispersed in a continuous hydrocarbon phase, said method comprising the steps of:
(i) providing at least one oil soluble anti-agglomerant derived from a lipophilic, oil soluble naturally occurring product, a precursor to, a metabolite or a derivative thereof and comprising at least one non-ionic hydrophilic head group, to the water-in-hydrocarbon dispersion in the transport line;
(ii) allowing the anti-agglomerant to associate with gas hydrates formed at an interface of the water-hydrocarbon phase to form a slurry comprising a dispersion of gas hydrate-anti-agglomerant associated particles in the hydrocarbon continuous phase,
whereby repulsive forces between the gas hydrate-anti-agglomerant associated particles and/or attractive forces between the gas hydrate-anti-agglomerant associated particles and the hydrocarbon continuous phase of the slurry maintain the gas hydrate-anti-agglomerant associated particles in substantially dispersed form.
9. A process according to any one of the preceding clauses wherein the dispersion has a relative viscosity lower than that of slurry comprising equivalent particles formed under the same conditions in the absence of the oil soluble anti-agglomerant.
10. A process according to any one of the proceeding clauses, wherein the hydrocarbon phase has an associated watercut of from 1 to 100%, more preferably from 5 to 60%, and most preferably from 10 to 50%.
11. A process according to any one of the proceeding clauses, further comprising the step of monitoring the risk of plugging or blockage by the observation of one or more of: a decrease in flow rate, a pressure drop along a flowline, an increase in the hydrate volume fraction in the line, and an increase in the hydrate/hydrocarbon slurry relative viscosity, whereby observation of one or more of pressure drop, hydrate volume fraction and relative viscosity indicates that anti-agglomerant is required in the hydrocarbon phase, for example, wherein anti-agglomerant is added such that ΔP_{flowline} is maintained at a value <300 psi, Φ_{hyd} is maintained at a value <0.10, whereby Φ_{hyd} is the hydrate volume fraction in the line, and/or μᵣ is maintained at a value <10, whereby μᵣ is the hydrate slurry relative viscosity, a decrease in flow rate of a 20%.
12. A process according to any one of the proceeding clauses, wherein the preferred anti-agglomerant does not generate an oil-in-water emulsion.
13. A process according to any one of the proceeding clauses, wherein the non-ionic hydrophilic head group s derived from a natural source or is a naturally occurring product, a precursor to, a metabolite or a derivative thereof, preferably, the anti-agglomerant is environmentally benign, biodegradable, and/or non-toxic to plants, animals, marine life and/or insects.
14. A process according to any one of the proceeding clauses, wherein the anti-agglomerant is derived from a lipid that is derived from ketoacyl or isoprene precursors or building blocks, for example, a nonsaponifiable lipid, preferably selected from the group consisting of: a fat soluble vitamin, fat soluble terpene and a fat soluble steroid, fat soluble derivatives, fat soluble precursors, or fat soluble metabolite thereof.
15. A process according to any one of the proceeding clauses, wherein the anti-agglomerant comprises at least one lipophilic tail being a lipid that is derived from ketoacyl or isoprene precursors or building blocks, for example, a nonsaponifiable lipid, preferably selected from the group consisting of: a fat soluble vitamin, fat soluble terpene and a fat soluble steroid, fat soluble derivatives, fat soluble precursors, or fat soluble metabolite thereof.
16. A process according to any one of the proceeding clause, wherein the biodegradable naturally occurring product is an oil soluble substance selected from the group of naturally occurring lipids consisting of: Fatty Acyls [FA], Glycerolipids [GL], Sphingolipids [SP], Sterol Lipids [ST], Prenol Lipids [PR], Saccharolipids [SL], and Polyketides [PK].
17. A process according to any one of the proceeding clauses, wherein the anti-agglomerant comprises a fused ring system having from 2 to 10 fused 4, 5 or 6-membered rings, which are saturated or unsaturated rings, for example an optionally substituted chromane ring system or an optionally substituted gonane, optionally substituted sterol, optionally substituted sterone, optionally substituted phytosterol, optionally substituted sterane, cholestane, cholestene or other related ring system or secosteorid system, for example, comprising three fused 6-membered cyclohexane rings and one fused five membered ring, wherein the optional substituents are independently selected from one or more of C₁-C₆ alkyl, hydroxyl, acyl, carboxylate, halide, amine or amide functionalities.
18. A process according to any one of the proceeding clauses, the anti-agglomerant has the following general structure:

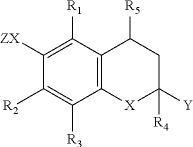

wherein
each X is independently —(CH₂)ₙ—, where n is 0 to 4, O, N or S;
when X outside the ring is —(CH₂)ₙ— and n is 1 to 4, Z is selected from the group of functionalities consisting of: optionally substituted alkyl carboxylate, optionally substituted alkyl dicarboxylate, optionally substituted alkyl dicarboxylate ester, optionally substituted alkyl amide, optionally substituted alkyl ether, optionally substituted alkyl ester;
when X outside the ring is O, Z is selected from the group of functionalities consisting of: optionally substituted carboxylate, optionally substituted dicarboxylate, optionally substituted dicarboxylate ester, optionally substituted amide, optionally substituted ether, optionally substituted ester;
when X outside the ring is N, Z is selected from the group of functionalities consisting of: optionally substituted amine, optionally substituted amide, optionally substituted azo, optionally substituted azoxy, optionally substituted carbamate, optionally substituted diazo, optionally substituted diazonium, optionally substituted guanidine, optionally substituted hydrazine, optionally substituted hydrazole, optionally substituted imine; optionally substituted carbonic acid bisamides, optionally substituted oximes, optionally substituted nitrones, and optionally substituted nitriles; when X outside the ring is S, Z is selected from the group of functionalities consisting of: thiol, selfenic acid, optionally substituted sulfide, optionally substituted disulfide, optionally substituted sulfinic acid, optionally substituted sulfinic acid ester, optionally substituted sulfonic acid, optionally substituted sulfonic acid ester;
Y is an optionally substituted branched or unbranched alkyl, an optionally substituted branched or unbranched alkenyl or an optionally substituted branched or unbranched alkynl;
R¹, R², R³, R⁴ and R⁵ are independently selected from H, OR^{x} wherein R^{x} is H or optionally substituted branched or unbranched alkyl, an optionally substituted branched or unbranched alkenyl or an optionally substituted branched or unbranched alkynl;
wherein the optional substituents are independently selected from one or more of C₁-C₆ alkyl, hydroxyl, acyl, carboxylate, halide, amine or amide functionalities.
19. A process according to clause 18, wherein X outside the ring is O, and Z is selected from the group of functionalities consisting of: optionally substituted carboxylate, optionally substituted dicarboxylate, optionally substituted dicarboxylate ester, optionally substituted amide, optionally substituted ether, optionally substituted ester.
20 A process according to clause 18 or clause 19, wherein Z is a carboxylate, alkyl carboxylate, ester or alkyl ester functionality.
21. A process according to any one of clauses 18 to 20, wherein the optionally substituted dicarboxylate functionality is of formula —C(O)(L)ₙC(O)OR⁶, wherein L is a linker group, n is 1 to 10, R⁶ is H or branched or unbranched alkyl.
22. A process according to any one of clause 18 to 21, wherein the optionally substituted dicarboxylate functionality is selected from oxalate, malonate, succinate, glutarate, adipate, or pimelate.
23. A process according to any one of clause 18 to 22, wherein Y is an optionally substituted branched or unbranched C₃-C₂₅ alkyl, an optionally substituted branched or unbranched C₃-C₂₅ alkenyl or an optionally substituted branched or unbranched C₃-C₂₅ alkynl, more preferably, an optionally substituted branched or unbranched C₁₂-C₁₇ alkyl, an optionally substituted branched or unbranched C₁₂-C₁₇ alkenyl or an optionally substituted branched or unbranched C₅-C₁₇ alkynl.
24. A process according to any one of the preceding clauses, wherein the anti-agglomerant has the following general structure:

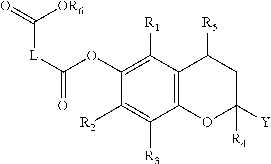

wherein
R¹, R², R³, R⁴ and R are independently H, OH, methyl, ethyl, propyl or butyl;
L is —(CH₂)ₙ-alkyl linker and n is 2, 3 or 4 and R⁶ is H;
Y is optionally substituted branched or unbranched C₁₂-C₁₇ alkyl, an optionally substituted branched or unbranched C₁₂-C₁₇ alkenyl or an optionally substituted branched or unbranched C₅-C₁₇ alkynl.
25. A process according to clause 24, wherein (i) R¹ is methyl, R² is methyl, R³ is methyl, R⁴ is methyl, and R⁵ is H; and Y is a saturated or unsaturated C₁₆ alkyl having a methyl branch at carbons 2, 8 and 12 of the C₁₆ alkyl chain, the anti-agglomerant is based on alpha-tocopherol or alpha-tocotrienol; (ii) R¹ is methyl, R² is H, R³ is methyl, R⁴ is methyl, and R⁵ is H; and Y is a saturated or unsaturated C₁₆ alkyl having a methyl branch at carbons 2, 8 and 12 of the C₁₆ alkyl chain, the anti-agglomerant is based on beta-tocopherol or beta-tocotrienol; (iii) R¹ is H, R² is H, R³ is methyl, R⁴ is methyl, and R⁵ is H; and Y is a saturated or unsaturated C₁₆ alkyl having a methyl branch at carbons 2, 8 and 12 of the C₁₆ alkyl chain, the anti-agglomerant is based on gamma-tocopherol or gamma-tocotrienol; (iv) R¹ is H, R² is H, R³ is methyl, R⁴ is methyl, and R⁵ is H; and Y is a saturated or unsaturated C₁₆ alkyl having a methyl branch at carbons 2, 8 and 12 of the C₁₆ alkyl chain, the anti-agglomerant is based on delta-tocopherol or delta-tocotrienol.
26. A process according to any one of the proceeding clauses, wherein the lipophilic tail group of the anti-agglomerant of the invention is based on a vitamin E based product such as alpha-tocopherol, alpha-tocotrienol, beta-tocotrienol, delta-tocotrienol, gamma-tocotrienol, didesmethyl tocotrienol, beta-tocopherol, alpha-tocopheronic acid, alpha-tocopheronolactone, gamma-tocopherol, and delta-tocopherol, most preferably, and alpha tocopherol succinate.
27. A process according to any one of clauses 1 to 17, wherein the anti-agglomerant has the following general formula:

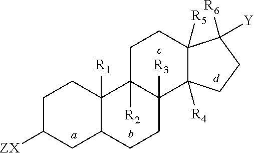

wherein
each of rings a, b, c, d can be independently saturated or unsaturated;
each X is independently —(CH₂)ₙ—, where n is 0 to 4, O, N or S;
when X is —(CH₂)ₙ— and n is 1 to 4, Z is selected from the group of functionalities consisting of: optionally substituted alkyl carboxylate, optionally substituted alkyl dicarboxylate, optionally substituted alkyl dicarboxylate ester, optionally substituted alkyl amide, optionally substituted alkyl ether, optionally substituted alkyl ester;
when X is O, Z is selected from the group of functionalities consisting of: optionally substituted carboxylate, optionally substituted dicarboxylate, optionally substituted dicarboxylate ester, optionally substituted amide, optionally substituted ether, optionally substituted ester;
when X is N, Z is selected from the group of functionalities consisting of: optionally substituted amine, optionally substituted amide, optionally substituted azo, optionally substituted azoxy, optionally substituted carbamate, optionally substituted diazo, optionally substituted diazonium, optionally substituted guanidine, optionally substituted hydrazine, optionally substituted hydrazole, optionally substituted imine; optionally substituted carbonic acid bisamides, optionally substituted oximes, optionally substituted nitrones, and optionally substituted nitriles; when X outside the ring is S, Z is selected from the group of functionalities consisting of: thiol, selfenic acid, optionally substituted sulfide, optionally substituted disulfide, optionally substituted sulfinic acid, optionally substituted sulfinic acid ester, optionally substituted sulfonic acid, optionally substituted sulfonic acid ester,
wherein the optional substituents are independently selected from one or more of C₁-C₆ alkyl, hydroxyl, acyl, carboxylate, halide, amine or amide functionalities;
Y is an optionally substituted branched or unbranched alkyl, an optionally substituted branched or unbranched alkenyl or an optionally substituted branched or unbranched alkynl;
R¹, R², R³, R⁴, R⁵ and R⁶ are independently selected from H, OH, COR^{x}, COOR^{x}, OR^{x} wherein R^{x} is H or optionally substituted branched or unbranched alkyl, an optionally substituted branched or unbranched alkenyl or an optionally substituted branched or unbranched alkynl, wherein the optional substituents are independently selected from one or more of C₁-C₆ alkyl, hydroxyl, acyl, carboxylate, halide, amine or amide functionalities.
28. A process according to clause 27, wherein the anti-agglomerant has general formula,

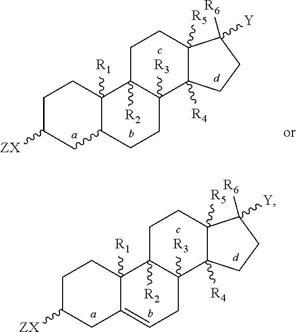

wherein
X is O;
Z is selected from the group of functionalities consisting of: optionally substituted carboxylate, optionally substituted dicarboxylate, optionally substituted dicarboxylate ester, optionally substituted amide, optionally substituted ether, optionally substituted ester;
R¹, R², R³, R⁴, R⁵ and R⁶ are independently selected from H, OR^{x} wherein R^{x} is H or optionally substituted branched or unbranched alkyl, an optionally substituted branched or unbranched alkenyl or an optionally substituted branched or unbranched alkynl;
wherein the optional substituents are independently selected from one or more of C₁-C₆ alkyl, hydroxyl, acyl, carboxylate, halide, amine or amide functionalities;
Y is an optionally substituted branched or unbranched alkyl, an optionally substituted branched or unbranched alkenyl or an optionally substituted branched or unbranched alkynl.
29. A process according to clauses 27 or 28, wherein Y is an optionally substituted branched or unbranched C₇-C₁₀ alkyl, an optionally substituted branched or unbranched C₇-C₁₀ alkenyl or an optionally substituted branched or unbranched C₇-C₁₀ alkynl.
30. A process according to any one of clauses 27 to 29, wherein the anti-agglomerant has the following general structure:

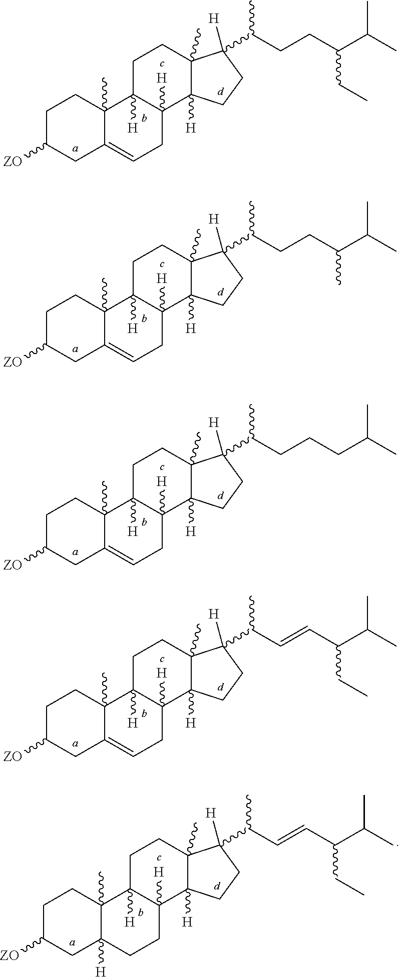

31. A process according to any one of clauses 27 to 30, the anti-agglomerant has the following general structure:

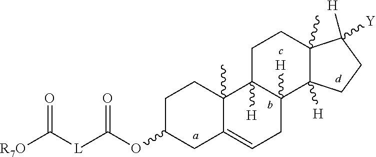

wherein
L is —(CH₂)ₙ-alkyl linker and n is 1, 2, 3 or 4, and R⁷ is H; and Y is an optionally substituted branched C₇-C₁₀ alkyl, an optionally substituted branched C₇-C₁₀ alkenyl.
32. A process according to any one of clauses 27 to 31, the hydrophilic head group is based on succinate, preferably, being contained within a sitosterol succinate anti-agglomerant molecule.
33. Use of an oil soluble anti-agglomerant to prevent gas hydrate agglomeration in a hydrocarbon phase, said at least one oil anti-agglomerant derived from a lipophilic, oil soluble naturally occurring product, a precursor to, a metabolite or a derivative thereof and comprising at least one non-ionic hydrophilic head group.
34. Use of an anti-agglomerant in the prevention of gas hydrate cluster formation, said anti-agglomerant derived from a lipophilic, oil soluble naturally occurring product, a precursor to, a metabolite or a derivative thereof and comprising at least one non-ionic hydrophilic head group.
35. Use according to clause 33 or 34 in oil and/or natural gas recovery, particularly where the hydrocarbon phase is transported in cold conditions, both onshore and offshore, preferably, as an alternative or supplement to thermodynamic inhibitors (MEG) in applications where thermodynamic inhibitors are used.
36. Use of an anti-agglomerant derived from a lipophilic, oil soluble naturally occurring product, a precursor to, a metabolite or a derivative thereof and comprising at least one non-ionic hydrophilic head group as a corrosion inhibitor.
37. Use according to clause 36, wherein the anti-agglomerant is used in the presence of a hydrocarbon phase.

1. A compound comprising at least one non-ionic hydrophilic head group component and at least one lipophilic tail component coupled by at least one linker moiety L, wherein L comprises:
(i) a group (chain)_{b}-R*, wherein R* is an amide (NHCO or CONH), an imine (C═N or N═C), an ester (C(O)O or OC(O)), ether or thioester (C(O)S or SC(O)) linkage between the at least one non-ionic hydrophilic head group component and the at least one lipophilic tail component, wherein b is 0 or 1, and (chain) is an optionally substituted alkyl, an optionally substituted alkenyl or an optionally substituted alkynyl functional group, or
(ii) ring system^{RS} which is a carbocyclic or heterocyclic ring system^{RS} linking the at least one non-ionic hydrophilic head group component and the at least one lipophilic tail component; and
wherein the at least one non-ionic hydrophilic head group component comprises an optionally substituted 5- or 6-membered carbocyclic or heterocyclic ring which is not a difatty ester substituted tetrahydrofuran ring, particularly not a distearate substituted fatty ester tetrahydrofuran ring, and
wherein at least one of the lipophilic tail components comprises at least one fatty chain, and
wherein the fatty chain is a branched or unbranched C₄-C₂₀ hydrocarbon chain, a fatty chain derived from a fatty acid, a fatty amine, a fatty ester, a fatty aldehyde, a fatty ether, a fatty nitrile, or a fatty alcohol.

2. A compound according to claim 1, wherein 5- or 6-membered carbocyclic or heterocyclic ring is not a fatty ester derivative of a tetrahydrofuran polyol ring, such that the compound is not an alkyl polyglucoside or a sorbitan fatty acid ester based compound such as sorbitan tristearate or sorbitan mono-oleoate.

3. A compound according to claim 1, having the following general structure: 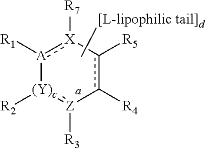
in which d is 1, 2 or 3, and
L represents
(i) (chain)_{b}-R*, wherein R* is an amide (NHCO or CONH), an imine (C═N or N═C), an ester (C(O)O or OC(O)), ether or thioester (C(O)S or SC(O)) linkage between ring^{a} which is an optionally substituted 5- or 6-membered carbocyclic or heterocyclic ring, and the lipophilic tail component at any one of R¹, R², R³, R⁴, R⁵, and R⁷ of ring, wherein b is 0 or 1, and (chain) is an optionally substituted alkyl, an optionally substituted alkenyl or an optionally substituted alkynyl functional group, or
(ii) a ring system^{RS} attached to ring^{a} as a substituent at any one of R¹, R¹, R³, R⁴, R⁵, and R⁷, and linking ring^{a} to the lipophilic tail, or a ring system^{RS} directly fused to ring^{a} at any adjacent pair of R¹ and R², R² and R³, R³ and R⁴, R⁴ and R⁵, R⁵ and R⁷ or R⁷ and R¹,
wherein the ring system^{RS} is an aromatic or aliphatic carbocyclic or heterocyclic ring system which may be saturated or unsaturated and which may be optionally substituted; and wherein
each of A, Y, Z of ring^{a} is independently CH, C, N, O, or S, and each instance of R¹, R², R³ is independently a lone pair or R⁶, provided that when A, Y and Z are independently O or S, the corresponding R¹, R² or R³ is a lone pair,
X is CH, C, or N, and
wherein when c is 0, Y is absent, or when c is 1, Y is present, provided that ring^{a} is not a difatty ester substituted tetrahydrofuran ring, particularly not a distearate substituted fatty ester tetrahydrofuran ring;
each instance of R⁴, R⁵, R⁶, and R⁷ when not L, is independently selected from hydrogen, halogen, hydroxyl, ether, alkyl hydroxyl, alkyl ether, oxo, thio, carboxylic acid, carboxylic acid ester, carboxylic acid alkyl ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfonic acid, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
when not L, each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted;
wherein the optional substituents are independently selected from with one or more of H, C₁-C₆ alkyl, acyl, carboxylic acid, carboxylate ester, alkyl carboxylic acid, alkyl carboxylate ester, hydroxyl, alkyl hydroxyl, sulfonic acid, amine, amide or halogen; and
wherein the at least one lipophilic tail component comprises at least one fatty chain.

4. A compound according to claim 3, wherein d is 1 or 2, preferably having one of the following general structures:

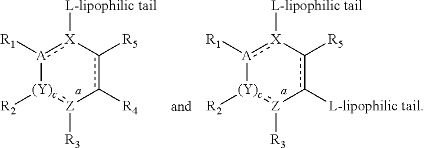

5. A compound according to claim 3, wherein the alkyl hydroxyl is (CH₂)ₙOH, where n is from 1 to 10, the carboxylic acid alkyl ester is C(O)O(CH₂)ₙH, n is from 1 to 10, and the alkyl carboxylic acid is (CH₂)ₙC(O)OH, wherein n is from 1 to 10.

6. A compound according to claim 1, the optionally substituted 5- or 6-membered carbocyclic or heterocyclic ring (ring^{a}) comprises a phenyl; a benzoic acid or alkyl esters thereof; an alkylbenzoic acid; an aromatic dicarboxylic acid; an aromatic dicarboxylic acid alkyl ester thereof; morpholinyl; tetrahydrofuranyl; oxopyrrolidnyl; pyrrolidnyl; piperidinyl; furanyl; imidazoyl; pyridinyl; pyrrolyl; benzodioxole; naphthyl; naphthoic acid; pyrrolylphenyl; or phenylsulfonic acid or esters thereof.

7. A compound according to claim 1, wherein R* is NHC(O), C(O)O, O, C(O)S, or N═CH.

8. A compound according to claim 1, comprising one of the following general formulas: 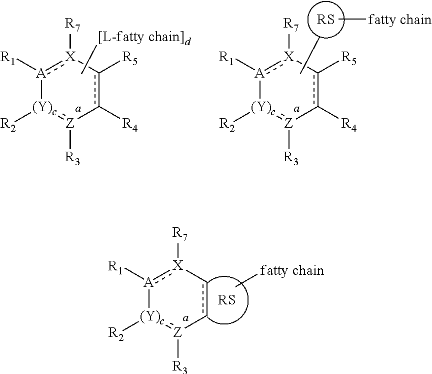
wherein the fused ring system^{RS} has 2 to 10 fused 4, 5 or 6-membered rings.

9. A compound according to claim 1, comprising a ring structure selected from the group consisting of:

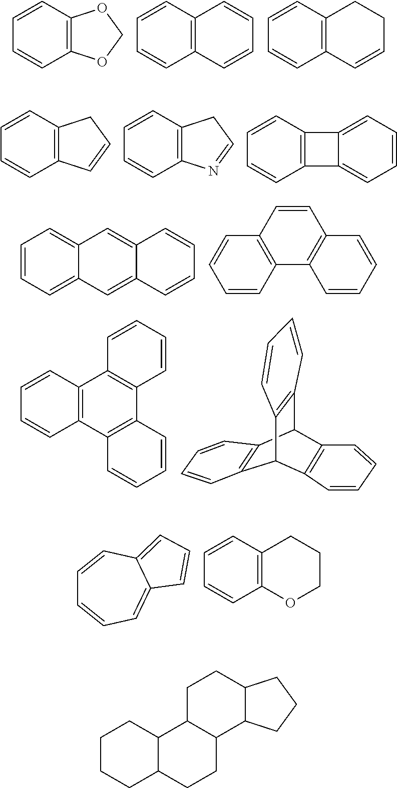

10. A compound according to claim 1, having the following general structure: 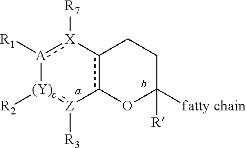
wherein when c is 0, Y is absent, or when c is 1, Y is present, and
each of A, Y, Z of ring^{a} is independently CH, C, N, O, or S, and each instance of R¹, R², R³ is independently a lone pair or R⁶, provided that when A, Y and Z are independently O or S, the corresponding R¹, R² or R³ is a lone pair,
X is CH, C, or N, and
each instance of R⁶, and R⁷ is independently selected from hydrogen, halogen, hydroxyl, ether, alkyl hydroxyl, alkyl ether, oxo, thio, carboxylic acid, carboxylic acid ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; and
wherein the optional substituents are independently selected from with one or more of C₁-C₆ alkyl, hydroxyl, amine, amide or halogen, and
wherein the fatty chain is a branched or unbranched C₄-C₂₀ hydrocarbon chain, a fatty chain derived from a fatty acid, a fatty amine, a fatty ester, a fatty aldehyde, a fatty ether, a fatty nitrile, or a fatty alcohol.

11. A compound according to claim 1 having the following general structure: 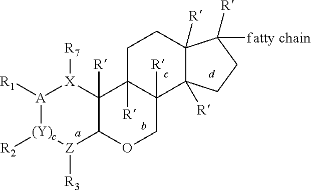
wherein ring^{a} and/or ring^{b} is saturated or unsaturated;
when c is 0, Y is absent, or when c is 1, Y is present, and
each of A, Y, Z of ring^{a} is independently C, C, N, O, or S, and each instance of R¹, R², R³ is independently a lone pair or R⁶, provided that when A, Y and Z are independently O or S, the corresponding R¹, R² or R³ is a lone pair,
X is CH, C, or N, and
each instance of R⁶, and R⁷ is independently selected from hydrogen, halogen, hydroxyl, ether, alkyl hydroxyl, alkyl ether, oxo, thio, carboxylic acid, carboxylic acid ester, alkyl carboxylic acid, alkyl carboxylic acid ester, amine, alkyl amine, sulfonic acid, alkyl sulfonic acid, alkyl, aldehyde, ketone, dicarboxyl including oxalate, malonate, succinate, glutarate, adipate, ester, alkyl ester, diester, dicarboxylate ester, guanidine, alkyl guanidine, imine, alkyl imine, imide, alkyl imide, sulfhydryl, sulfonyl, sulfinyl, sulfenyl, phosphoryl, diphosphoryl, thioester, alkyl thioester, a 5- or 6-membered carbocyclic or heterocyclic ring, any of which may be optionally substituted where structurally possible; or
each of R¹ and R², R² and R³, R³ and R⁴, and R⁴ and R⁵, when taken together independently form a 5- or 6-membered aromatic carbocyclic, aromatic heterocyclic ring, aliphatic carbocyclic or aliphatic heterocyclic ring which can be optionally substituted; and
wherein the optional substituents are independently selected from with one or more of C₁-C₆ alkyl, hydroxyl, amine, amide or halogen, and
wherein the fatty chain is a branched or unbranched C₄-C₂₀ hydrocarbon chain, a fatty chain derived from a fatty acid, a fatty amine, a fatty ester, a fatty aldehyde, a fatty ether, a fatty nitrile, or a fatty alcohol.

12. A compound according to claim 1, wherein the at least one at least one lipophilic tail component is a C₈, C₉, C₁₀, C₁₁, C₁₂, C₁₃ and C₁₆ fatty chains.

13. A compound according to claim 11, having the following general structure: 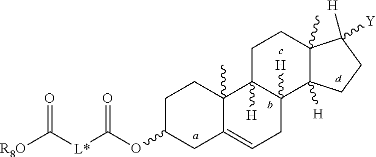
wherein
L* is (CH₂)ₙ alkyl linker and n is 1, 2, 3 or 4, and R⁸ is H; and
Y is an optionally substituted branched C₇-C₁₀ alkyl, or an optionally substituted branched C₇-C₁₀ alkenyl.

14. A compound selected from the group consisting of:

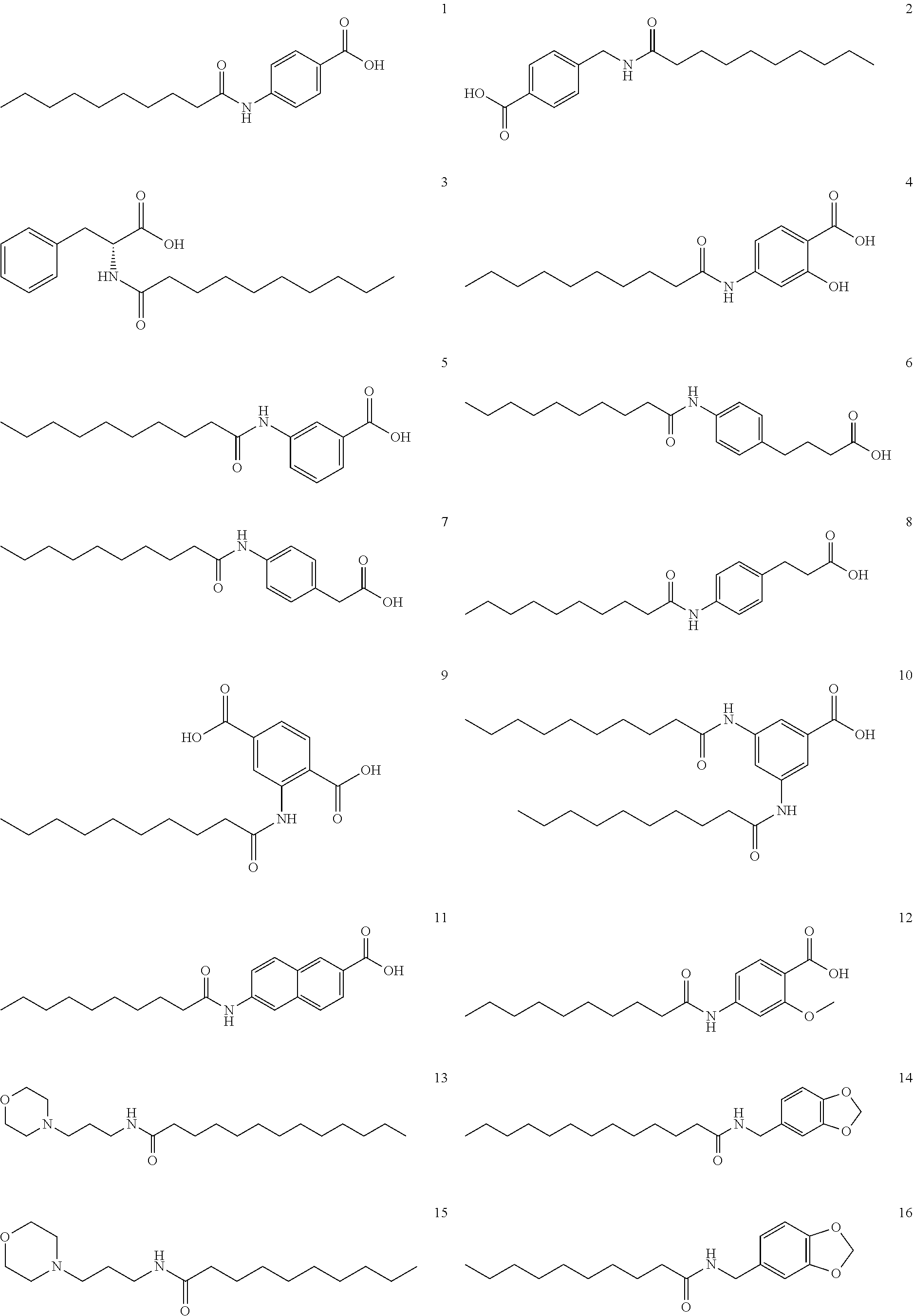

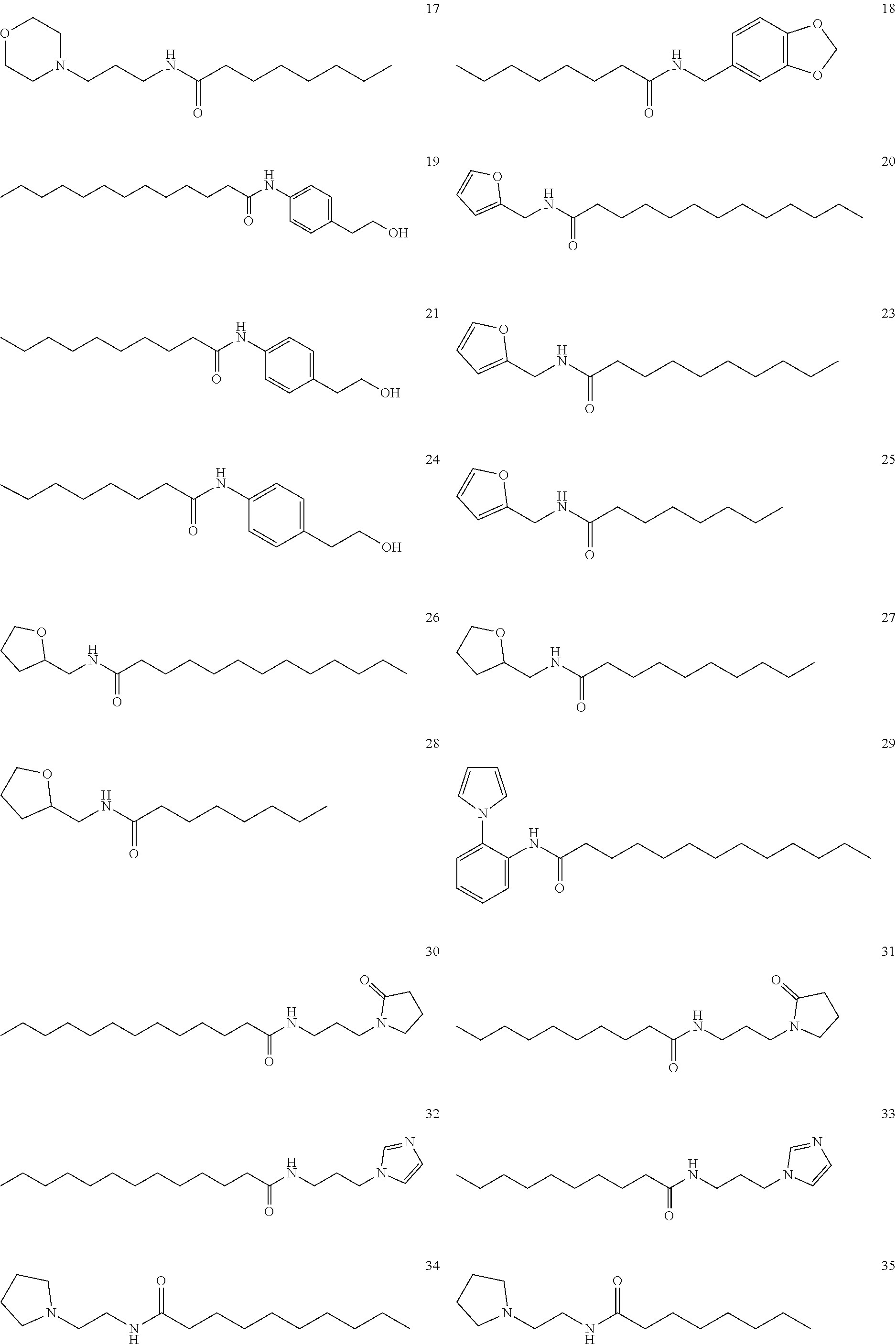

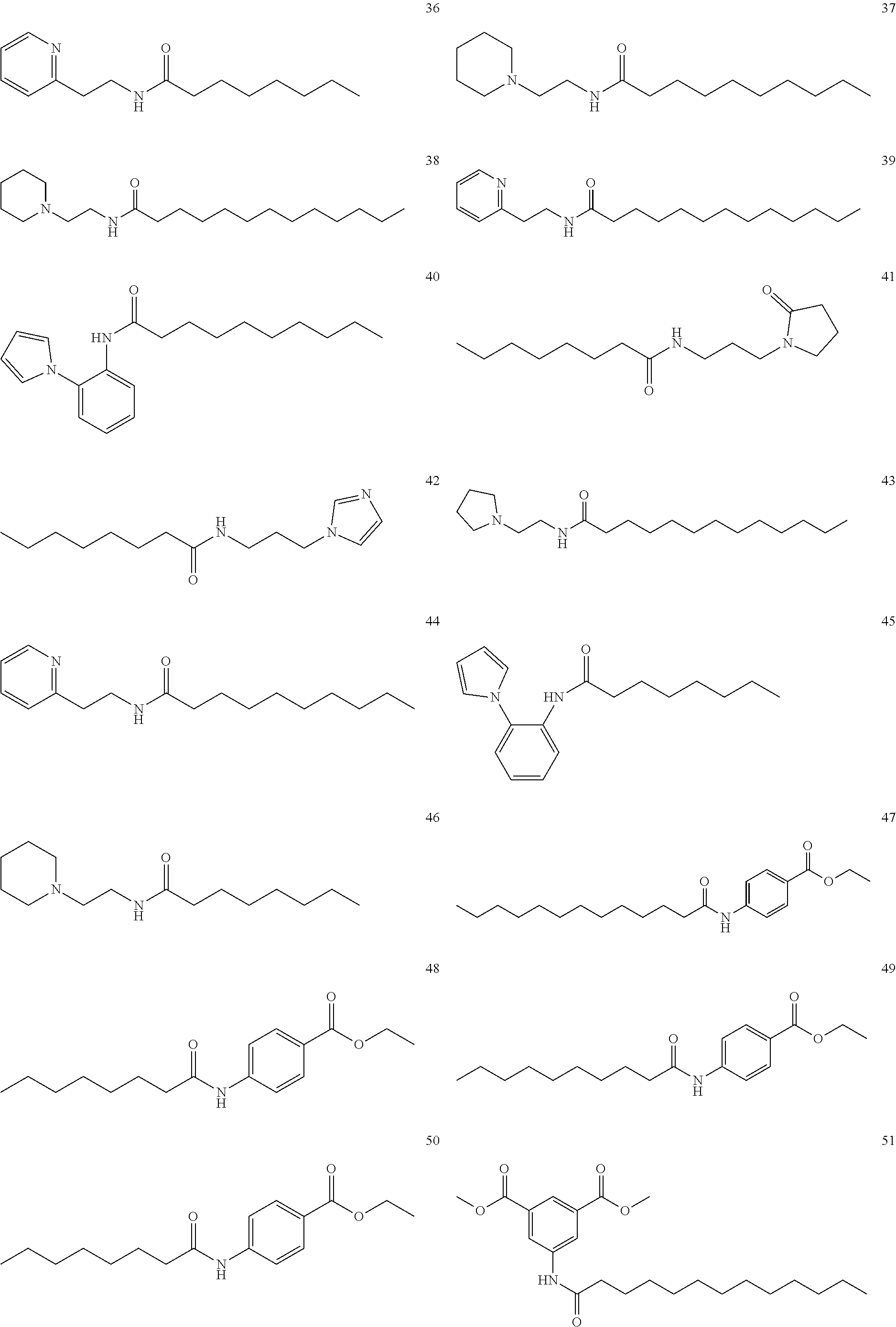

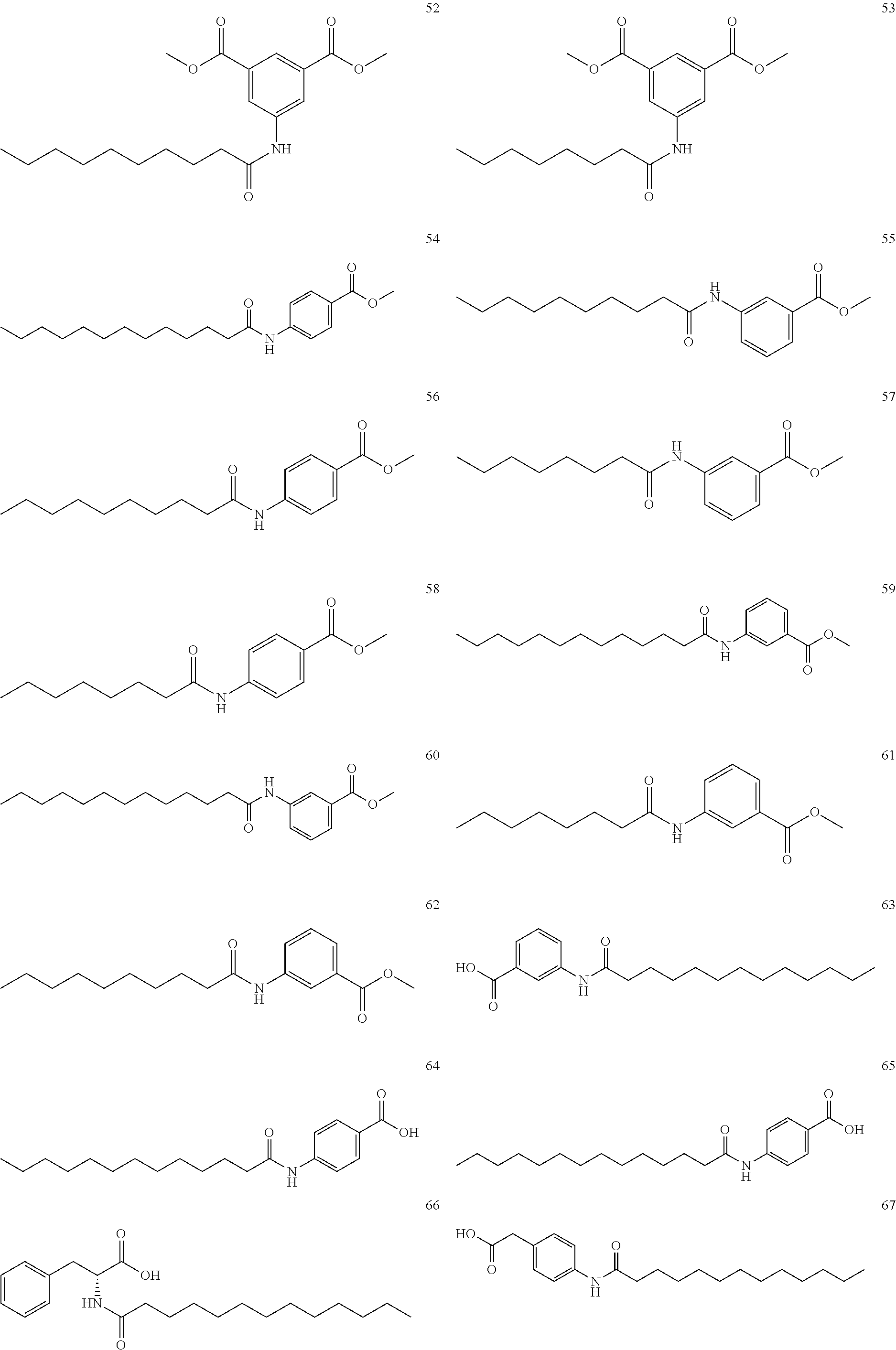

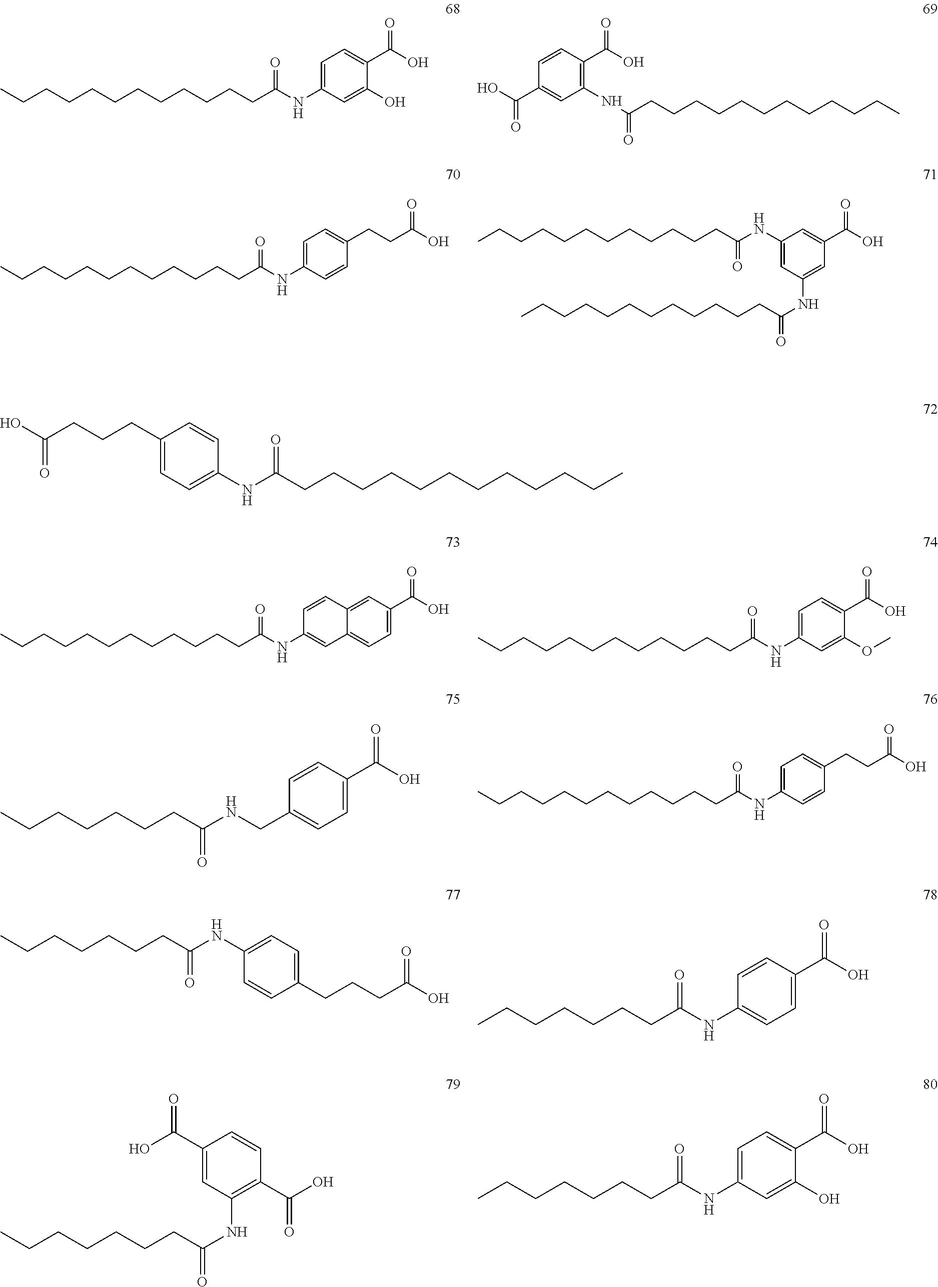

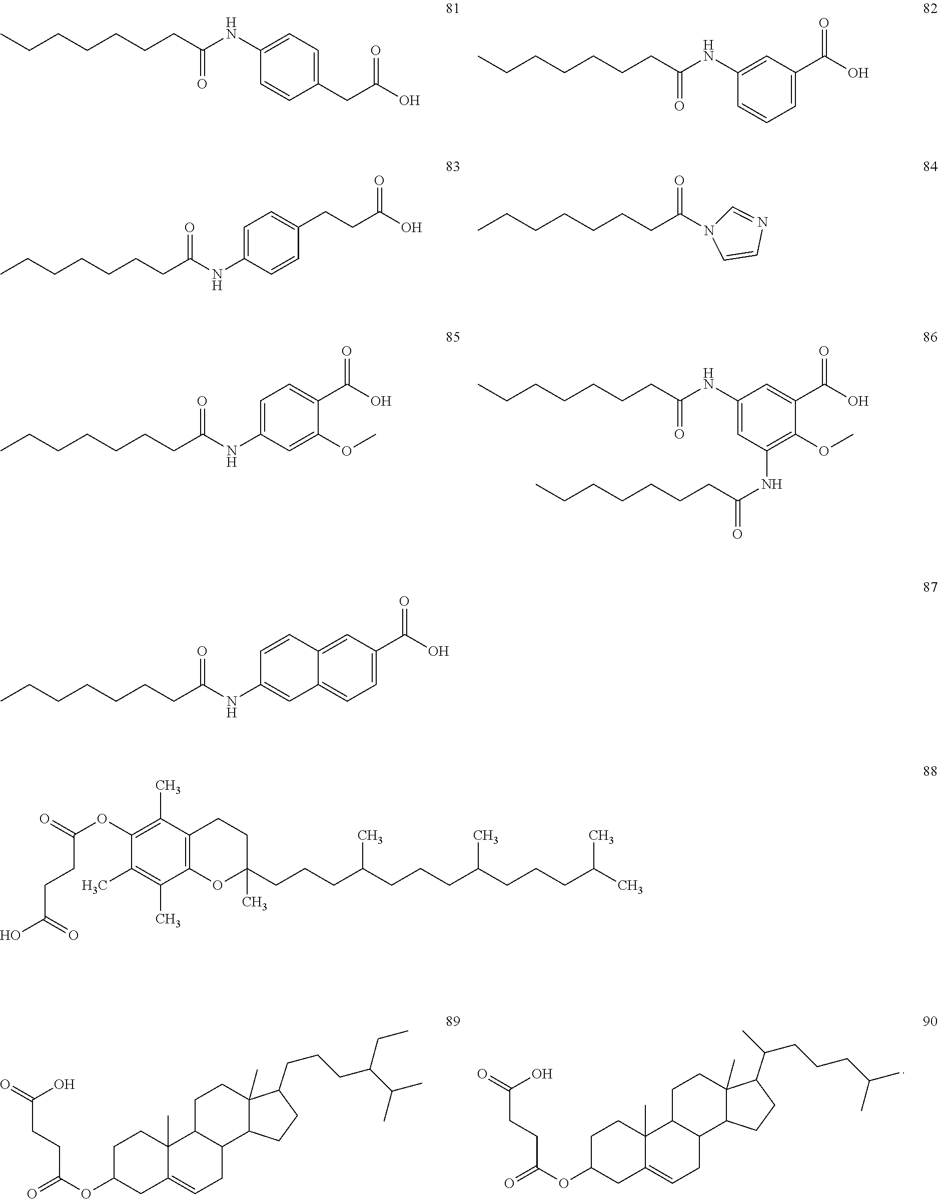

15. (canceled)

16. A compound according to claim 1, which is biodegradable meaning it produces a score of >20% biodegradability over 28 days as assessed by OECD 306: Biodegradability in Seawater, the Closed Bottle Test; has an ecotoxicity of LC50/EC50 of greater than 10 mg/L as determined by OECD 203: Fish, Acute Toxicity Test; and/or has a bio-concentration factor (BCF) larger than 100 or Log P_{ow}≤3 as determined by OECD 305: Bioaccumulation in Fish: Aqueous and Dietary Exposure.

17-28. (canceled)

29. A process for inhibiting gas hydrate agglomeration in a hydrocarbon phase comprising the steps of:
(i) providing at least one oil soluble anti-agglomerant compound as defined in claim 1;
(ii) generating repulsive forces between gas hydrates in the hydrocarbon phase by contacting the gas hydrates with the at least one anti-agglomerant to form a plurality of dispersed gas hydrate-anti-agglomerant anti-agglomerant associated particles.

30. A process according to claim 16 involving inhibiting the formation of gas hydrate blockages in a hydrocarbon well, pipeline and/or conduit carrying a hydrocarbon phase, the process comprising the steps of:
preventing a gas hydrate of blockage size forming in the hydrocarbon phase by maintaining a dispersion of gas hydrate crystals formed in the hydrocarbon phase by contacting the gas hydrate crystals with at least one oil soluble anti-agglomerant compound as defined in claim 1, thereby forming a plurality of associated gas crystal-anti-agglomerant particles,
whereby repulsive forces between the associated gas crystal-anti-agglomerant particles prevent agglomeration of the gas hydrate crystals into a solid blockage size plug.

31. A process according to claim 16 involving for inhibiting gas hydrate agglomeration in a slurry in a transport line comprising a water-in-hydrocarbon phase having a plurality of water droplets dispersed in a continuous hydrocarbon phase, said method comprising the steps of:
(i) providing at least one oil soluble anti-agglomerant compound as defined in claim 1, to the water-in-hydrocarbon dispersion in the transport line;
(ii) allowing the anti-agglomerant to associate with gas hydrates formed at an interface of the water-hydrocarbon phase to form a slurry comprising a dispersion of gas hydrate-anti-agglomerant associated particles in the hydrocarbon continuous phase,
whereby repulsive forces between the gas hydrate-anti-agglomerant associated particles and/or attractive forces between the gas hydrate-anti-agglomerant associated particles and the hydrocarbon continuous phase of the slurry maintain the gas hydrate-anti-agglomerant associated particles in substantially dispersed form.

32-36. (canceled)

32. A compound according to claim 6, the optionally substituted 5- or 6-membered carbocyclic or heterocyclic ring (ringa) comprises a benzoic acid ester; an alkylbenzoic acid; an aromatic dicarboxylic acid; or an aromatic dicarboxylic acid alkyl ester thereof; morpholinyl; tetrahydrofuranyl; oxopyrrolidnyl; pyrrolidnyl; piperidinyl; furanyl; imidazoyl; pyridinyl; pyrrolyl; benzodioxole; naphthyl; naphthoic acid; pyrrolylphenyl; or phenylsulfonic acid or esters thereof.

33. A compound according to claim 32, the optionally substituted 5- or 6-membered carbocyclic or heterocyclic ring (ringa) comprises methylbenzoate, ethyl benzoate, phenylacetic acid, phenylethanoic acid, phenylpropanoic acid, phenylbutanoic acid, phenylmethanoate, phenylethanoate, phenylpropanoate, phenylbutanoate; phthalic acid, terephthalic acid, isophthalic acid; methylphthalate, dimethylterephthalate, or dimethylisophthalate; morpholinyl; tetrahydrofuranyl; oxopyrrolidnyl; pyrrolidnyl; piperidinyl; furanyl; imidazoyl; pyridinyl; pyrrolyl; benzodioxole; naphthyl; naphthoic acid; pyrrolylphenyl; or phenylsulfonic acid or esters thereof.
